SEQUENCE LISTING (8 sequences)

SEQ ID NO: 1 - protein, 142 aa, Artificial Sequence
  Hemoglobin subunit alpha
MVLSPADKTNVKAAWGKVGAHAGEYGAEALERMFLSFPTTKTYFPHFDLSHGSAQVKGHGKKVADALTNAVAHVDDMPNALSALSDLHAHKLRVDPVNFKLLSHCLLVTLAAHLPAEFTPAVHASLDKFLASVSTVLTSKYR

SEQ ID NO: 2 - DNA, 575 bp, Artificial Sequence
  Hemoglobin subunit alpha
actcttctggtccccacagactcagagagaacccaccatggtgctgtctcctgccgacaagaccaacgtcaaggccgcctggggcaaggttggcgcgcacgctggcgagtatggtgcggaggccctggagaggatgttcctgtccttccccaccaccaagacctacttcccgcacttcgacctgagccacggctctgcccaggttaagggccacggcaagaaggtggccgacgcgctgaccaacgccgtggcgcacgtggacgacatgcccaacgcgctgtccgccctgagcgacctgcacgcgcacaagcttcgggtggacccggtcaacttcaagctcctaagccactgcctgctggtgaccctggccgcccacctccccgccgagttcacccctgcggtgcacgcctccctggacaagttcctggcttctgtgagcaccgtgctgacctccaaataccgttaagctggagcctcggtagcagttcctcctgccagatgggcctcccaacgggccctcctcccctccttgcaccggcccttcctggtctttgaataaagtctgagtgggcggc

SEQ ID NO: 3 - protein, 147 aa, Artificial Sequence
  Hemoglobin subunit beta
MVHLTPEEKSAVTALWGKVNVDEVGGEALGRLLVVYPWTQRFFESFGDLSTPDAVMGNPKVKAHGKKVLGAFSDGLAHLDNLKGTFATLSELHCDKLHVDPENFRLLGNVLVCVLAHHFGKEFTPPVQAAYQKVVAGVANALAHKYH

SEQ ID NO: 4 - DNA, 628 bp, Artificial Sequence
  Hemoglobin subunit beta
acatttgcttctgacacaactgtgttcactagcaacctcaaacagacaccatggtgcatctgactcctgaggagaagtctgccgttactgccctgtggggcaaggtgaacgtggatgaagttggtggtgaggccctgggcaggctgctggtggtctacccttggacccagaggttctttgagtcctttggggatctgtccactcctgatgctgttatgggcaaccctaaggtgaaggctcatggcaagaaagtgctcggtgcctttagtgatggcctggctcacctggacaacctcaagggcacctttgccacactgagtgagctgcactgtgacaagctgcacgtggatcctgagaacttcaggctcctgggcaacgtgctggtctgtgtgctggcccatcactttggcaaagaattcaccccaccagtgcaggctgcctatcagaaagtggtggctggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgcaa

SEQ ID NO: 5 - protein, 147 aa, Artificial Sequence
  Hemoglobin subunit gamma
MGHFTEEDKATITSLWGKVNVEDAGGETLGRLLVVYPWTQRFFDSFGNLSSASAIMGNPKVKAHGKKVLTSLGDAIKHLDDLKGTFAQLSELHCDKLHVDPENFKLLGNVLVTVLAIHFGKEFTPEVQASWQKMVTGVASALSSRYH

SEQ ID NO: 6 - DNA, 586 bp, Artificial Sequence
  Hemoglobin subunit gamma
acactcgcttctggaacgtctgaggttatcaataagctcctagtccagacgccatgggtcatttcacagaggaggacaaggctactatcacaagcctgtggggcaaggtgaatgtggaagatgctggaggagaaaccctgggaaggctcctggttgtctacccatggacccagaggttctttgacagctttggcaacctgtcctctgcctctgccatcatgggcaaccccaaagtcaaggcacatggcaagaaggtgctgacttccttgggagatgccataaagcacctggatgatctcaagggcacctttgcccagctgagtgaactgcactgtgacaagctgcatgtggatcctgagaacttcaagctcctgggaaatgtgctggtgaccgttttggcaatccatttcggcaaagaattcacccctgaggtgcaggcttcctggcagaagatggtgactggagtggccagtgccctgtcctccagataccactgagctcactgcccatgatgcagagctttcaaggataggctttattctgcaagcaatcaaataataaatctattctgctaagagatcacaca

SEQ ID NO: 7 - protein, 147 aa, Artificial Sequence
  Hemoglobin subunit gamma
MGHFTEEDKATITSLWGKVNVEDAGGETLGRLLVVYPWTQRFFDSFGNLSSASAIMGNPKVKAHGKKVLTSLGDATKHLDDLKGTFAQLSELHCDKLHVDPENFKLLGNVLVTVLAIHFGKEFTPEVQASWQKMVTAVASALSSRYH

SEQ ID NO: 8 - DNA, 584 bp, Artificial Sequence
  Hemoglobin subunit gamma
acactcgcttctggaacgtctgaggttatcaataagctcctagtccagacgccatgggtcatttcacagaggaggacaaggctactatcacaagcctgtggggcaaggtgaatgtggaagatgctggaggagaaaccctgggaaggctcctggttgtctacccatggacccagaggttctttgacagctttggcaacctgtcctctgcctctgccatcatgggcaaccccaaagtcaaggcacatggcaagaaggtgctgacttccttgggagatgccacaaagcacctggatgatctcaagggcacctttgcccagctgagtgaactgcactgtgacaagctgcatgtggatcctgagaacttcaagctcctgggaaatgtgctggtgaccgttttggcaatccatttcggcaaagaattcacccctgaggtgcaggcttcctggcagaagatggtgactgcagtggccagtgccctgtcctccagataccactgagctcactgcccatgattcagagctttcaaggataggctttattctgcaagcaatacaaataataaatctattctgctgagagatcac

(19) United States
(12) Patent Application Publication
(10) Pub. No.: US 2021/0324046 A1
(43) Pub. Date: Oct. 21, 2021

(54) SYSTEMS AND METHODS FOR MANUFACTURE OF ENDOTOXIN-FREE HEMOGLOBIN-BASED DRUG SUBSTANCE

(71) Applicant: Medical Technology Associates II, Inc., Malvern, PA (US)

(72) Inventor: Carl W. RAUSCH, Durham, NH (US)

(21) Appl. No.: 17/150,210

(22) Filed: Jan. 15, 2021

Related U.S. Application Data
(60) Provisional application No. 62/962,561, filed on Jan. 17, 2020.

(51) Int. Cl. C07K 14/805; C07K 1/34; C07K 1/14; C07K 1/36; C07K 1/16

(52) U.S. Cl. CPC C07K 14/805; C07K 1/34; C07K 1/16; C07K 1/36; C07K 1/145

(57) ABSTRACT

The present disclosure relates to methods and systems for manufacturing stabilized hemoglobin solutions. The methods and systems incorporate single use components for endotoxin-free formulation. The hemoglobin solutions may be substantially endotoxin-free and/or highly deoxygenated.

### CROSS-REFERENCE

This application claims the benefit of U.S. Provisional Application No. 62/962,561, filed Jan. 17, 2020, which application is incorporated herein by reference in its entirety.

### INCORPORATION OF THE SEQUENCE LISTING

The contents of the text file submitted electronically herewith are incorporated herein by reference in their entirety: a computer readable format copy of the Sequence Listing (filename: MTAI_003_02US_SeqList_ST25.TXT, date recorded: Jan. 14, 2021, file size: ˜9,460 bytes).

### FIELD OF THE DISCLOSURE

The present disclosure relates to methods and systems for the manufacture of stabilized hemoglobin solutions. The methods and systems may be employed to produce stabilized hemoglobin solutions that are substantially free of endotoxins, highly deoxygenated, highly concentrated, and/or suitable for human therapeutic use.

### BACKGROUND

In human beings and mammals, hemoglobin is the iron-containing oxygen-transport metalloprotein in red blood cells that carries oxygen from the lungs to the rest of the body (i.e., the tissues). There, it releases oxygen to permit aerobic respiration to provide energy to power the functions of the organism in the process of metabolism. A healthy individual has 12 to 20 grams of hemoglobin in every 100 mL of blood. Hemoglobin has an oxygen-binding capacity of 1.34 mL 02 per gram, which increases the total blood oxygen capacity seventy-fold compared to dissolved oxygen in blood. The mammalian hemoglobin molecule can bind up to four oxygen molecules. In most vertebrates, the hemoglobin molecule is an assembly of four globular protein subunits. Each subunit is composed of a protein chain tightly associated with a non-protein prosthetic heme group. Each protein chain arranges into a set of alpha-helix structural segments connected together in a globin fold arrangement. This folding pattern contains a pocket that strongly binds the heme group.

In the treatment of trauma patients, transfusion with whole allogeneic blood is ubiquitous. However, on the worldwide scale, there is a shortage of safe and viable allogeneic donor blood, a problem that is only projected to increase over time. In addition, whole blood transfusion comes with risks, including blood-borne diseases, fatal ABO-incompatibility, systemic inflammatory response, and multiple organ failure. In addition, whole human blood has a limited shelf life of 42 days and the available quantities are insufficient for emergency situations involving numerous traumatic injuries, such as in warfare or after a natural disaster.

Existing hemoglobin-based drugs and oxygen carriers include perfluorochemicals, synthesized hemoglobin analogues, liposome-encapsulated hemoglobin, chemically-modified hemoglobin, and hemoglobin-based oxygen carriers in which the hemoglobin molecules are crosslinked. The preparation of hemoglobin-based drugs includes several purification steps to remove agents and cellular components that cause severe immune responses. Unfortunately, existing methods of producing hemoglobin solutions derived from bovine blood utilize drug purification methodologies that do not completely remove contaminants, such as cell lipid layers and lipopolysaccharides (endotoxins) which can complex with the hemoglobin protein at any stage of handling given exposure to bacterial endotoxin materials. As such, there is a pressing need to provide methods of hemoglobin-based drug purification and handling that are more cost effective, have increased product purity, and produce better batch to batch reproducibility.

There is an unmet need for methods and systems to produce safe hemoglobin-based blood substitutes for human treatment.

### BRIEF SUMMARY

The present disclosure provides a method for manufacturing a stabilized hemoglobin composition, comprising: diluting a purified hemoglobin solution to a hemoglobin concentration of less than 30 g/L to produce a dilute hemoglobin solution; deoxygenating the dilute hemoglobin solution, thereby producing a deoxygenated hemoglobin solution; and polymerizing the deoxygenated hemoglobin solution, thereby producing a stabilized hemoglobin composition.

In some embodiments, the stabilized hemoglobin composition is substantially endotoxin-free. In some embodiments, the stabilized hemoglobin composition comprises fewer than 0.05 endotoxin units (EU) per milliliter (mL) (EU/mL). In some embodiments, said stabilized hemoglobin comprises less than 0.01, 0.05, 0.04, 0.03, 0.02, or 0.01 mg/mL of dissolved oxygen.

In some embodiments, the hemoglobin solution is derived from a crude hemoglobin solution obtained from red blood cells. In some embodiments, the red blood cells are isolated or derived from a non-human animal. In some embodiments, the non-human animal is a bovine. In some embodiments, the red blood cells are collected using a sterile container. In some embodiments, the sterile container is a single-use bag. In some embodiments, the sterile container contains an anticoagulant. In some embodiments, the anticoagulant is a citrate phosphate dextrose (CPD) anticoagulant. In some embodiments, the red blood cells are washed. In some embodiments, washing the red blood cells comprises straining, filtering, and/or washing the red blood cells with buffer solution. In some embodiments, the red blood cells are lysed, thereby producing the crude hemoglobin solution. In some embodiments, the lysing of the red blood cells is by a rapid decrease in osmotic pressure resulting in cell lysis. In some embodiments, the crude hemoglobin solution is purified by diafiltration, ultrafiltration, clarification, and/or chromatography, thereby producing the purified hemoglobin solution.

In some embodiments, the deoxygenation step comprises diafiltration against a degassing membrane with nitrogen flowing across the opposite side of the membrane. In some embodiments, the diafiltration against the degassing membrane continues until the dissolved oxygen level is below 0.1 mg/mL. In some embodiments, the diafiltration against the degassing membrane continues until the dissolved oxygen level is below 0.02 mg/mL. In some embodiments, the deoxygenated hemoglobin solution is concentrated prior to polymerization. In some embodiments, the deoxygenated hemoglobin solution is further filtered prior to polymerization. In some embodiments, the deoxygenated hemoglobin solution is polymerized by cross-linking with glutaraldehyde. In some embodiments, the method further comprises stopping the polymerizing step by adding sodium borohydride. In some embodiments, the deoxygenated hemoglobin solution is diafiltered and/or concentrated during the polymerizing step. In some embodiments, the stabilized hemoglobin composition is diafiltered and/or concentrated after sodium borohydride is added. In some embodiments, the stabilized hemoglobin composition is concentrated to a concentration of 50-100 g/L. In some embodiments, the stabilized hemoglobin composition is concentrated to a concentration of 100-150 g/L. In some embodiments, the stabilized hemoglobin composition is concentrated to a concentration of 150-200 g/L. In some embodiments, the stabilized hemoglobin composition comprises hemoglobin isolated or derived from a non-human animal. In some embodiments, the non-human animal is a bovine. In some embodiments, the stabilized hemoglobin composition is stable at an ambient temperature. In some embodiments, the stabilized hemoglobin composition is stable above a temperature of at least 4° C. In some embodiments, endotoxins comprise one or more of a cellular lipid, a cellular lipid layer and a lipopolysaccharide. In some embodiments, the one or more of a cellular lipid, a cellular lipid layer and a lipopolysaccharide is from a human cell. In some embodiments, the one or more of a cellular lipid, a cellular lipid layer and a lipopolysaccharide is from a non-human vertebrate cell. In some embodiments, the one or more of a cellular lipid, a cellular lipid layer and a lipopolysaccharide is isolated from a microbe. In some embodiments, the one or more of a cellular lipid, a cellular lipid layer and a lipopolysaccharide is isolated from a bacterium.

In some embodiments, the stabilized hemoglobin composition has an average molecular weight of 200 kilodaltons (kDa). In some embodiments, the stabilized hemoglobin composition is concentrated by filtration into an electrolyte solution. In some embodiments, the filtration is ultrafiltration. In some embodiments, the electrolyte solution minimizes formation of Methemoglobin (MetHb). In some embodiments, the electrolyte solution comprises N-acetyl-L-cysteine. In some embodiments, the dilute hemoglobin solution comprises a hemoglobin concentration of less than 20 g/L. In some embodiments, the dilute hemoglobin solution comprises a hemoglobin concentration of 10-20 g/L. In some embodiments, the stabilized hemoglobin composition comprises: less than 5% MetHb, optionally less than 1% MetHb; and/or less than 10% hemoglobin dimers, optionally less than 5% hemoglobin dimers. In some embodiments, the stabilized hemoglobin composition comprises at least 20% tetrameric hemoglobin, optionally at least 25% tetrameric hemoglobin, and/or at least 60% greater-than-tetrameric molecular weight hemoglobin oligomers, optionally at least 70% greater-than-tetrameric molecular weight hemoglobin oligomers. In some embodiments, the stabilized hemoglobin composition comprises: 20-35% of the total hemoglobin being in tetrameric form; 15-20% of the total hemoglobin being in octameric form; 40-55% of the total hemoglobin being in greater-than-octameric form; less than 5% of the total hemoglobin being in dimer form; or any combination thereof. In some embodiments, the stabilized hemoglobin is stabilized by contacting at least one stabilizing agent selected from a group consisting of: glutaraldehyde, succindialdehyde, activated forms of polyoxyethylene and dextran, α-hydroxy aldehydes, glycolaldehyde, N-maleimido-6-aminocaproyl-(2′-nitro, 4′-sulfonic acid)-phenyl ester, m-maleimidobenzoic acid-N-hydroxysuccinimide ester, succinimidyl 4-(N-maleimidomethyl)cyclohexane-1-carboxylate, sulfosuccinimidyl 4-(N-maleimidomethyl) cyclohexane-1-carboxylate, m-maleimidobenzoyl-N-hydroxysuccinimide ester, m-maleimidobenzoyl-N-hydroxysulfosuccinimide ester, N-succinimidyl(4-iodoacetyl)aminobenzoate, sulfosuccinimidyl(4-iodoacetyl)aminobenzoate, succinimidyl 4-(p-maleimidophenyl) butyrate, sulfosuccinimidyl 4-(p-maleimidophenyl)butyrate, 1-ethyl-3-(3-dimethylaminopropyl)carbodiimide hydrochloride, N,N′-phenylene dimaleimide, a bis-imidate compound, an acyl diazide compound, an aryl dihalide compound, and combinations thereof.

In some embodiments, the stabilized hemoglobin has a longer half-life than non-stabilized or oxygenated hemoglobin and minimizes breakdown of tetrameric hemoglobin into dimers that cause renal toxicity. In some embodiments, the stabilized hemoglobin comprises at least one subunit that is synthesized in vitro. In some embodiments, the at least one subunit comprises a gamma (γ) subunit.

In some embodiments, the stabilized hemoglobin composition is manufactured in a single use fashion. In some embodiments, the single use fashion comprises using closed, pre-sterilized, single use systems; single use product contact materials; and/or single use ultra-low density polyethylene bags. In some embodiments, manufacturing the stabilized hemoglobin composition in a single use fashion limits additional exposure to endotoxins and limits or eliminates the need for NaOH purging of the manufacturing systems.

In another aspect, the present disclosure provides a system for manufacturing a stabilized hemoglobin solution comprising the means to carry out a method according to any one of the foregoing embodiments.

### BRIEF DESCRIPTION OF THE SEVERAL VIEWS OF THE DRAWINGS

FIG. 1 is an image of a fluid (e.g., blood) from which purified hemoglobin can be obtained.

FIG. 2 is a schematic of a cell washing process step for purification of proteins (e.g., hemoglobin) from a fluid.

FIG. 3 is a schematic of a cell lysis process for purification of protein (e.g., hemoglobin) solution.

FIG. 4 is a schematic of a process for deoxygenation and filtration of a protein (e.g., hemoglobin) solution.

FIG. 5 is a schematic of an anion exchange chromatography purification process for filtration of a protein (e.g., hemoglobin) solution

FIG. 6A-FIG. 6B are schematics of a protein (e.g., hemoglobin) deoxygenation process. FIG. 6A is a schematic of a concentration and deoxygenation system for the first step of protein solution deoxygenation. FIG. 6B is a schematic of a buffer exchange and filtration system for the second step of protein solution deoxygenation.

FIG. 7 is a schematic of a polymerization process for the stabilization of a protein (e.g., hemoglobin).

FIG. 8 is a schematic of a borohydride reduction process.

FIG. 9 is a schematic depicting an alternate embodiment of a cell washing process for purification of proteins (e.g., hemoglobin) from a fluid.

FIG. 10 is a schematic depicting an alternate embodiment of a cell lysis process for purification of protein (e.g., hemoglobin) solution.

FIG. 11 is a schematic depicting an alternate embodiment of a process for deoxygenation and filtration of a protein (e.g., hemoglobin) solution.

FIG. 12 is a schematic depicting an alternate embodiment of an anion exchange chromatography purification process for filtration of a protein (e.g., hemoglobin) solution

FIG. 13A-FIG. 13B are schematics depicting alternate embodiments of a protein (e.g., hemoglobin) deoxygenation process. FIG. 13A is a schematic depicting an alternate embodiment of a concentration and deoxygenation system for the first step of protein solution deoxygenation.

FIG. 13B is a schematic depicting an alternate embodiment of a buffer exchange and filtration system for the second step of protein solution deoxygenation.

FIG. 14 is a schematic depicting an alternate embodiment of a polymerization process for the stabilization of a protein (e.g., hemoglobin).

FIG. 15 is a schematic depicting an alternate embodiment of a borohydride reduction process.

FIG. 16 is a schematic depicting a sterile filtration process for a protein (e.g., hemoglobin) solution.

FIG. 17A-FIG. 17B are images of devices for cell recovery or centrate clarification (e.g., CARR Centritech's UniFuge). FIG. 17A shows an image of a device for cell recovery or centrate clarification. FIG. 17B shows an image of a device for cell recovery or centrate clarification.

FIG. 18 is an image of a separation system (e.g., CARR UniFuge Pilot Centritech Separation System) with features such as single-use disposable module, no CIP or SIP necessary, fully automated, high cell recovery rates, mammalian and insect cell processing potential, integrated trolley, intuitive software, low shear processing, and minimal reduction in viability of recovered cells. Device may be created in state-of-the-art manufacturing facility.

FIG. 19A-FIG. 19B are images of a separation chamber (e.g., UniFuge single use “GR-AC” separation chamber) with features such as glass-reinforced feed and centrate tubes, advanced core with vane accelerator flange, and 0.2″ clearance. Specifications for the device include feed flow range of 0.1-4.0 per minute. FIG. 19A is a perspective view of a separation chamber (e.g., UniFuge single use “GR-AC” separation chamber). FIG. 19B is a top view of a separation chamber (e.g., UniFuge single use “GR-AC” separation chamber).

FIG. 20 is an image of a typical installation of a separation chamber and tubeset fully assembled module in a system (e.g., UniFuge system).

FIG. 21 is an image of a separation chamber and tubeset fully assembled (e.g., UniFuge single use “GR-AC” module) with features such as 4-pinch valve configuration, glass-reinforced feedtube and centrate tube, advanced core with vane accelerator flange 0.2″ clearance, includes Meissner filter and tubeset with 24″/18″ C-flex. Feed flow range may be 0.1-4.0 L per minute.

FIG. 22 is a series of images of a tubeset assembly (e.g., UniFuge tubeset assembly) with features such as 4-pinch valve with Meissner filter, 24″ long ⅜″ I.D. C-flex connection tubes. The tubeset assembly uses item a-item u. Item a is a ½″ ID×¾″ OD tubing pharmed 36.00″ OAL that may be part number (no.) P003. Item b is a ½″ WYE connector polypro that may be part no. P006. Item c is a ½″ ID×¾″ OD tubing platinum cured silicone 36.00″ OAL that may be part no. P002. Item d is a ½″ straight connector, polypro that may be part no. P005. Item e is a ½″ ID×¾″ OD tubing 37 C-flex 24.00″ OAL that may be part no. P004. Item f is a ½″ tube plug polypro that may be part no. P007. Item g is a large tubing clamp poly that may be part no. P027. Item h is yellow tape that may be part no. P076. Item i is green tape that may be part no. P075. Item j is a ½″ ID×¾″ OD tubing platinum cured silicone 6.00″ OAL that may be part no. P002. Item k is a ½″ pressure sensor polycarbonate that may be part no. P009. Item 1 is a 3/16″ ID× 3/16″ OD tubing platinum cured silicone 18.00″ OAL that may be part no. P015. Item m is a 3/16″ ID Meissner HB 0.2 steridyne filter, CFVMV 0.2-33A1 that may be part no. P016. Item n is a 3/16″ ID× 5/16″ OD tubing platinum cured silicone 4.00″ OAL that may be part no. P0015. Item o is a MIN cable tie used for ¾″- 5/16″ ID tubing that may be part no. P063. Item p is a STD cable tie used for ⅜″ and above ID tubing that may be part no. P062. Item q is blue tape that may be part no. P074. Item r is white tape that may be part no. P080. Item s is a ½″×⅜″ reducer polypro that may be part no. P052. Item t is a ⅜″ ID×⅚″ OD tubing 37 C-flex 18.00″ OAL that may be part no. P050. Item u is a ⅜″ tube plug plypro that may be part no. P053.

FIG. 23A-FIG. 23B show two views of a device that may be employed in conjunction with a protein purification system as described herein. FIG. 23A shows one view of the device. FIG. 23B shows another view of the device.

FIG. 24 is an image of a Millipore Clarisolve 60HX or like device for blood depth filtration (60 μm and 0.027 m²/0.29 ft²).

FIG. 25 is an image of a Millipore Clarisolve 60HX or like device connected to an assembly for blood depth filtration.

FIG. 26 is a chart depicting an example of protein cross-linking distribution for polymerization step data.

FIG. 27 is a series of graphs depicting protein cross-linking distribution polymerization step data. Various protein peaks at different stages of cross-linking are displayed.

FIG. 28 is an image of polymerization step assembly. Different glutaraldehyde/bHB proportions and types of manifold were tested. Three polymerization reactions were performed on 2 days to evaluate reproducibility with the optimized manifold. Testing parameters included 1 lot on 04 may and 2 lots on 5 May with 18 g of material per test and 29 mg glutaraldehyde per gram of hemoglobin (bHB). Testing apparatus in FIG. 28 has a static mixer 3/16″ OD×4 cm length, a T-shaped connector instead of Y-shaped to avoid Glut reflux, valves on retentate tubing for closed system conc./diaf., and continuous N2 sparging.

FIG. 29 is a schematic depicting another embodiment of a polymerization process set up.

FIG. 30 is a series of graphs and images depicting C800 QEX (or equivalent) chromatography gradient optimization 1. Gradient optimization 2 resulted in significant improvement in removal of major 30 KDa impurity along with 75% yield. Loading more than 163 mg bHB/ml resin may be possible.

FIG. 31 is a chart depicting technical specifications for C800 QEX (or equivalent) chromatography gradient optimization 1.

FIG. 32 is a series of graphs and images depicting C800 QEX (or equivalent) chromatography gradient optimization 2. Gradient optimization 2 has a slower gradient and higher protein load compared to optimization 1 (FIG. 30 and FIG. 3). Gradient optimization 2 had a slight amount of bHB in the FT, 80% yield, good efficacy of CIP method (lx), good resolution, and good recovery at 236 mg bHB/ml resin.

FIG. 33 is a chart depicting technical specifications for C800 QEX (or equivalent) chromatography gradient optimization 2.

FIG. 34 is a flow chart depicting C800 QEX (or equivalent) chromatography optimization of CIP of Q sepharose XL.

FIG. 35 is an image of an assembly for C800 QEX chromatography (or equivalent). This image depicts an assembly and process with 412 ml column (5 cm diameter), 180-220 mg bHB/ml resin, three runs to process C500 1705A, fraction collector to be used for first runs, buffers will be continuously N2 sparged, and a fraction collector that will be wrapped in an atmosbag inflated with N2. This gradient method was optimized in April on 2.6 cm diameter column.

FIG. 36 is a series of images depicting storage of C500. The product can be stored at 4° C. for up to 4 weeks. Product is bottle sealed in atmosbag filled with N2 after 3 cycles of vaccum-N2.

FIG. 37A-FIG. 37E are a series of charts, graphs, and images depicting 10 kDa diafiltration. FIG. 37A is a chart depicting data regarding 10 kDa diafiltration. FIG. 37B is a plot depicting permeate volume (L) and Flux (LMH) for C5001705A 10 kDa diafiltration. FIG. 37C is a plot depicting TMP and Flux (LMH) for C5001705A 10 kDa diafiltration. FIG. 37D is a schematic of the 10 kDa diafiltration process. FIG. 37E is an image of the 10 kDa diafiltration apparatus. Despite the slight red coloration of the permeate, no bHB was detected by cooximeter. Retentate was filtered by Sartopore 2 sterile MidiCap 0.45 μm+0.2 μm filter.

FIG. 38A-FIG. 38C are a series of charts and graphs depicting 100 kDa diafiltration. FIG. 38A is a plot of Permeate volume (L) and Permeate bHB concentration (g/dL) for 100 kDa diafiltration. Less than 1% of bHB was measured in the retentate by cooximeter after diafiltration (1.7 g/247 g). FIG. 38B is plot of permeate volume (L) and retentate total bHB (%) for 100 kDa diafiltration. FIG. 38C is a chart depicting data from 100 kDa diafiltration process.

FIG. 39 is a series of images of the assembly for the 100 kDa diafiltration process.

FIG. 40 is a schematic of the 100 kDa diafiltration process. The diafiltration process involves (1) Constant N2 sparging of retentate, permeate, and diafiltration buffer (H20) (2) diafiltration H₂O is MilliQ H₂O at <0.005 EU/ml diafiltered with 10 kDa membrane (3) Addition of diafiltration buffer is performed through a T fitting with a static mixer directly in the retentate tube to improve the homogeneity of the retentate without using magnetic stirrer. (4) Permeate flow control with peristaltic pump to prevent formation of gel layer and flux reduction and to bridge with large pilot scale. (5) Brief passage of the feed through 40° C. heat exchanger before entering the membrane which promotes increase in the proportion of the transient dimeric bHB form to improve diafiltration efficacy and yield.

FIG. 41 is a schematic depicting hollow fiber next batch blood wash. A 0.65 μm hollow fiber will be available for next batch. The set up will include permeate flow control.

FIG. 42A-FIG. 42C are a series of images and charts depicting blood wash and lysis. FIG. 42A is a chart depicting data for blood wash and lysis processes. FIG. 42B is an image of the blood wash and lysis apparatus. FIG. 42C is a more complete image of the blood wash and lysis process apparatus. For the wash a hollow fiber cartridge was not available. Red cells are washed by centrifugation. Blood is diluted 1:1 in Citrate saline (CSB) and centrifuged. Cell pellet is resuspended in CSB and centrifuged three times (total of four centrifugations). For the lysis a 1:1 dilution in H₂O with static mixing. Centrifugation 14000×g to remove cell debris.

FIG. 43 depicts a commercial scale manufacturing facility employing exemplary systems and methods described herein.

### DETAILED DESCRIPTION

The present disclosure provides methods and systems for the production of stabilized hemoglobin solutions with remarkably low endotoxin content. The stabilized hemoglobin solution is a monomeric mammalian hemoglobin in cross-linked form, substantially free of endotoxins, phospholipids and non-hemoglobin proteins such as enzymes. The stabilized hemoglobin may also be highly concentrated and deoxygenated.

### Definitions

Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term “about.” Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it is understood that the particular value forms another aspect. It is further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. It is also understood that throughout the application, data are provided in a number of different formats and that this data represent endpoints and starting points and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed. Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

By “agent” is meant any small protein based or other compound, antibody, nucleic acid molecule, or polypeptide, or fragments thereof.

By “alteration” is meant a change (increase or decrease) in the molecular weight distribution of a hemoglobin solution stabilized using a stabilization technique or reaction, as detected by standard art-known methods such as those described herein. As used herein, an alteration includes a 5% change in crosslinked levels, e.g., a 5% to 95%, or 100% change in cross-linked molecular stabilization levels. In some embodiments, an alteration includes at least a 5% change, at least a 10% change in protein stabilization, a 25% change, an 80% change, a 100% change, a 200% change, a 300% change, a 400% change, a 500% change, a 600% change in protein stabilization and/or stable molecular size.

By “ameliorate” is meant decrease, suppress, attenuate, diminish, arrest, or stabilize the development or progression of a disease.

The term “antibody” (Ab) as used herein includes monoclonal antibodies, polyclonal antibodies, multispecific antibodies (e.g., bispecific antibodies), and antibody fragments, so long as they exhibit the desired biological activity. The term “immunoglobulin” (Ig) is used interchangeably with “antibody” herein.

By “binding to” a molecule is meant having a physicochemical affinity for that molecule.

The term “blood substitute” or “hemoglobin-based oxygen carrier” or “HBOC” is intended to be a material having the ability to transport and supply oxygen to vital organs and tissues and to maintain intravascular oncotic pressure. Accordingly, the term encompasses materials known in the art as “plasma expanders” and “resuscitation fluids” as well.

By “control” or “reference” is meant a standard of comparison. In one aspect, as used herein, “changed as compared to a control” sample or subject is understood as having a level that is statistically different than a sample from a normal, untreated, or control sample. Control samples include, for example, cells in culture, one or more laboratory test animals, or one or more human subjects. Methods to select and test control samples are within the ability of those in the art. An analyte can be a naturally occurring substance that is characteristically expressed or produced by the cell or organism (e.g., an antibody, a protein) or a substance produced by a reacting substance to form a covalent bond (e.g., glutaraldehyde). Depending on the method used for detection, the amount and measurement of the change can vary. Determination of statistical significance is within the ability of those skilled in the art, e.g., the number of standard deviations from the mean that constitute a positive result.

The term “cross-linked” or “polymerized” is intended to encompass both inter-molecular and intramolecular polyhemoglobin, with at least 50% of the polyhemoglobin of greater than tetrameric form.

“Detect” refers to identifying the presence, absence, or amount of the agent (e.g., a nucleic acid molecule, for example deoxyribonucleic acid (DNA) or ribonucleic acid (RNA)) to be detected.

By “detectable label” is meant a composition that when linked (e.g., joined—directly or indirectly) to a molecule of interest renders the latter detectable, via, for example, spectroscopic, photochemical, biochemical, immunochemical, or chemical means. Direct labeling can occur through bonds or interactions that link the label to the molecule, and indirect labeling can occur through the use of a linker or bridging moiety which is either directly or indirectly labeled. A “detection step” may use any of a variety of known methods to detect the presence of nucleic acid (e.g., methylated DNA) or polypeptide. The types of detection methods in which probes can be used include Western blots, Southern blots, dot or slot blots, and Northern blots.

By the terms “effective amount” and “therapeutically effective amount” of a formulation or formulation component is meant a sufficient amount of the formulation or component, alone or in a combination, to provide the desired effect. For example, by “an effective amount” is meant an amount of a compound, alone or in a combination, required to ameliorate the symptoms of an anemic and or iron deficient state, e.g., hypoxia, relative to an untreated patient. The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

By the term “endotoxin(s)” is intended the generally cell-bound lipopolysaccharides produced as a part of the outer layer of bacterial cell walls, which under many conditions are toxic. When injected into an animal, endotoxins cause fever, diarrhea, hemorrhagic shock, and other tissue damage.

By the term “endotoxin unit” (EU) is intended that meaning given by the United States Pharmacopeial Convention of 1983, Page 3014, which defined EU as the activity contained in 0.2 nanograms of the U.S. reference standard lot EC-2. One vial of EC-2 contains 5,000 EU.

By “fragment” is meant a portion of a protein molecule. This portion contains, preferably, at least the heme iron portion of the molecule or original protein construct of hemoglobin. For example, a fragment may contain 1, 2 or 4 side chains of the alpha and beta fragments of the native hemoglobin molecule. However, the invention also comprises protein fragments, so long as they exhibit the desired biological activity from the full length globular protein structure. For example, illustrative poly-amino acid segments with total weights of about 16 kDa, about 32 kDa, in size (including all intermediate weights) are included in many implementations of this invention. Similarly, a protein fragment of almost any length is employed if it is the iron carrier (heme group).

“Hemoglobin” or “Hb” is the protein molecule in red blood cells that carries oxygen from the lungs to the body's tissues and returns carbon dioxide from the tissues back to the lungs. Hemoglobin is typically composed of four globulin chains. The normal adult hemoglobin molecule contains two alpha-globulin chains and two beta-globulin chains. In fetuses and infants, beta chains are not common and the hemoglobin molecule is made up of two alpha chains and two gamma chains. Each globulin chain contains an important iron-containing porphyrin compound termed heme. Embedded within the heme compound is an iron atom that is vital in transporting oxygen and carbon dioxide in our blood. The iron contained in hemoglobin is also responsible for the red color of blood.

The terms “isolated,” “purified,” or “biologically pure” refer to material that is free to varying degrees from components which normally accompany it as found in its native environment. “Isolate” denotes a degree of separation from original source or surroundings. “Purify” denotes a degree of separation that is higher than isolation.

The term “immobilized” or “attached” refers to a probe (e.g., nucleic acid or protein) and a solid support in which the binding between the probe and the solid support is sufficient to be stable under conditions of binding, washing, analysis, and removal. The binding may be covalent or non-covalent. Covalent bonds may be formed directly between the probe and the solid support or may be formed by a cross linker or by inclusion of a specific reactive group on either the solid support or the probe or both molecules. Non-covalent binding may be one or more of electrostatic, hydrophilic, and hydrophobic interactions. Included in non-covalent binding is the covalent attachment of a molecule to the support and the non-covalent binding of a biotinylated probe to the molecule. Immobilization may also involve a combination of covalent and non-covalent interactions.

By an “isolated polypeptide” is meant a polypeptide of the invention that has been separated from components that naturally accompany it. Typically, the polypeptide is isolated when it is at least 60%, by weight, free from the proteins and naturally-occurring organic molecules with which it is naturally associated. Preferably, the preparation is at least 75%, more preferably at least 90%, and most preferably at least 99%, by weight, a polypeptide of the invention. An isolated polypeptide fraction and or protein of the invention may be obtained, for example, by extraction from a natural source, by expression of a recombinant nucleic acid encoding such a material; or by chemically synthesizing the protein. Purity can be measured by any appropriate method, for example, column chromatography, polyacrylamide gel electrophoresis, or by HPLC analysis.

By “marker” is meant any protein or polynucleotide having an alteration in expression level or activity that is associated with a disease or disorder, e.g., neoplasia.

“Methemoglobin” or “methaemoglobin” is a hemoglobin in the form of metalloprotein, in which the iron in the heme group is in the Fe³⁺ (ferric) state, not the Fe′ (ferrous) of normal hemoglobin. Methemoglobin cannot bind oxygen, which means it cannot carry oxygen to tissues. In human blood, a trace amount of methemoglobin is normally produced spontaneously, but when present in excess the blood becomes abnormally dark bluish brown. The NADH-dependent enzyme methemoglobin reductase (a type of diaphorase) is responsible for converting methemoglobin back to hemoglobin. Normally one to two percent of a person's hemoglobin is methemoglobin; a higher percentage than this can be genetic or caused by exposure to various chemicals and depending on the level can cause health problems known as methemoglobinemia. An abnormal increase of methemoglobin will increase the oxygen binding affinity of normal hemoglobin, resulting in a decreased unloading of oxygen to the tissues and possible tissue hypoxia.

By “modulate” is meant alter (increase or decrease). Such alterations are detected by standard art-known methods such as those described herein.

By “neoplasia” is meant a disease or disorder characterized by excess proliferation or reduced apoptosis. Illustrative neoplasms for which the invention can be used include, but are not limited to pancreatic cancer, leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (Hodgkin's disease, non-Hodgkin's disease), Waldenstrom's macroglobulinemia, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, nile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, glioblastoma multiforme, astrocytoma, medulloblastoma, craniopharyngioma, ependymoma, pinealoma, hemangioblastoma, acoustic neuroma, oligodenroglioma, schwannoma, meningioma, melanoma, neuroblastoma, and retinoblastoma).

The term, “normal amount” refers to a normal amount of a complex in an individual known not to be diagnosed with cancer or various metabolic and physiologic disease states. The amount of a given molecule can be measured in a test sample and compared to the “normal control level,” utilizing techniques such as reference limits, discrimination limits, or risk defining thresholds to define cutoff points and abnormal values (e.g., for neoplasia, hypoxia, ischemia). The “normal control level” means the level of one or more proteins (or nucleic acids) or combined protein indices (or combined nucleic acid indices) typically found in a subject known not to be suffering from cancer or a physiologic oxygen deficient status. Such normal control levels and cutoff points may vary based on whether a molecule is used alone or in a formula combining other proteins into an index. Alternatively, the normal control level can be a database of protein patterns from previously tested subjects who did not develop cancer or other relevant diseases over a clinically relevant time horizon. In another aspect, the normal control level can be a level relative to a regular cellular function and the level of oxygenation. The level that is determined may be the same as a control level or a cut off level or a threshold level, or may be increased or decreased relative to a control level or a cut off level or a threshold level. In some aspects, the control subject is a matched control of the same species, gender, ethnicity, age group, smoking status, body mass index (BMI), current therapeutic regimen status, medical history, or a combination thereof, but differs from the subject being diagnosed and assessed in that the control does not suffer from the disease in question or is not at risk for the disease or reflects signs and symptoms of oxygen deprivation.

Relative to a control level, the level that is determined may be an increased level. As used herein, the term “increased” with respect to level (e.g., hemoglobin level, oxygenation level, expression level, biological activity level, etc.) refers to any % increase above a control level. The increased level may be at least or about a 5% increase, at least or about a 10% increase, at least or about a 15% increase, at least or about a 20% increase, at least or about a 25% increase, at least or about a 30% increase, at least or about a 35% increase, at least or about a 40% increase, at least or about a 45% increase, at least or about a 50% increase, at least or about a 55% increase, at least or about a 60% increase, at least or about a 65% increase, at least or about a 70% increase, at least or about a 75% increase, at least or about a 80% increase, at least or about a 85% increase, at least or about a 90% increase, or at least or about a 95% increase, relative to a control level. In some embodiments, the increased level may be more than 100%, 150%, 200%, 250%, 300%, 350%, 400%, 450%, or 500% increased.

Relative to a control level, the level that is determined may be a decreased level. As used herein, the term “decreased” with respect to level (e.g., hemoglobin level, oxygenation level, expression level, biological activity level, etc.) refers to any % decrease below a control level. The decreased level may be at least or about a 1% decrease, at least or about a 5% decrease, at least or about a 10% decrease, at least or about a 15% decrease, at least or about a 20% decrease, at least or about a 25% decrease, at least or about a 30% decrease, at least or about a 35% decrease, at least or about a 40% decrease, at least or about a 45% decrease, at least or about a 50% decrease, at least or about a 55% decrease, at least or about a 60% decrease, at least or about a 65% decrease, at least or about a 70% decrease, at least or about a 75% decrease, at least or about a 80% decrease, at least or about a 85%) decrease, at least or about a 90% decrease, or at least or about a 95% decrease, relative to a control level.

As used herein, “obtaining” as in “obtaining an agent” includes synthesizing, purchasing, or otherwise acquiring the agent.

Unless specifically stated or obvious from context, as used herein, the term “or” is understood to be inclusive.

“Oxyhemoglobin” or “oxyhaemoglobin” is the oxygen-loaded form of hemoglobin. In general, hemoglobin can be saturated with oxygen molecules (oxyhemoglobin), or desaturated with oxygen molecules (deoxyhemoglobin). Oxyhemoglobin is formed during physiological respiration when oxygen binds to the heme component of hemoglobin in red blood cells. This process occurs in the pulmonary capillaries adjacent to the alveoli of the lungs. The oxygen then travels through the blood stream to be dropped off at cells where it is utilized as a terminal electron acceptor in the production of ATP by the process of oxidative phosphorylation.

The phrase “pharmaceutically acceptable carrier” is art recognized and includes a pharmaceutically acceptable material, composition or vehicle, suitable for administering compounds of the present invention to mammals. The carriers include liquid or solid filler, diluent, excipient, solvent or encapsulating material, involved in carrying or transporting the subject agent from one organ, or portion of the body, to another organ, or portion of the body. Each carrier must be “acceptable” in the sense of being compatible with the other ingredients of the formulation and not injurious to the patient. Some examples of materials which can serve as pharmaceutically acceptable carriers include: sugars, such as lactose, glucose and sucrose; gelatin; excipients; pyrogen-free water; isotonic saline; Ringer's solution; ethyl alcohol; phosphate buffer solutions; and other non-toxic compatible substances employed in pharmaceutical formulations.

The terms “preventing” and “prevention” refer to the administration of an agent or composition to a clinically asymptomatic individual who is at risk of developing, susceptible, or predisposed to a particular adverse condition, disorder, or disease, and thus relates to the prevention of the occurrence of symptoms and/or their underlying cause.

“Primers” and “primer sets” refer to oligonucleotides that may be used, for example, for PCR. A primer set may comprise at least 2, 4, 6, 8, 10, 12, 14, 16, 18, 20, 30, 40, 50, 60, 80, 100, 200, 250, 300, 400, 500, 600, or more primers.

By “protein” or “polypeptide” or “peptide” is meant any chain of more than two natural or unnatural amino acids, regardless of post-translational modification (e.g., glycosylation or phosphorylation), constituting all or part of a naturally-occurring or non-naturally occurring polypeptide or peptide, as is described herein.

A “purified” or “biologically pure” protein is sufficiently free of other materials such that any impurities do not materially affect the biological properties of the protein or cause other adverse consequences. That is, stabilized protein of a fragment to a polymer in this invention, it is purified if it is substantially free of cellular material, viral material, or culture medium when produced by recombinant DNA techniques, or chemical precursors or other chemicals when chemically synthesized, and all other stromal red blood cell or other blood proteins or blood components and cellular debris. Purity, homogeneity and stability are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation, glycosylation, or polymerization different modifications may give rise to different isolated proteins, which can be separately purified.

By “reduces” is meant a negative alteration of at least 10%, 25%, 50%, 75%, or 100%.

A “reference sequence” is a defined sequence used as a basis for sequence comparison or a gene expression comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, preferably at least about 20 amino acids, more preferably at least about 25 amino acids, and even more preferably about 35 amino acids, about 50 amino acids, or about 100 amino acids. For nucleic acids, the length of the reference nucleic acid sequence will generally be at least about 40 nucleotides, preferably at least about 60 nucleotides, more preferably at least about 75 nucleotides, and even more preferably about 100 nucleotides or about 300 or about 500 nucleotides or any integer thereabout or there between.

The term “sample” as used herein refers to a biological sample obtained for the purpose of evaluation in vitro. Exemplary tissue samples for the methods described herein include tissue samples from neoplasias or circulating exosomes. With regard to the methods disclosed herein, the sample or patient sample preferably may comprise any body fluid or tissue. In some embodiments, the bodily fluid includes, but is not limited to, blood, plasma, serum, lymph, breast milk, saliva, mucous, semen, vaginal secretions, cellular extracts, inflammatory fluids, cerebrospinal fluid, feces, vitreous humor, or urine obtained from the subject. In some aspects, the sample is a composite panel of at least two of a blood sample, a plasma sample, a serum sample, and a urine sample. In exemplary aspects, the sample comprises blood or a fraction thereof (e.g., plasma, serum, fraction obtained via leukopheresis). Preferred samples are whole blood, serum, plasma, or urine. A sample can also be a partially purified fraction of a tissue or bodily fluid. A reference sample can be a “normal” sample, from a donor not having the disease or condition fluid, or from a normal tissue in a subject having the disease or condition. A reference sample can also be from an untreated donor or cell culture not treated with an active agent (e.g., no treatment or administration of vehicle only). A reference sample can also be taken at a “zero time point” prior to contacting the cell or subject with the agent or therapeutic intervention to be tested or at the start of a prospective study.

A “solid support” describes a strip, a polymer, a bead, or a nanoparticle. The strip may be a nucleic acid-probe (or protein) coated porous or non-porous solid support strip comprising linking a nucleic acid probe to a carrier to prepare a conjugate and immobilizing the conjugate on a porous solid support. Well-known supports or carriers include glass, polystyrene, polypropylene, polyethylene, dextran, nylon, amylases, natural and modified celluloses, polyacrylamides, gabbros, and magnetite. The nature of the carrier can be either soluble to some extent or insoluble for the purposes of the present invention. The support material may have virtually any possible structural configuration so long as the coupled molecule is capable of binding to a binding agent (e.g., an antibody or nucleic acid molecule). Thus, the support configuration may be spherical, as in a bead, or cylindrical, as in the inside surface of a test tube, or the external surface of a rod. Alternatively, the surface may be flat such as a sheet, or test strip, etc. For example, the supports include polystyrene beads. Those skilled in the art will know many other suitable carriers for binding antibody or antigen, or will be able to ascertain the same by use of routine experimentation. In other aspects, the solid support comprises a polymer, to which an agent is chemically bound, immobilized, dispersed, or associated. A polymer support may be a network of polymers, and may be prepared in bead form (e.g., by suspension polymerization). The location of active sites introduced into a polymer support depends on the type of polymer support. For example, in a swollen-gel-bead polymer support the active sites are distributed uniformly throughout the beads, whereas in a macroporous-bead polymer support they are predominantly on the internal surfaces of the macropores. The solid support, e.g., a device contains a binding agent alone or together with a binding agent for at least one, two, three or more other molecules.

By “specifically binds” is meant a compound or antibody that recognizes and binds a polypeptide of the invention, but which does not substantially recognize and bind other molecules in a sample, for example, a biological sample, which naturally includes a polypeptide/conjugated purified protein of the invention.

The terms “stabilized hemoglobin solution” and “stabilized hemoglobin composition” refer to the disclosed compositions comprising cross-linked (i.e., stabilized) deoxygenated hemoglobin. Such solutions may be prepared in a pharmaceutical formulation and/or provided in an injection device and may be used to treat one or more anemic or hypoxic conditions.

The term “subject” as used herein includes all members of the animal kingdom prone to suffering from the indicated disorder. In some aspects, the subject is a mammal, and in some aspects, the subject is a human. The methods are also applicable to companion animals such as dogs and cats as well as livestock such as cows, horses, sheep, goats, pigs, and other domesticated and wild animals.

The term “substantially endotoxin free”, for the purposes of the present invention, may be described functionally as a stabilized hemoglobin composition which contains less than 1.0 endotoxin units per milliliter of solution, at a concentration of 10 grams of hemoglobin per deciliter of solution, though the final concentration may be between 15 and 20 grams of hemoglobin per deciliter of solution. In some embodiments, the “substantially endotoxin free” hemoglobin drug substance of the present disclosure will contain less than 0.5, and preferably less than 0.25, most preferably less than 0.02 endotoxin units per milliliter of solution (EU/ml) as measured by the Limulus Amebocytic Lysate (LAL) assay. The LAL assay is described by Nachum et al., Laboratory Medicine, 13:112-117 (1982) and Pearson III et al., Bioscience, 30:416-464 (1980), incorporated by reference herein.

The term “substantially deoxygenated” or “highly deoxygenated”, for the purposes of the present disclosure, describes a hemoglobin solution that contains less than 0.1 mg/mL of dissolved oxygen or significantly less than 0.1 mg/mL of dissolved oxygen. In some embodiments, the hemoglobin solution may contain less than 0.05 mg/mL, less than 0.04 mg/mL, less than 0.03 mg/mL, less than 0.02 mg/mL, or less than 0.01 mg/mL of dissolved oxygen.

By “substantially identical” is meant a polypeptide/protein or nucleic acid molecule exhibiting at least 80% identity to a reference amino acid sequence (for example, any one of the amino acid sequences described herein) or nucleic acid sequence (for example, any one of the nucleic acid sequences described herein). Preferably, such a sequence is at least 80%, at least 85%), at least 90%, at least 95%, or at least 99% identical at the amino acid level or nucleic acid to the sequence used for comparison.

By “substantially pure” is meant a protein or polypeptide that has been separated from the components that naturally accompany it. Typically, the proteins and polypeptides are substantially pure when they are at least 95%, or even 99%, by weight, free from the other proteins and naturally-occurring organic molecules with they are naturally associated.

A subject “suffering from or suspected of suffering from” a specific disease, condition, or syndrome has a sufficient number of risk factors or presents with a sufficient number or combination of signs or symptoms of the disease, condition, or syndrome such that a competent individual would diagnose or suspect that the subject was suffering from the disease, condition, or syndrome. Methods for identification of subjects suffering from or suspected of suffering from conditions associated with cancer is within the ability of those in the art. Subjects suffering from, and suspected of suffering from, a specific disease, condition, or syndrome are not necessarily two distinct groups.

As used herein, “susceptible to” or “prone to” or “predisposed to” or “at risk of developing” a specific disease or condition refers to an individual who based on genetic, environmental, health, and/or other risk factors is more likely to develop a disease or condition than the general population. An increase in likelihood of developing a disease may be an increase of about 10%, 20%, 50%, 100%, 150%, 200%, or more.

The terms “treating” and “treatment” as used herein refer to the administration of an agent or formulation to a clinically symptomatic individual afflicted with an adverse condition, disorder, or disease, so as to effect a reduction in severity and/or frequency of symptoms, eliminate the symptoms and/or their underlying cause, and/or facilitate improvement or remediation of damage. It will be appreciated that, although not precluded, treating a disorder or condition does not require that the disorder, condition or symptoms associated therewith be completely eliminated.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All published foreign patents and patent applications cited herein are incorporated herein by reference. All other published references, documents, manuscripts and scientific literature cited herein are incorporated herein by reference. In the case of conflict, the present specification, including definitions, will control. In addition, the materials, methods, and examples are illustrative only and not intended to be limiting.

### Methods for the Manufacture of Stabilized Hemoglobin Solutions

The present disclosure relates to methods and systems for the formulation of stabilized hemoglobin solutions. Without wishing to be bound by theory, it is theorized that existing methods for producing hemoglobin-based oxygen carriers and blood substitutes have been insufficiently safe because they result in an excess of oxygenation and/or endotoxin content in the resulting compositions, which can lead to undesirable and sometimes dangerous side effects in part due to the high oxygen content of the hemoglobin comprised by such solutions. However, it has historically proven difficult to deoxygenate concentrated hemoglobin solutions and to acquire endotoxin levels within safe limits. The present application is the first to disclose methods and systems for producing such predominantly deoxygenated, stabilized hemoglobin solutions produced in a substantially endotoxin-free fashion through the use of single use systems and equipment. The following subsections provide exemplary methods for obtaining and producing the stabilized hemoglobin solutions of the present disclosure and provide exemplary characteristics of such methods and systems. Additional methods and systems useful for the present disclosure may be found in International Publication No. WO 2019/055489, the contents of which are hereby incorporated by reference in their entirety.

Generally, in some embodiments, the stabilized hemoglobin composition is prepared from a mammalian blood fraction by a process comprising 1) separation of red blood cells from the mammalian blood fraction; 2) hemolysis of the red blood cells to produce a composite of monomeric hemoglobin and stroma; 3) separation by filtration of the hemoglobin; 4) purification of the monomeric hemoglobin by high performance liquid chromatography (HPLC) to separate the hemoglobin from all other proteins residual of the red blood cells, as well as the phospholipid, enzyme and endotoxin contaminants; 5) deoxygenation and diafiltration; 6) cross-linking (polymerizing or aggregating) the monomeric hemoglobin; and/or 7) concentrating the stabilized hemoglobin solution.

In some embodiments, the process may comprise the steps of (1) obtaining the blood raw product, (2) fractionating the blood raw product to produce a red blood cell fraction which is substantially free from white blood cells and platelets, (3) mechanically disrupting the red blood cell fraction to produce a hemoglobin-containing solution, (4) clarifying the hemoglobin-containing solution to produce a hemoglobin solution which is substantially free of cellular debris, (5) microporously filtering the hemoglobin solution which is substantially free of cellular debris to produce a partially sterilized hemoglobin-containing solution, (6) ultrafiltering the partially sterilized hemoglobin-containing solution to produce a size-separated hemoglobin-containing solution, (7) chromatographically separating the size-separated hemoglobin-containing solution to produce a hemoglobin substantially free of phospholipids, non-hemoglobin proteins, and endotoxins, (8) deoxygenating the substantially endotoxin-free hemoglobin to produce a substantially deoxygenated hemoglobin solution, (9) cross-linking said substantially deoxygenated hemoglobin solution to produce stabilized hemoglobin solution, and/or (10) concentrating the stabilized hemoglobin solution, all steps done in a substantially endotoxin-free environment.

In some embodiments, the process may comprise a step after the cross-linking step to separate or partially separate monomeric and low molecular weight species of hemoglobin from the higher molecular weight polymers formed during cross-linking. In some embodiments, the process also comprises a step of concentrating the stabilized, deoxygenated hemoglobin solution to a concentration between 150 g/L and 200 g/L (inclusive of end points) of hemoglobin in solution.

In some embodiments, the process may comprise the addition of in vitro synthesized hemoglobin at any stage prior to cross-linking. In some embodiments, the process comprises formulating highly concentrated, deoxygenated, stabilized hemoglobin from a synthetic source.

In some embodiments, the process may comprise conducting any one or more of the above steps under conditions which result in a product which is substantially free of endotoxins, phospholipids and non-hemoglobin proteins such as enzymes, and has a defined molecular weight distribution of greater than about 90% between 68,000 daltons and 500,000 daltons.

Additional details and embodiments of the disclosed methods are described further in the following sections.

Hemoglobin and/or Red Blood Cell Source

More than 99% of the cells in blood are red blood cells. The major function of red blood cells is to transport hemoglobin, which in turn carries oxygen from lungs to the tissues and C02 from the tissues to the lungs. Normal red blood cells contain approximately 34 grams of hemoglobin per 100 ml of cells. Each gram of hemoglobin is capable of combining with approximately 1.33 ml of oxygen. In bovine blood the concentration of hemoglobin (bHB) in g/dL is 10.1 and with a volume of 2.96 L of blood this amounts to 299 g of bHB. Thus, bovine blood is a viable option for large-scale hemoglobin recovery.

The hemoglobin comprised in the stabilized hemoglobin compositions of the present disclosure may be obtained from an organism or may be synthetically formulated.

In some embodiments, the hemoglobin is obtained from an erythrocyte (red blood cell) source. In some embodiments, the hemoglobin is derived from a human source. In some embodiments, the hemoglobin comprises hemoglobin isolated or derived from a human, a human cell, or a human cell line. In some embodiments, the red blood cells may be from freshly drawn human blood, expired blood from blood banks (i.e., donated blood that has exceeded its shelf life), placentas, or packed erythrocytes obtained from human donor centers. In some embodiments, the stabilized hemoglobin is not isolated from a human fetus.

In some embodiments, the stabilized hemoglobin solution comprises hemoglobin isolated or derived from a non-human animal, a non-human cell or a non-human cell line. In some embodiments, the non-human animal is a live animal or a freshly slaughtered animal. In some embodiments, stabilized hemoglobin solutions may comprise hemoglobin derived or isolated from a non-human animal that is a non-human vertebrate, a non-human primate, a cetacean, a mammal, a reptile, a bird, an amphibian, or a fish. In some embodiments, red blood cells obtained from animal blood are used. In some embodiments, the hemoglobin is derived from a nonhuman mammalian blood source. Blood from a variety of sources such as bovine, ovine, or porcine may be used. Because of its ready availability, in some embodiments, bovine blood may be used. In some embodiments, the hemoglobin is derived from a bovine blood source.

In some embodiments, stabilized hemoglobin solutions may comprise hemoglobin derived or isolated from a non-human animal that is a mustelid, a captive mustelid, a rodent, a captive rodent, a raptor, or a captive bird. In some embodiments, the captive bird is of the order Psittaciformes, Passeriformes, or Columbiformes. In some embodiments, the non-human animal is not a squab that is raised for food.

In some embodiments, the stabilized hemoglobin solution may comprise hemoglobin that is partially or wholly synthetic. In some embodiments, the stabilized hemoglobin solution may comprise at least one subunit that is synthesized in vitro. In some embodiments, the stabilized hemoglobin solution may comprise at least one synthetic subunit comprising a gamma (γ) subunit.

### Red Blood Cell Collection

In some embodiments, the present stabilized hemoglobin solutions may comprise hemoglobin that is derived or isolated from red blood cells collected from a non-human animal source. For collection of red blood cells from, e.g., bovine sources, collection trochars may be used to extract the blood in a sterile manner. The trochars are carefully inserted and handled and are connected to tubing approximately 2 feet in length. In order to insert the trochar, the hide is cut away and peeled back, and the trochar is then inserted in the animal's major vessels close to the heart with care not to puncture the esophagus. Avoiding the introduction of bacteria and the maintenance of endotoxin-free of low endotoxin level material is important. This may be accomplished using individual containers that are pre-charged with an anticoagulant and that are depyrogenated and re-checked for endotoxins. Typical anticoagulants include sodium citrate. In all cases, endotoxin levels of the containers must be less than 0.01 endotoxin units as detected by LAL. In some embodiments, the red blood cells are collected via venipuncture. In some embodiments, the volume of collected blood from a single animal may be 50 mL-40 L. In some embodiments, blood is drawn from a single animal. In some embodiments, blood is drawn from more than one animal.

During or after collection, the collected blood may be treated so as to prevent coagulation. In some embodiments, the collecting vessel may be treated with an anticoagulant. In some embodiments, the collected blood may be defibrinated or citrated. Defibrinated blood is blood from which fibrin has been removed or which has been treated to denature fibrinogen without causing cell lysis. Citrated blood is blood that has been treated with sodium citrate or citric acid to prevent coagulation.

The red blood cell solution may be distributed to small vessels that can hold between 2 to 10 gallons of gathered blood in a sterile manner and, therefore, maintain the blood in an endotoxin-free state. The collected blood in its container may be capped off immediately to avoid exposure to the environment. Upon completion of the collection process, the material is chilled, typically to about 4° C., to limit bacterial growth. There is no pooling of blood at this time; the blood is later checked for endotoxins and sterility to ensure that (1) no one cow is sick; or (2) a bad collection technique has not contaminated the entire batch or collection for that day.

Additional methods for collecting blood are set forth in, e.g., U.S. Pat. Nos. 5,084,558 and 5,296,465, the contents of which are incorporated by reference in their entirety. The illustrative collection methods described in the foregoing section are not meant to be limiting, as there are many collection methods which are suitable and available to one with ordinary skill in the art.

### Red Blood Cell Defibrination

The methods and systems herein may also provide a step to defibrinate collected blood. Defibrinating the blood sets off the clotting cascade to artificially remove the fibrin molecules involved in the formation of blood clots. Defibrination can be induced by chemical or mechanical means. Chemical coagulating agents are defined herein as substances that induce clotting. For example, collagen induces coagulation so that when there is an external wound, a fibrin clot will stop blood from flowing. Artificially exposing blood to collagen will cause the formation of fibrin clots, which can be removed to produce defibrinated blood.

In some embodiments, the blood is defibrinated by exposure to a coagulating agent. Examples of coagulating agents are collagen, tissue extract, tissue factor, tissue thromboplastin, anionic phospholipid, calcium, negatively charged materials (e.g., glass, kaolin, some synthetic plastics, fabrics). A preferred clotting agent is collagen.

In some embodiments, the whole blood is exposed to the clotting agent for a period of time sufficient to cause essentially all fibrin in the blood to be converted into a fibrin clot. The appropriate time is determined by the point at which fibrin molecules apparently stop polymerizing. Chemical defibrination, defined herein as defibrination that is induced by exposure to a chemical coagulating agent, is conducted at a suitable temperature, preferably a temperature in a range of between about 4° C. and about 40° C.

In some embodiments, mechanical agitation, such as stirring, also has the effect of initiating the clotting cascade. After stirring until fibrin polymerization apparently ceases, it is possible to remove the accumulated fibrin to complete defibrination. Mechanical defibrination, defined herein as defibrination induced by agitating the blood solution, is conducted at a suitable temperature, and preferably at a temperature in a range of between about 4° C. and about 40° C.

Fibrin is then removed from the whole blood by a suitable means. An example of a suitable means is by directing the whole blood, including the fibrin, through a strainer. A mesh screen is an example of a suitable strainer. Optionally, alternatively, or in addition to the use of a strainer, cheesecloth or polypropylene filters can be employed to remove large debris, including fibrin.

In some embodiments, it is possible to defibrinate blood that has already been citrated by saturating the citrated blood with a divalent cation, and then defibrinating the solution, similar to the means by which uncitrated blood would be processed. The divalent cation may be calcium.

### Red Blood Cell Washing

In some embodiments, cell washing includes the processes of dilution and diafiltration in a continuous filtration operation. In some embodiments, a saline/citrate solution is added to the filter retentate to maintain a constant volume in the recirculation tank. The result is a reduction in the concentration of microfiltration membrane-permeable species (including membrane-permeable plasma proteins). Subsequent reconcentration of the diluted blood solution back to the original volume produces a purified blood solution.

In a preferred embodiment, the blood solution is washed by diafiltration or by a combination of discrete dilution and concentration steps with at least one solution, such as an isotonic solution, to separate red blood cells from extracellular plasma proteins, such as serum albumins or antibodies (e.g., immunoglobulins (IgG)). Preferably, the isotonic solution includes an ionic solute or is aqueous. It is understood that the red blood cells can be washed in a batch or continuous feed mode.

Acceptable isotonic solutions are known in the art and include solutions, such as a citrate/saline solution, having a pH and osmolarity which does not rupture the cell membranes of red blood cells and which displaces the plasma portion of the whole blood. The blood may be diluted to a concentration in the range between about 25% and 75% of the original concentration. A preferred isotonic solution has a neutral pH and an osmolarity between about 285-315 mOsm. In a preferred embodiment, the isotonic solution is composed of an aqueous solution of sodium citrate dihydrate (6.0 g/l) and of sodium chloride (8.0 g/l).

In one method, the whole blood is diafiltered across a membrane having a permeability limit in the range of between 0.2 μm and about 2.0 μm. Alternate suitable diafilters include microporous membranes with pore sizes that will separate RBCs from substantially smaller blood solution components, such as a 0.1 μm to 0.5 μm filter (e.g., a 0.2 μm hollow fiber filter). During cell washing, fluid components of the blood solution, such as plasma, or components which are significantly smaller in diameter than RBCs pass through the walls of the diafilter in the filtrate. Erythrocytes, platelets and larger bodies of the blood solution, such as white blood cells, are retained and mixed with isotonic solution, which is added continuously or batch-wise to form a dialyzed blood solution.

Concurrently, a filtered isotonic solution is added continuously (or in batches) to maintain volume of filtrate to compensate for the portion of the solution lost across the diafilter. In a more preferred embodiment, the volume of blood solution in the diafiltration tank is initially diluted by the addition of a volume of a filtered isotonic solution to the diafiltration tank. Preferably, the volume of isotonic solution added is about equal to the initial volume of the blood solution.

In some embodiments, the blood is washed through a series of sequential (or reverse sequential) dilution and concentration steps, wherein the blood solution is diluted by adding at least one isotonic solution, and is concentrated by flowing across a filter, thereby forming a dialyzed blood solution.

Cell washing generally is considered to be complete when the level of plasma proteins contaminating the red blood cells has been substantially reduced (typically at least about 90%). Additional washing may further separate extracellular plasma proteins from the RBCs. For instance, diafiltration with seven volumes of isotonic solution may be sufficient to remove at least about 99% of IgG from the blood solution.

### Red Blood Cell Separation and Lysis

In some embodiments, red blood cells may be further separated from other blood components, e.g., white blood cells, platelets, and the like. In some embodiments, the red blood cells are separated by centrifugation. It is understood that other methods generally known in the art for separating red blood cells from other blood components can be employed. For example, one embodiment of the invention separates red blood cells by sedimentation, wherein the separation method does not rupture the cell membranes of a significant amount of the RBCs, such as less than about 30% of the RBCs, prior to red blood cell separation from the other blood components.

In some embodiments, following purification of the red blood cells, the RBCs are lysed, resulting in the production of a hemoglobin (Hb) solution. Red blood cells may be lysed by any means that disrupt the red blood cell membrane and release hemoglobin from the interior of the cell. Methods of lysis include mechanical lysis, chemical lysis, hypotonic lysis or other known lysis methods which release hemoglobin without significantly damaging the ability of the Hb to transport and release oxygen. Means of lysing cells are known in the art and may be employed in the present methods in systems. In some embodiments, red blood cell lysis occurs via rapid change in osmotic pressure, e.g., by the addition of filtered water to the red blood cell sample.

### Hemoglobin Purification

Following lysis, the lysed red blood cell phase may be ultrafiltered to remove larger cell debris, such as proteins with a molecular weight above about 100,000 Daltons. The hemoglobin may then be separated from the non-Hb components of the filtrate.

Methods of ultrafiltration and methods of separating Hb from non-Hb components by pH gradients and chromatography are also described in U.S. Pat. No. 5,691,452, which is incorporated by reference in its entirety herein.

In some embodiments, the hemoglobin solution is purified via chromatographic means. Exemplary chromatographic methods include ion exchange chromatography, size exclusion chromatography, hydrophobic interaction chromatography, fast protein liquid chromatography, high performance liquid chromatography, and the like.

### Deoxygenation

The Hb eluate is then preferably deoxygenated prior to polymerization to form a deoxygenated Hb solution (hereinafter deoxy-Hb) for further processing into a hemoglobin-based oxygen carrier. In a preferred embodiment, deoxygenation substantially deoxygenates the Hb without significantly reducing the ability of the Hb in the Hb eluate to transport and release oxygen, such as would occur from formation of oxidized hemoglobin (metHb). Alternatively, the hemoglobin solution may be deoxygenated by chemical scavenging with a reducing agent selected from the group consisting of N-acetyl-L-cysteine (NAC), cysteine, sodium dithionite or ascorbate.

Exemplary methods of deoxygenation are also described in U.S. Pat. No. 5,895,810, which is incorporated herein by reference in its entirety.

In some embodiments, the hemoglobin solution is deoxygenated by diafiltration against a degassing membrane with nitrogen flowing across the opposite side of the membrane. In some embodiments, the hemoglobin solution is substantially diluted prior to deoxygenation. In some embodiments, the hemoglobin solution is diluted to approximately 1-20 g/L. In some embodiments, the hemoglobin solution is diluted to approximately 1, 2, 3, 4, 5, 6, 7, 8, 9, 10, 11, 12, 13, 14, 15, 16, 17, 18, 19, or 20 g/L prior to deoxygenation. In some embodiments, the hemoglobin solution is diluted to about or less than about 15 g/L prior to deoxygenation. In some embodiments, the hemoglobin solution is diluted to about 5-10 g/L, 10-15 g/L, or 15-20 g/L prior to deoxygenation. Without wishing to be bound by theory, it is believed that the significant dilution of the hemoglobin solution prior to deoxygenation results in a surprising and unexpected degree of deoxygenation previously difficult to obtain. It is also believed that the particular order of dilution, deoxygenation, and then concentration allows for the unexpected ability to concentrate the hemoglobin solution to a greater extent than previous hemoglobin solutions without complications (e.g., unacceptable levels of precipitation).

In some embodiments, the level of oxygenation in the stabilized hemoglobin solutions of the present disclosure may be measured in parts per million. In some embodiments, the level of oxygenation is substantially less than for previously disclosed hemoglobin-based oxygen carriers. In some embodiments, the level of oxy-hemoglobin in the stabilized hemoglobin solutions of the present disclosure is 50% or lower compared to the oxy-hemoglobin level of a commercially available hemoglobin based oxygen carrier, such as OxyGlobin®. In some embodiments, the level is 40% or lower. In some embodiments, the level is 30% or lower. In some embodiments, the level is 20% or lower. In some embodiments, the level is 20% or lower. In some embodiments, the level is 10% or lower. In some embodiments, the level is 5% or lower. In some embodiments, the level is 1% or lower.

### Hemoglobin Polymerization

In some embodiments, polymerization results from intramolecular cross-linking of Hb. The amount of a sulfhydryl compound mixed with the deoxy-Hb is high enough to increase intramolecular cross-linking of Hb during polymerization and low enough not to significantly decrease intermolecular cross-linking of Hb molecules, due to a high ionic strength. Typically, about one mole of sulfhydryl functional groups (—SH) are needed to oxidation-stabilize between about 0.25 moles to about 5 moles of deoxy-Hb.

Optionally, prior to polymerizing the oxidation-stabilized deoxy-Hb, an appropriate amount of water is added to the polymerization reactor. In one embodiment, an appropriate amount of water is that amount which would result in a solution with a concentration of about 10 to about 100 g/l Hb when the oxidation-stabilized deoxy-Hb is added to the polymerization reactor. Preferably, the water is oxygen-depleted.

The temperature of the oxidation-stabilized deoxy-Hb solution in the polymerization reactor is raised to a temperature to optimize polymerization of the oxidation-stabilized deoxy-Hb when contacted with a cross-linking agent. Typically, the temperature of the oxidation-stabilized deoxy-Hb is about 25 to about 45° C., and in some embodiments, about 41 to about 43° C. throughout polymerization. An example of an acceptable heat transfer means for heating the polymerization reactor is a jacketed heating system which is heated by directing hot ethylene glycol through the jacket.

The oxidation-stabilized deoxy-Hb is then exposed to a suitable cross-linking agent at a temperature sufficient to polymerize the oxidation-stabilized deoxy-Hb to form a solution of polymerized hemoglobin poly(Hb)) over a period of about 2 hours to about 6 hours. A suitable amount of a cross-linking agent is that amount which will permit intramolecular cross-linking to stabilize the Hb and also intermolecular cross-linking to form polymers of Hb, to thereby increase intravascular retention. Typically, a suitable amount of a cross-linking agent is that amount wherein the molar ratio of cross-linking agent to Hb is in excess of about 2:1. Preferably, the molar ratio of cross-linking agent to Hb is between about 20:1 to 40:1.

Examples of suitable cross-linking agents include polyfunctional agents that will cross-link Hb proteins, such as glutaraldehyde, succindialdehyde, activated forms of polyoxyethylene and dextran, α-hydroxy aldehydes, such as glycolaldehyde, N-maleimido-6-aminocaproyl-(2′-nitro, 4′-sulfonic acid)-phenyl ester, m-maleimidobenzoic acid-N-hydroxysuccinimide ester, succinimidyl 4-(N-maleimidomethyl)cyclohexane-1-carboxylate, sulfosuccinimidyl 4-(N-maleimidomethyl)cyclohexane-1-carboxylate, m-maleimidobenzoyl-N-hydroxysuccinimide ester, m-maleimidobenzoyl-N-hydroxysulfosuccinimide ester, N-succinimidyl(4-iodoacetyl)aminobenzoate, sulfosuccinimidyl(4-iodoacetyl)aminobenzoate, succinimidyl 4-(p-maleimidophenyl)butyrate, sulfosuccinimidyl 4-(p-maleimidophenyl)butyrate, 1-ethyl-3-(3-dimethylaminopropyl)carbodiimide hydrochloride, N,N′-phenylene dimaleimide, and compounds belonging to the bis-imidate class, the acyl diazide class or the aryl dihalide class, among others.

Poly(Hb) is defined as having significant intramolecular cross-linking if a substantial portion (e.g., at least about 50%) of the Hb molecules are chemically bound in the poly(Hb), and only a small amount, such as less than about 15%, are contained within high molecular weight poly(Hb) chains. High molecular weight poly(Hb) molecules have a molecule weight, for example, above about 500,000 Daltons.

In a preferred embodiment, glutaraldehyde is used as the cross-linking agent. Typically, about 10 to about 70 grams of glutaraldehyde are used per kilogram of oxidation-stabilized deoxy-Hb. More preferably, glutaraldehyde is added over a period of five hours until approximately 29-31 grams of glutaraldehyde are added for each kilogram of oxidation-stabilized deoxy-Hb.

Wherein the cross-linking agent used is not an aldehyde, the poly(Hb) formed is generally a stable poly(Hb). Wherein the cross-linking agent used is an aldehyde, the poly(Hb) formed is generally not stable until mixed with a suitable reducing agent to reduce less stable bonds in the poly(Hb) to form more stable bonds. Examples of suitable reducing agents include sodium borohydride, sodium cyanoborohydride, sodium dithionite, trimethylamine, t-butylamine, morpholine borane and pyridine borane. The poly(Hb) solution is optionally concentrated by ultrafiltration until the concentration of the poly(Hb) solution is increased to between about 75 and about 85 g/l. For example, a suitable ultrafilter is a 30,000 Dalton filter (e.g., Millipore Helicon Cat #CDUF050LT; Amicon Cat #540430).

The pH of the poly(Hb) solution is then adjusted to the alkaline pH range to preserve the reducing agent and to prevent hydrogen gas formation, which can denature Hb during the subsequent reduction. The poly(Hb) is typically purified to remove non-polymerized hemoglobin. This can be accomplished by dialfiltration or hydroxyapatite chromatography (see, e.g. U.S. Pat. No. 5,691,453, which is incorporated herein by reference in its entirety). Following pH adjustment, at least one reducing agent, preferably a sodium borohydride solution, is added to the polymerization step typically through the deoxygenation loop. The pH and electrolytes of the stable poly(Hb) can then be restored to physiologic levels to form a stable polymerized hemoglobin-based oxygen carrier, by diafiltering the stable poly(Hb) with a diafiltration solution having a suitable pH and physiologic electrolyte levels.

Suitable methods of cross-linking hemoglobin and preserving the hemoglobin-based oxygen carrier are discussed in detail in U.S. Pat. No. 5,691,452, issued Nov. 25, 1997, which is incorporated herein by reference in its entirety.

### Filtration

The disclosed methods and systems comprise steps for the filtration, diafiltration, ultrafiltration, and straining of various intermediate hemoglobin solutions. Diafiltration is a dilution process that involves removal or separation of components (permeable molecules like salts, small proteins, solvents etc.,) of a solution based on their molecular size by using micro-molecule permeable filters in order to obtain a pure solution. Ultrafiltration (UF) is a membrane filtration process similar to Reverse Osmosis, using hydrostatic pressure to force water through a semi-permeable membrane. Filters and membranes may vary in their characteristics, e.g., molecular weight cutoff (MWCO), depending on the stage of the process within which the solution is being filtered. Filtration may also be used as a means for (or in tandem with) buffer exchange and/or concentration.

### Characteristics of Stabilized Hemoglobin Solutions

Stabilized hemoglobin solutions according to the present disclosure may have one or more characteristics that make them particularly suitable for in vitro, in vivo, experimental, and/or therapeutic applications. In some embodiments, the stabilized hemoglobin solutions may have one or more of the following attributes: high hemoglobin concentration, low dissolved oxygen concentration, low endotoxin concentration, long half-life, high average molecular weight, and a high percentage of greater-than-dimeric polymers of hemoglobin.

In some embodiments, a stabilized hemoglobin solution according to the present disclosure may have a higher concentration than other hemoglobin-based oxygen carriers or hemoglobin-based blood substitutes that are commercially available or under clinical review. In some embodiments, a stabilized hemoglobin solution of the present disclosure may have a concentration of about 150 g/L to about 200 g/L. In some embodiments, a stabilized hemoglobin solution of the present disclosure may have a concentration of at least about 150 g/L. In some embodiments, a stabilized hemoglobin solution of the present disclosure may have a concentration of at most about 200 g/L. In some embodiments, a stabilized hemoglobin solution of the present disclosure may have a concentration of about 150 g/L to about 155 g/L, about 150 g/L to about 160 g/L, about 150 g/L to about 165 g/L, about 150 g/L to about 170 g/L, about 150 g/L to about 175 g/L, about 150 g/L to about 180 g/L, about 150 g/L to about 185 g/L, about 150 g/L to about 190 g/L, about 150 g/L to about 195 g/L, about 150 g/L to about 200 g/L, about 155 g/L to about 160 g/L, about 155 g/L to about 165 g/L, about 155 g/L to about 170 g/L, about 155 g/L to about 175 g/L, about 155 g/L to about 180 g/L, about 155 g/L to about 185 g/L, about 155 g/L to about 190 g/L, about 155 g/L to about 195 g/L, about 155 g/L to about 200 g/L, about 160 g/L to about 165 g/L, about 160 g/L to about 170 g/L, about 160 g/L to about 175 g/L, about 160 g/L to about 180 g/L, about 160 g/L to about 185 g/L, about 160 g/L to about 190 g/L, about 160 g/L to about 195 g/L, about 160 g/L to about 200 g/L, about 165 g/L to about 170 g/L, about 165 g/L to about 175 g/L, about 165 g/L to about 180 g/L, about 165 g/L to about 185 g/L, about 165 g/L to about 190 g/L, about 165 g/L to about 195 g/L, about 165 g/L to about 200 g/L, about 170 g/L to about 175 g/L, about 170 g/L to about 180 g/L, about 170 g/L to about 185 g/L, about 170 g/L to about 190 g/L, about 170 g/L to about 195 g/L, about 170 g/L to about 200 g/L, about 175 g/L to about 180 g/L, about 175 g/L to about 185 g/L, about 175 g/L to about 190 g/L, about 175 g/L to about 195 g/L, about 175 g/L to about 200 g/L, about 180 g/L to about 185 g/L, about 180 g/L to about 190 g/L, about 180 g/L to about 195 g/L, about 180 g/L to about 200 g/L, about 185 g/L to about 190 g/L, about 185 g/L to about 195 g/L, about 185 g/L to about 200 g/L, about 190 g/L to about 195 g/L, about 190 g/L to about 200 g/L, or about 195 g/L to about 200 g/L. In some embodiments, a stabilized hemoglobin solution of the present disclosure may have a concentration of about 150 g/L, about 155 g/L, about 160 g/L, about 165 g/L, about 170 g/L, about 175 g/L, about 180 g/L, about 185 g/L, about 190 g/L, about 195 g/L, or about 200 g/L.

In some embodiments, a stabilized hemoglobin solution of the present disclosure may have a lower oxygen concentration than other hemoglobin-based oxygen carriers or hemoglobin-based blood substitutes that are commercially available or under clinical review. In some embodiments, the dissolved oxygen concentration is less than 0.1 mg/mL, less than 0.09 mg/mL, less than 0.08 mg/mL, less than 0.07 mg/mL, less than 0.06 mg/mL, less than 0.05 mg/mL, less than 0.04 mg/mL, less than 0.03 mg/mL, less than 0.02 mg/mL, or less than 0.01 mg/mL. In some embodiments, the dissolved oxygen concentration is less than 0.02 mg/mL. In some embodiments, the stabilized hemoglobin solution comprises less than 5% oxygenated hemoglobin as a percentage of overall hemoglobin. In some embodiments, the stabilized hemoglobin solution comprises less than 10%, less than 9%, less than 8%, less than 7%, less than 6%, less than 5%, less than 4%, less than 3%, or less than 2% oxygenated hemoglobin as a percentage of overall hemoglobin. In some embodiments, the stabilized hemoglobin solution comprises less than 3% oxygenated hemoglobin as a percentage of overall hemoglobin.

In some embodiments, the stabilized hemoglobin solution may contain little to no endotoxin contamination. In some embodiments, the stabilized hemoglobin solution is substantially free of endotoxins, phospholipids and non-hemoglobin proteins such as enzymes. In some embodiments, the stabilized hemoglobin solution may be virtually free of endotoxins. In some embodiments, the endotoxin concentration of a stabilized hemoglobin solution according to the present disclosure may be less than about 0.05 endotoxin units (EU) per milliliter (mL). In some embodiments, the endotoxin concentration of a stabilized hemoglobin solution according to the present disclosure may be less than about 0.5, 0.4, 0.3, 0.2, 0.1, 0.05, 0.04, 0.03, 0.02, or 0.01 EU per mL. In some embodiments, the measured endotoxins may comprise one or more of a cellular lipid, a cellular lipid layer and a lipopolysaccharide. In some embodiments, the endotoxin may be derived or isolated from a human cell. In some embodiments, the endotoxin may be derived or isolated from a non-human vertebrate cell. In some embodiments, the endotoxin may be derived or isolated from a microbe. In some embodiments, the endotoxin may be derived or isolated from a bacterium. In some embodiments, the endotoxin may be derived or isolated from a virus.

In some embodiments, the stabilized hemoglobin solution may comprise a distribution of hemoglobin oligomers of different sizes. In some embodiments, the stabilized hemoglobin solution may comprise virtually no hemoglobin monomers. In some embodiments, the stabilized hemoglobin solution may comprise less than 15%, less than 10%, less than 9%, less than 8%, less than 7%, less than 6%, or less than 5% hemoglobin dimers. In some embodiments, the stabilized hemoglobin solution may comprise less than 5% hemoglobin dimers. In some embodiments, the stabilized hemoglobin solution may comprise greater than 80%, greater than 85%, or greater than 90% hemoglobin oligomers between 68,000 daltons and 500,000 daltons. In some embodiments, the stabilized hemoglobin solution may comprise between 20% to 35% hemoglobin tetramers. In some embodiments, the stabilized hemoglobin solution may comprise about 20%, about 21%, about 22%, about 23%, about 24%, about 25%, about 26%, about 27%, about 28%, about 29%, about 30%, about 31%, about 32%, about 33%, about 34%, or about 35% hemoglobin tetramers. In some embodiments, the stabilized hemoglobin solution may comprise about 25% hemoglobin tetramers. In some embodiments, the hemoglobin solution may comprise between 15% and 25% hemoglobin octamers. In some embodiments, the stabilized hemoglobin solution may comprise about 15%, about 16%, about 17%, about 18%, about 19%, about 20%, about 21%, about 22%, about 23%, about 24%, or about 25% hemoglobin octamers. In some embodiments, the stabilized hemoglobin solution may comprise about 20% hemoglobin octamers. In some embodiments, the stabilized hemoglobin solution may comprise between 40% and 55% hemoglobin oligomers of greater-than-octamer size. In some embodiments, the stabilized hemoglobin solution may comprise about 40%, about 41%, about 42%, about 43%, about 44%, about 45%, about 46%, about 47%, about 48%, about 49%, about 50%, about 51%, about 52%, about 53%, about 54%, or about 55% hemoglobin oligomers of greater-than-octamer molecular weight. In some embodiments, the stabilized hemoglobin solution comprises about 50% hemoglobin oligomers of greater-than-octamer molecular weight.

In some embodiments, the stabilized hemoglobin solution comprises hemoglobin oligomers with a defined molecular weight distribution of greater than about 90% between 68,000 daltons and 500,000 daltons. In some embodiments, the stabilized hemoglobin solution may comprise hemoglobin oligomers having an average molecular weight of 200 kilodaltons (kDa).

The existence of methemoglobin may reduce the ability of the hemoglobin solution to release oxygen. In some embodiments, the stabilized hemoglobin solution comprises less than 10% methemoglobin as a percentage of overall hemoglobin. In some embodiments, the stabilized hemoglobin solution comprises less than 10%, less than 9%, less than 8%, less than 7%, less than 6%, less than 5%, less than 4%, less than 3%, less than 2%, less than 1%, or less than 0.5% methemoglobin as a percentage of overall hemoglobin. In some embodiments, the stabilized hemoglobin solution comprises less than about 1% methemoglobin as a percentage of overall hemoglobin.

In some embodiments, the stabilized hemoglobin has a longer half-life than non-stabilized or oxygenated hemoglobin and minimizes breakdown of tetrameric hemoglobin into dimers that cause renal toxicity. In some embodiments, the stabilized hemoglobin has a half life of at least 60 minutes, at least 90 minutes, at least 120 minutes, at least 150 minutes, at least 180 minutes, at least 210 minutes, or at least 240 minutes. In some embodiments, the stabilized hemoglobin has a half life of about 3.5 hours or about 210 minutes.

### Hemoglobin Sequences

Nucleic acid molecules useful in the methods of the invention include any nucleic acid molecule that encodes a polypeptide of heme iron composition of the invention or a fragment thereof. The encoded polypeptides need not be 100% identical with the polypeptides encoded by an endogenous nucleic acid sequence, but may exhibit substantial identity, e.g., at least 80%, at least 85%, at least 90%, at least 95%, or at least 99% identity.

In some embodiments, the hemoglobin comprised by the present stabilized hemoglobin solutions comprises a subunit alpha (a), wherein the subunit α comprises the amino acid sequence of:

(SEQ ID NO: Y)

| | |
|---|---|
| 1 | MVLSPADKTN VKAAWGKVGA HAGEYGAEAL ERMFLSFPTT |
| | KTYFPHFDLS HGSAQVKGHG |
| 61 | KKVADALTNA VAHVDDMPNA LSALSDLHAH KLRVDPVNFK |
| | LLSHCLLVTL AAHLPAEFTP |
| 121 | AVHASLDKFL ASVSTVLTSK YR. |

In some embodiments, the hemoglobin comprises a subunit α comprising an amino acid sequence having at least 50%, at least 60%, at least 70%, at least 80%, at least 90%, at least 95%, at least 98%, or at least 99% identity to the sequence of SEQ ID NO: Y. In some embodiments, the hemoglobin comprises a subunit α comprising an amino acid sequence having at least 90% identity to the sequence of SEQ ID NO: Y.

In some embodiments, the hemoglobin comprises a subunit α, wherein the subunit α is encoded by the nucleic acid sequence of:

(SEQ ID NO: Z)

| | |
|---|---|
| 1 | actcttctgg tccccacaga ctcagagaga acccaccatg |
| | gtgctgtctc ctgccgacaa |
| 61 | gaccaacgtc aaggccgcct ggggcaaggt tggcgcgcac |
| | gctggcgagt atggtgcgga |
| 121 | ggccctggag aggatgttcc tgtccttccc caccaccaag |
| | acctacttcc cgcacttcga |
| 181 | cctgagccac ggctctgccc aggttaaggg ccacggcaag |
| | aaggtggccg acgcgctgac |
| 241 | caacgccgtg gcgcacgtgg acgacatgcc caacgcgctg |
| | tccgccctga gcgacctgca |
| 301 | cgcgcacaag cttcgggtgg acccggtcaa cttcaagctc |
| | ctaagccact gcctgctggt |
| 361 | gaccctggcc gcccacctcc ccgccgagtt cacccctgcg |
| | gtgcacgcct ccctggacaa |
| 421 | gttcctggct tctgtgagca ccgtgctgac ctccaaatac |
| | cgttaagctg gagcctcggt |
| 481 | agcagttcct cctgccagat gggcctccca acgggccctc |
| | ctcccctcct tgcaccggcc |
| 541 | cttcctggtc tttgaataaa gtctgagtgg gcggc. |

In some embodiments, the hemoglobin comprises a subunit α, wherein the subunit α is encoded by a nucleic acid sequence having at least 50%, at least 60%, at least 70%, at least 80%, at least 90%, at least 95%, at least 98%, or at least 99% identity to the sequence of SEQ ID NO: Z. In some embodiments, the hemoglobin comprises a subunit α, wherein the subunit α is encoded by a nucleic acid sequence having at least 90% identity to the sequence of SEQ ID NO: Z.

In some embodiments, the hemoglobin comprises a subunit beta (β), wherein the subunit β comprises the amino acid sequence of:

(SEQ ID NO: Y1)

| | |
|---|---|
| 1 | MVHLTPEEKS AVTALWGKVN VDEVGGEALG RLLVVYPWTQ |
| | RFFESFGDLS TPDAVMGNPK |
| 61 | VKAHGKKVLG AFSDGLAHLD NLKGTFATLS ELHCDKLHVD |
| | PENFRLLGNV LVCVLAHHFG |
| 121 | KEFTPPVQAA YQKVVAGVAN ALAHKYH. |

In some embodiments, the hemoglobin comprises a subunit β, wherein the subunit β comprises an amino acid sequence having at least 50%, at least 60%, at least 70%, at least 80%, at least 90%, at least 95%, at least 98%, or at least 99% identity to the sequence of SEQ ID NO: Y1. In some embodiments, the hemoglobin comprises a subunit β, wherein the subunit β comprises an amino acid sequence having at least 90% identity to the sequence of SEQ ID NO: Y1.

In some embodiments, the hemoglobin comprises a subunit β, wherein the subunit β is encoded by the nucleic acid sequence of:

(SEQ ID NO: Z1)

| | |
|---|---|
| 1 | acatttgctt ctgacacaac tgtgttcact agcaacctca |
| | aacagacacc atggtgcatc |
| 61 | tgactcctga ggagaagtct gccgttactg ccctgtgggg |
| | caaggtgaac gtggatgaag |
| 121 | ttggtggtga ggccctgggc aggctgctgg tggtctaccc |
| | ttggacccag aggttctttg |
| 181 | agtcctttgg ggatctgtcc actcctgatg ctgttatggg |
| | caaccctaag gtgaaggctc |
| 241 | atggcaagaa agtgctcggt gcctttagtg atggcctggc |
| | tcacctggac aacctcaagg |
| 301 | gcacctttgc cacactgagt gagctgcact gtgacaagct |
| | gcacgtggat cctgagaact |
| 361 | tcaggctcct gggcaacgtg ctggtctgtg tgctggccca |
| | tcactttggc aaagaattca |
| 421 | ccccaccagt gcaggctgcc tatcagaaag tggtggctgg |
| | tgtggctaat gccctggccc |
| 481 | acaagtatca ctaagctcgc tttcttgctg tccaatttct |
| | attaaaggtt cctttgttcc |
| 541 | ctaagtccaa ctactaaact gggggatatt atgaagggcc |
| | ttgagcatct ggattctgcc |
| 601 | taataaaaaa catttatttt cattgcaa. |

In some embodiments, the hemoglobin comprises a subunit β, wherein the subunit β is encoded by a nucleic acid sequence having at least 50%, at least 60%, at least 70%, at least 80%, at least 90%, at least 95%, at least 98%, or at least 99% identity to the sequence of SEQ ID NO: Z1. In some embodiments, the hemoglobin comprises a subunit β, wherein the subunit β is encoded by a nucleic acid sequence having at least 90% identity to the sequence of SEQ ID NO: Z1.

In some embodiments, the hemoglobin comprises a subunit gamma (γ), wherein the subunit γ comprises the amino acid sequence of:

(SEQ ID NO: YF1)

| | |
|---|---|
| 1 | MGHFTEEDKA TITSLWGKVN VEDAGGETLG RLLVVYPWTQ |
| | RFFDSFGNLS SASAIMGNPK |
| 61 | VKAHGKKVLT SLGDAIKHLD DLKGTFAQLS ELHCDKLHVD |
| | PENFKLLGNV LVTVLAIHFG |
| 121 | KEFTPEVQAS WQKMVTGVAS ALSSRYH, |

In some embodiments, the hemoglobin comprises a subunit γ, wherein the subunit γ comprises an amino acid sequence having at least 50%, at least 60%, at least 70%, at least 80%, at least 90%, at least 95%, at least 98%, or at least 99% identity to the sequence of SEQ ID NO: YF1. In some embodiments, the hemoglobin comprises a subunit γ, wherein the subunit γ comprises an amino acid sequence having at least 90% identity to the sequence of SEQ ID NO: YF1.

In some embodiments, the hemoglobin comprises a subunit γ, wherein the subunit γ is encoded by the nucleic acid sequence of:

(SEQ ID NO: ZF1)

| | |
|---|---|
| 1 | acactcgctt ctggaacgtc tgaggttatc aataagctcc |
| | tagtccagac gccatgggtc |
| 61 | atttcacaga ggaggacaag gctactatca caagcctgtg |
| | gggcaaggtg aatgtggaag |
| 121 | atgctggagg agaaaccctg ggaaggctcc tggttgtcta |
| | cccatggacc cagaggttct |
| 181 | ttgacagctt tggcaacctg tcctctgcct ctgccatcat |
| | gggcaacccc aaagtcaagg |
| 241 | cacatggcaa gaaggtgctg acttccttgg gagatgccat |
| | aaagcacctg gatgatctca |
| 301 | agggcacctt tgcccagctg agtgaactgc actgtgacaa |
| | gctgcatgtg gatcctgaga |
| 361 | acttcaagct cctgggaaat gtgctggtga ccgttttggc |
| | aatccatttc ggcaaagaat |
| 421 | tcacccctga ggtgcaggct tcctggcaga agatggtgac |
| | tggagtggcc agtgccctgt |
| 481 | cctccagata ccactgagct cactgcccat gatgcagagc |
| | tttcaaggat aggctttatt |
| 541 | ctgcaagcaa tcaaataata aatctattct gctaagagat |
| | cacaca, |

In some embodiments, the hemoglobin comprises a subunit γ, wherein the subunit γ is encoded by a nucleic acid sequence having at least 50%, at least 60%, at least 70%, at least 80%, at least 90%, at least 95%, at least 98%, or at least 99% identity to the sequence of SEQ ID NO: ZF1. In some embodiments, the hemoglobin comprises a subunit γ, wherein the subunit γ is encoded by a nucleic acid sequence having at least 90% identity to the sequence of SEQ ID NO: ZF1.

In some embodiments, the hemoglobin comprises a subunit gamma (γ), wherein the subunit γ comprises the amino acid sequence of:

(SEQ ID NO: YF2)

| | |
|---|---|
| 1 | MGHFTEEDKA TITSLWGKVN VEDAGGETLG RLLVVYPWTQ |
| | RFFDSFGNLS SASAIMGNPK |
| 61 | VKAHGKKVLT SLGDATKHLD DLKGTFAQLS ELHCDKLHVD |
| | PENFKLLGNV LVTVLAIHFG |
| 121 | KEFTPEVQAS WQKMVTAVAS ALSSRYH, |

In some embodiments, the hemoglobin comprises a subunit γ, wherein the subunit γ comprises an amino acid sequence having at least 50%, at least 60%, at least 70%, at least 80%, at least 90%, at least 95%, at least 98%, or at least 99% identity to the sequence of SEQ ID NO: YF2. In some embodiments, the hemoglobin comprises a subunit γ, wherein the subunit γ comprises an amino acid sequence having at least 90% identity to the sequence of SEQ ID NO: YF2.

In some embodiments, the hemoglobin comprises a subunit γ, wherein the subunit γ is encoded by the nucleic acid sequence of:

(SEQ ID NO: ZF2)

| | |
|---|---|
| 1 | acactcgctt ctggaacgtc tgaggttatc aataagctcc |
| | tagtccagac gccatgggtc |
| 61 | atttcacaga ggaggacaag gctactatca caagcctgtg |
| | gggcaaggtg aatgtggaag |
| 121 | atgctggagg agaaaccctg ggaaggctcc tggttgtcta |
| | cccatggacc cagaggttct |
| 181 | ttgacagctt tggcaacctg tcctctgcct ctgccatcat |
| | gggcaacccc aaagtcaagg |
| 241 | cacatggcaa gaaggtgctg acttccttgg gagatgccac |
| | aaagcacctg gatgatctca |
| 301 | agggcacctt tgcccagctg agtgaactgc actgtgacaa |
| | gctgcatgtg gatcctgaga |
| 361 | acttcaagct cctgggaaat gtgctggtga ccgttttggc |
| | aatccatttc ggcaaagaat |
| 421 | tcacccctga ggtgcaggct tcctggcaga agatggtgac |
| | tgcagtggcc agtgccctgt |
| 481 | cctccagata ccactgagct cactgcccat gattcagagc |
| | tttcaaggat aggctttatt |
| 541 | ctgcaagcaa tacaaataat aaatctattc tgctgagaga |
| | tcac, |

In some embodiments, the hemoglobin comprises a subunit γ, wherein the subunit γ is encoded by a nucleic acid sequence having at least 50%, at least 60%, at least 70%, at least 80%, at least 90%, at least 95%, at least 98%, or at least 99% identity to the sequence of SEQ ID NO: ZF2. In some embodiments, the hemoglobin comprises a subunit γ, wherein the subunit γ is encoded by a nucleic acid sequence having at least 90% identity to the sequence of SEQ ID NO: ZF2.

### Systems for the Manufacture of Stabilized Hemoglobin Solutions

The present disclosure provides a system for the manufacture of stabilized hemoglobin solution. The system may be used to carry out a method according to any of the foregoing embodiments. In some embodiments, the system may comprise components as disclosed in the Examples and Figures. In some embodiments, the system makes use of numerous single use components in order to minimize the hemoglobin solution's exposure to endotoxins. Single use equipment may include tubing, bags, adaptors, filters, and the like. In some embodiments, the single use tubing results in a substantially endotoxin-free stabilized hemoglobin solution.

Generally, in some embodiments, the present disclosure provides a system for producing a stabilized hemoglobin composition comprising the means to carry out the following steps: 1) separation of red blood cells from the mammalian blood fraction; 2) hemolysis of the red blood cells to produce a composite of monomeric hemoglobin and stroma; 3) separation by filtration of the hemoglobin; 4) purification of the monomeric hemoglobin by high performance liquid chromatography (HPLC) to separate the hemoglobin from all other proteins residual of the red blood cells, as well as the phospholipid, enzyme and endotoxin contaminants; 5) deoxygenation and diafiltration; 6) cross-linking (polymerizing or aggregating) the monomeric hemoglobin; and/or 7) concentrating the stabilized hemoglobin solution.

In some embodiments, the system may comprise the means to carry out the steps of (1) unloading the blood raw product, (2) fractionating the blood raw product to produce a red blood cell fraction which is substantially free from white blood cells and platelets, β) osmotically disrupting the red blood cell fraction to produce a hemoglobin-containing solution, (4) clarifying the hemoglobin-containing solution to produce a hemoglobin solution which is substantially free of cellular debris, (5) microporously filtering the hemoglobin solution which is substantially free of cellular debris to produce a partially sterilized hemoglobin-containing solution, (6) ultrafiltering the partially sterilized hemoglobin-containing solution to produce a size-separated hemoglobin-containing solution, (7) chromatographically separating the size-separated hemoglobin-containing solution to produce a hemoglobin substantially free of phospholipids, non-hemoglobin proteins, and endotoxins, (8) deoxygenating the substantially endotoxin-free hemoglobin to produce a substantially deoxygenated hemoglobin solution, (9) cross-linking said substantially deoxygenated hemoglobin solution to produce stabilized hemoglobin solution, and/or (10) concentrating the stabilized hemoglobin solution, all steps done in a substantially endotoxin-free environment.

In some embodiments, the system may comprise means for carrying out a step after the cross-linking step to separate or partially separate monomeric and low molecular weight species of hemoglobin from the higher molecular weight polymers formed during cross-linking. In some embodiments, the system may comprise means for carrying out a step of concentrating the stabilized, deoxygenated hemoglobin solution to a concentration between 150 g/L and 200 g/L (inclusive of end points) of hemoglobin in solution.

In some embodiments, the system may comprise means for conducting any one or more of the above steps under conditions which result in a product which is substantially free of endotoxins, phospholipids and non-hemoglobin proteins such as enzymes, and has a defined molecular weight distribution of greater than about 90% between 68,000 daltons and 500,000 daltons.

Additional features of each of the steps are provided in the foregoing sections relating to the disclosed methods and may equally be implemented in the present inventive systems.

The following examples are intended to illustrate, but not to limit, embodiments of the disclosed methods and systems.

### EXAMPLES

### Example 1: Description of Manufacturing Process and Process Controls for Small Batch Stabilized Hemoglobin Solution Manufacture

### Blood Collection

Bovine blood is obtained from farms affiliated with the Universite de Montreal School of Veterinary Medicine. The animals are continuously observed through the school's documented health program.

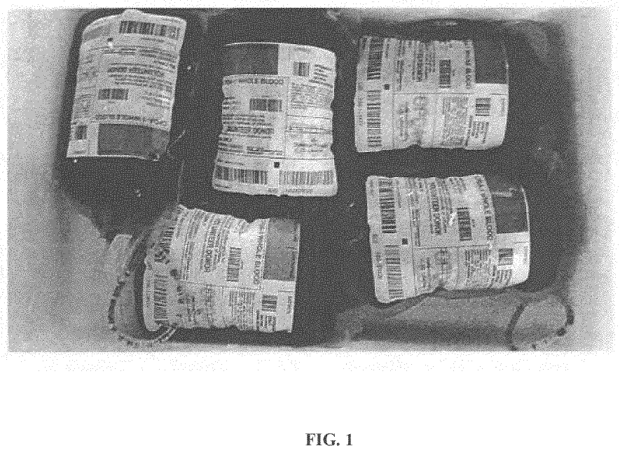

Blood in volumes of up to one (1) liter are obtained per animal via venipuncture from the coccygeal vein. Collection is made using a 500 milliliters (mL) Double Blood Pack collection system (Fenwal, part number 4R3429, Lake Zurich, Ill.). See FIG. 1. Bags contain CPD anticoagulant and are equipped with a satellite container and sterile needle/tubing sampling system. The cow's tail is raised and a 16 gauge needle is inserted about one-half inch deep and perpendicular to the tail and the underside, midline and three to six inches from the base of the tail. Blood is collected into the bag by gravity, until 450-500 mL are obtained. Immediately after collection, the bags are placed on ice and transported to the processing facility.

### Cell Washing

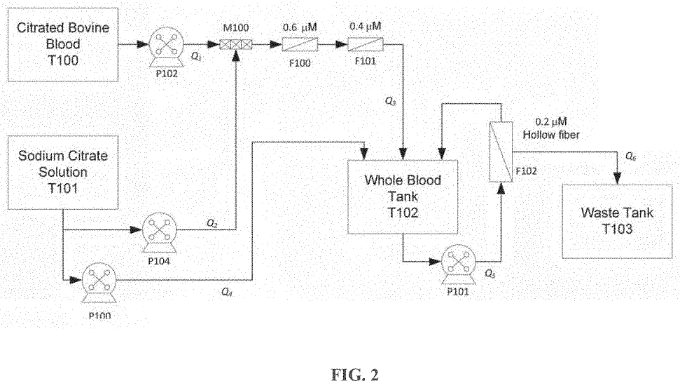

Collected blood is washed according to the process shown in FIG. 2. Blood, 3-5 liters (L), from multiple collections performed within the previous 24 hours, is transferred to a single Mobius 5 L flexible bag (T100) using a peristaltic pump. 50 L Sodium Citrate Solution (7.9 g/L sodium chloride and 6.0 g/L sodium citrate dihydrate with purified water) is prepared in a sterile mixing tank and depyrogenated by passage through a 10 kDa membrane filter into a 50 L flexible bag (T101). Citrated blood is pumped into a static in-line mixer at a flow rate of 200 mL-min-1, simultaneously with Sodium Citrate Solution at a flow rate of 280 mL-min-1. The mixture is directed through sequential 0.6 μM and 0.4 μM depth filtration membranes and into a 20 L flexible bag (T102). When bag T102 contains 5 L of filtered blood, the washing process is initiated by recirculation through a 0.2 μM hollow fiber membrane at a rate of 1 L-min-1. Transmembrane pressure is adjusted to 15 psi, allowing for an average permeate flow rate of 300 mL-min-1. Cell washing, by diafilitration, is initiated by pumping Sodium Citrate Solution into bag T102 at a flow rate of 300 mL-min-1, and continues until the cells are washed with 7 volumes. The diafiltration permeate is directed into a 50 L flexible waste bag (T103). Diafiltration continues until permeate equivalent to 7 blood volumes is collected. Examples of parts used for cell washing process is given in Table 1 below.

TABLE 1

| ID | Part | Manufacturer |
|---|---|---|
| T100 | Mobius 5 L | Merck Millipore |
| T101 | Mobius 50 L | Merck Millipore |
| T102 | Mobius 20 L | Merck Millipore |
| T103 | Mobius 50 L | Merck Millipore |
| P100 | Stainless Digital Process Pump | Masterflex |
| P101 | Stainless Digital Process Pump | Masterflex |
| F100 | | Sartorius |
| F101 | | |
| F102 | | |
| V100 | | |
| M100 | Static Mixer | Koflo |

An alternate to this process is to carry out this step using larger scale equipment or to install a centrifuge and carry out the c500 steps at 25 L. The disclosed set-up is designed to limit tank (bag size) to 50 L so that the bag can fit on a moveable rack.

### Cell Lysis

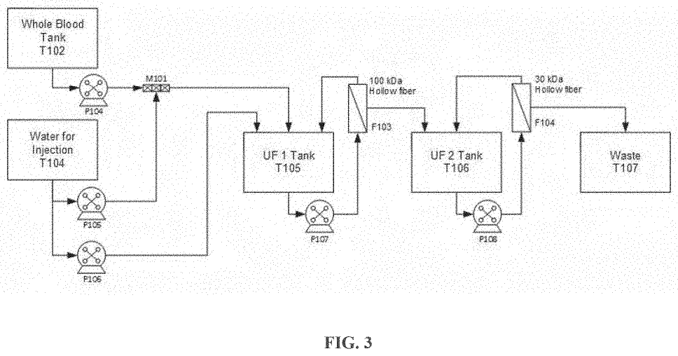

Hemoglobin is liberated from bovine red blood cells when cells are lysed by a rapid decrease in osmotic pressure. Cell lysis and sequential diafiltration across 100 kDa and 30 kDa membranes is carried out as shown in FIG. 3. Citrated Whole Blood is pumped into a static inline mixer at a flow rate of 250 mL-min-1, simultaneously with Water for Injection at a flow rate of 250 mL-min-1 into a 10 L flexible bag (T105). When T105 is filled with 2.0-2.5 L of diluted Whole Blood, recirculation is initiated through the 100,000 kDa hollow fiber membrane cartridge (F103) at a flow rate of 1000 mL-min-1. The permeate is directed to a 5 L flexible bag (T106). When 1.0-1.5 L of permeate has accumulated in T106, recirculation through the 30,000 kDa membrane (F104) is initiated at a flow rate of 1000 mL-min-1. The F104 permeate is directed to waste. Pumps 104 and 105 are stopped when the volume of Whole Blood (T102) is less than 250 mL. Diafiltration is then started by pumping WFI directly into T105 at a flow rate of for instance 250 mL-min-1 and continues until the hemoglobin concentration in the 100,000 kDa permeate is less than 0.2 mg-mL-1, corresponding to approximately 25-30 L diafiltration volume. Examples of parts used for cell lysis process is given in TABLE 2 below.

TABLE 2

| ID | Part | Manufacturer |
|---|---|---|
| T102 | Mobius 20 L | Merck Millipore |
| T104 | Mobius 50 L | Merck Millipore |
| T105 | Mobius 10 L | Merck Millipore |
| T106 | Mobius 5 L | Merck Millipore |
| T107 | Mobius 50 L | Merck Millipore |
| P104 | Stainless Digital Process Pump | Masterflex |
| P105 | Stainless Digital Process Pump | Masterflex |
| P106 | Stainless Digital Process Pump | Masterflex |
| P107 | Stainless Digital Process Pump | Masterflex |
| P108 | Stainless Digital Process Pump | Masterflex |
| F100 | | Sartorius |
| F101 | | |
| F102 | | |
| M101 | Static Mixer | Koflo |

### Deoxygenation of Hemoglobin Solution

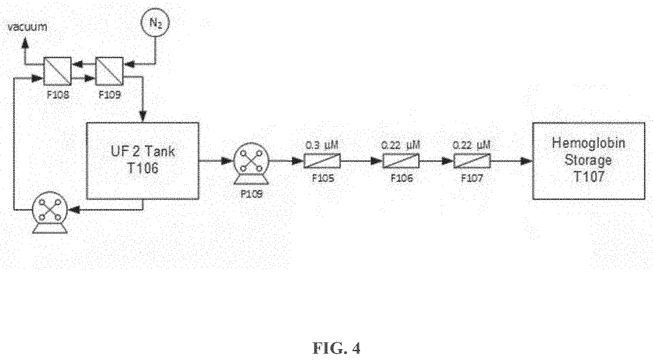

The hemoglobin solution is stabilized by removing oxygen and filtered for storage as an intermediate using a process depicted in FIG. 4. Initially, the hemoglobin solution is pumped through two Liquicell Membranes aligned in series at a flow rate of 500 ml-min-1, with a counter-current flow of nitrogen at 75 psi. Deoxygenation continues until the dissolved oxygen reading is below 0.02 mg-mL-1. When sufficient deoxygenation is achieved, the hemoglobin solution is filtered by pumping through a 0.3 μM and two 0.22 μM depth filters into a 5 L flexible bag. Filtered hemoglobin can be stored for up to 2 weeks before further processing. Examples of parts used for hemoglobin filtration-deoxygenation process is given in TABLE 3 below.

TABLE 3

| ID | Part | Manufacturer |
|---|---|---|
| T106 | Mobius 5 L | Merck Millipore |
| T107 | Mobius 5 L | Merck Millipore |
| P109 | Stainless Digital Process Pump | Masterflex |
| P110 | Stainless Digital Process Pump | Masterflex |
| F105 | 0.3 μM depth filter | Sartorius |
| F106 | 0.22 μM depth filter | |
| F107 | 0.22 μM depth filter | |
| F108 | Liquicel gas exchange membrane | 3M |
| F109 | Liquicel gas exchange membrane | 3M |

### Chromatography

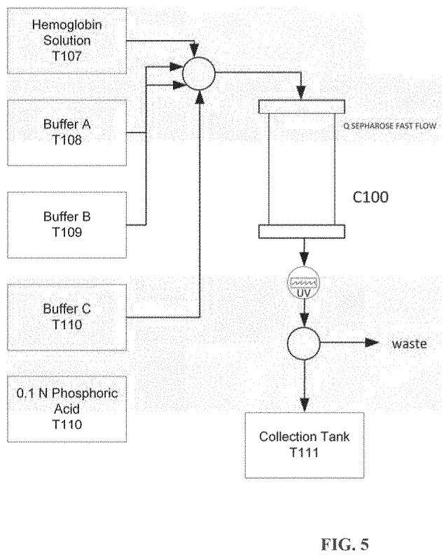

Chromatography is used to further purify the hemoglobin solution and reduce nonspecific blood cell components (process depicted in FIG. 5). This is performed using a GE Akta Biopilot chromatography system equipped with a GE Healthcare XK borosilicate column (5 cm i.d.×100 cm length) packed with Q Sepharose Fast Flow (GE Healthcare) to a bed height of 70±5 cm. Buffers are prepared using Water for Injection and filtered through a 10 kDa membrane to further reduce pyrogen content. Buffers are: (1) Buffer A; 2.42 g-L-1 tris base adjusted to pH 9.0±0.1 with acetic acid, (2) Buffer B; 6.05 g-L-1 Tris base adjusted to pH 7.0±0.1 with acetic acid and β) Buffer C; 2.42 g-L-1 Tris base and 58.38 g-L-1 NaCl adjusted to pH 8.9±0.1 with acetic acid.

Prior to the chromatographic operation, five complete buffer cycles are run through freshly packed Q Sepharose columns. Chromatography is carried out at a flow rate of 125 mL-min-1. Hemoglobin Solution, 1 L containing 130±10 mg-mL-1 hemoglobin, is initially loaded onto the column followed by the creation of a pH gradient formed by adding equal volumes of Buffer A and Buffer B. Protein eluting from the column is measured by UV absorbance at 280 nm. When absorbance of the eluate is falls below 0.05 AU, the column pH is increased by elution with 100% Buffer B. Hemoglobin elutes during this portion of the chromatographic run. The hemoglobin fraction is collected into a 20 L flexible bag (Ti 11) when the absorbance reaches 0.43 AU and terminates when the absorbance falls below 0.05 AU. Following elution of hemoglobin, 3 L of Buffer C is pumped through the column to elute tightly bound constituents.

The column is cleaned between each chromatographic run using 0.2 N phosphoric acid followed by two complete buffer cycles. Columns are stored in 0.2 N phosphoric acid if another run is not to be initiated within 24 hours. Examples of parts used for chromatography process is given in TABLE 4 below.

TABLE 4

| ID | Part | Manufacturer |
|---|---|---|
| T107 | Mobius 5 L | Merck Millipore |
| T108 | Mobius 50 L | Merck Millipore |
| T109 | Mobius 50 L | Merck Millipore |
| T110 | Mobius 50 L | Merck Millipore |
| T111 | Mobius 20 L | Merck Millipore |
| | Q Sepharose Fast Flow resin | GE |
| C100 | BioPilot chromatograpy System | GE |

### Deoxygenation

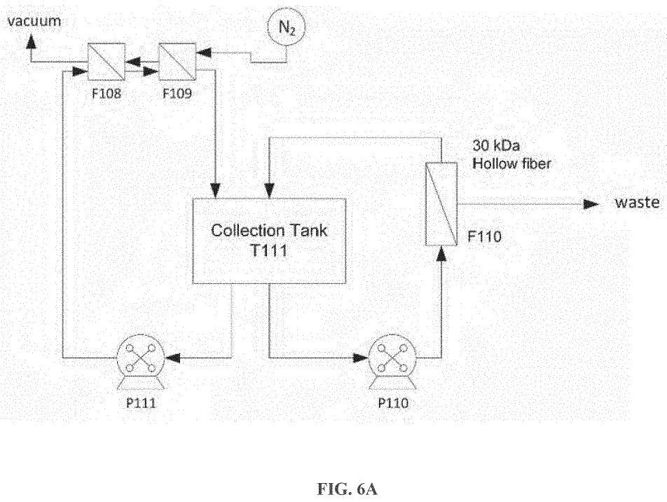

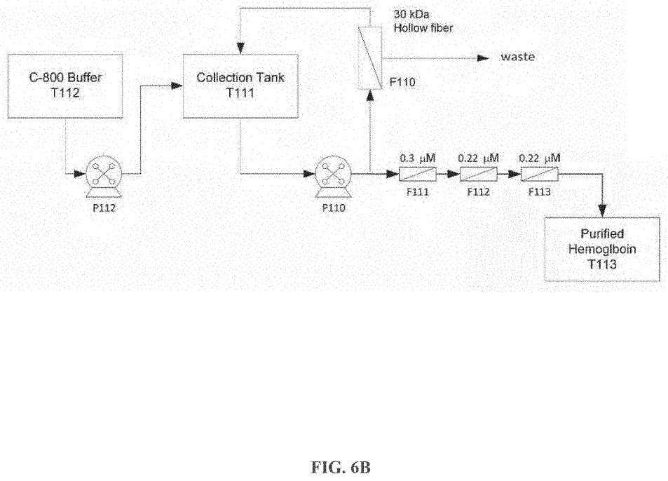

Purified Hemoglobin is deoxygenated to increase stability as shown in FIG. 6A-6B. Purified fractions from the anion exchange chromatography step are concentrated to 11.0±1 mg-mL-1 by filtration through a 30,000 Da hollow-fiber membrane (F1 10). When the desired hemoglobin concentration is reached, the Purified Hemoglobin is deoxygenated by passage through two Liquicell Membranes (F108, F109) aligned in series at a flow rate of 500 ml-min-1, with a counter-current flow of nitrogen at 75 psi. Deoxygenation continues until the dissolved oxygen reading is below 0.02 mg-mL-1.

The deoxygenated Purified Hemoglobin is subsequently diafiltered against six volumes of storage buffer by pumping through a 30,000 Da hollow-fiber membrane (F1 10). The composition of the storage buffer is 2.63 g-L-1 tribasic sodium phosphate dodecahydrate, 7.0 g-L-1 dibasic sodium phosphate heptahydrate and 2.0 g-L-1 acetylcysteine. When the buffer exchange is complete the solution is filtered by pumping through a 0.5 μM and two 0.22 μM depth filters into a 5 L flexible bag (Tl 13). The Purified Hemoglobin can be stored in a Nitrogen Glove Box for up to 60 days at room temperature (17-23° C.) before further processing. Examples of parts used for deoxygenation process is given in TABLE 5 below.

TABLE 5

| ID | Part | Manufacturer |
|---|---|---|
| T107 | Mobius 5 L | Merck Millipore |
| T108 | Mobius 50 L | Merck Millipore |
| T109 | Mobius 50 T | Merck Millipore |
| T110 | Mobius 50 L | Merck Millipore |
| T111 | Mobius 20 L | Merck Millipore |
| | Q Sepharose Fast Flow resin | GE |
| C100 | BioPilot chromatograpy System | GE |

### Polymerization

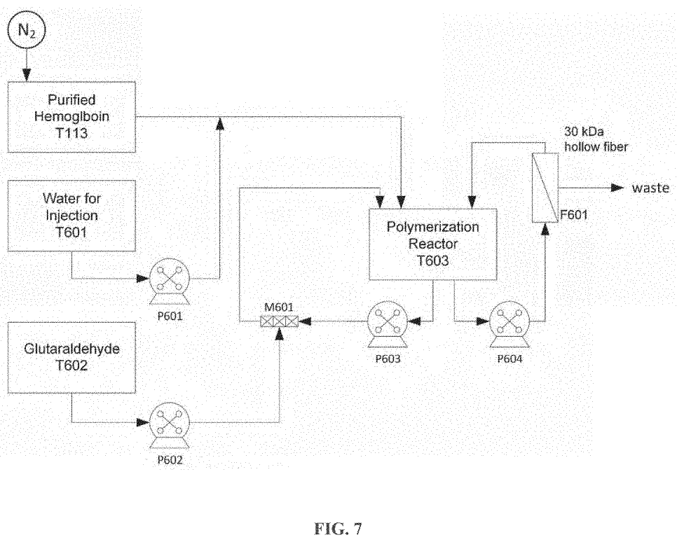

Purified Hemoglobin is polymerized by cross-linking with glutaraldehyde using the process depicted in FIG. 7. Purified Hemoglobin (4-5 L, 110 g/L) is transferred from Storage Tank (Tl 13) by under nitrogen pressure to a 20 L temperature controlled wave bag (T603). Water for Injection is pumped through the Purified Hemoglobin transfer line into T603 to reduce the hemoglobin concentration to 40 g/L. The temperature of the diluted Hemoglobin solution is then raised to 42±2° C. Glutaraldehyde solution is prepared at a concentration of 6.2 g/L in a temperature controlled Wave bag (T602) and heated to 42±2° C. The Glutaraldehyde solution is pumped into T603 at a rate of 10 mL/min until the ratio of glutaraldehyde to hemoglobin is approximately 0.029: 1. The glutaraldehyde is added through a static mixer (M601) in a recirculation loop to ensure rapid and homogeneous mixing with the hemoglobin solution. When the addition of glutaraldehyde is completed, the temperature of the reaction mixture is cooled to 22±2° C. and the solution is concentrated by diafiltration through a 30,000 Da hollow-fiber membrane (F601) to a hemoglobin concentration of 80±5 g/L.

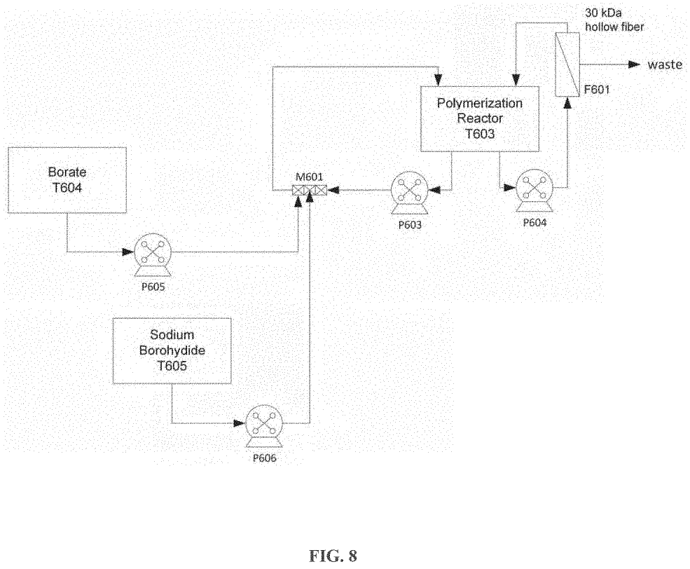

Glutaraldehyde-hemoglobin bonds are stabilized by reduction with sodium borohydride as summarized in FIG. 8. Sodium borohydride decomposes in aqueous solution at neutral pH to form molecular hydrogen and sodium borate. Diafiltration of polymerised hemoglobin with sodium borate buffer is carried out to stabilize sodium borohydride and limit hydrogen gas formation. Borate buffer is composed of 4.58 g/L sodium borate decahydrate and 0.91 g/L sodium hydroxide in Water for Injection.

The buffer is filtered through a 10,000 Da membrane to reduce pyrogen content and is stored in a 20 L flexible bag (T605). The borate buffer is pumped into T603, through the recirculation loop, initially at a flow rate of 250 mL/min. Simultaneously, the polymerized hemoglobin solution is diafiltered by pumping through a 30,000 Da hollow fiber membrane at a flow rate of 1,000 mL/min. The borate addition flow rate is adjusted to equal that of the diafiltration permeate rate, approximately 250 mL/min. Diafiltration with borate buffer continues until the volume corresponding to 3 times that of the polymerized hemoglobin solution have been added.

Sodium borohydride solution is comprised of 9.45 g/L sodium borohydride, 4.58 g/L sodium borate decahydrate and 0.91 g/L sodium hydroxide in Water for Injection. The solution is filtered through a 10,000 Da membrane to reduce pyrogen content and stored in a 2 L flexible bag (T606). Sodium Borohydride solution (0.6 L) is pumped into T603, through the recirculation loop, initially at a flow rate of 7 mL/min and the temperature of T603 controlled at 20±2° C. The borohydride reaction continues for 60 minutes after all the solution has been added, with continuous recirculation of the polymerized hemoglobin solution.

The stabilized polymerised hemoglobin solution is concentrated across the 30 kD ultrafiltration membrane (F601) to a hemoglobin concentration of 100±5 g/L. Boron containing components (sodium borate/sodium borohydride) are removed and the pH reduced to 8.0-8.4 by diafiltration of the polymerised hemoglobin across 30 kD ultrafiltration membrane (F601) with Diafiltration Solution A (6.67 g/L sodium chloride, 0.30 g/L potassium chloride, 0.20 g/L calcium chloride dihydrate, 0.445 g/L sodium hydroxide, 2.02 g/L N-acetyl-L-cysteine, 3.07 g/L sodium lactate, pH=4.9-5.1). Examples of parts used for the polymerization process is given in TABLE 6 below.

TABLE 6

| ID | Part | Manufacturer |
|---|---|---|
| T113 | Mobius 5 L | Merck Millipore |
| T601 | Mobius 50 L | Merck Millipore |
| T602 | Mobius 50 L | Merck Millipore |
| T603 | Mobius 50 L | Merck Millipore |
| T604 | Mobius 20 L | Merck Millipore |
| T605 | Q Sepharose Fast Flow resin | GE |
| P601 | Stainless Digital Process Pump | Masterflex |
| P602 | Stainless Digital Process Pump | Masterflex |
| P603 | Stainless Digital Process Pump | Masterflex |
| P604 | Stainless Digital Process Pump | Masterflex |
| P605 | Stainless Digital Process Pump | Masterflex |
| P606 | Stainless Digital Process Pump | Masterflex |
| M601 | Static Mixer | Kobi |

### Sterile Filtration

Final polymerised haemoglobin solution is filtered through a 0.5 μm depth filter, a sterilizing grade 0.2 μm membrane filter, and a 2nd sterilizing grade 0.2 μm membrane filter into a 275-liter steam sanitized portable bulk holding tank. The bulk holding tank is stored under nitrogen until use.

### Example 2: Description of Manufacture Process and Process Controls for Bulk Manufacturing of Stabilized Hemoglobin Solution

### Blood Collection

Bovine blood is obtained from farms affiliated with the Universite de Montreal School of Veterinary Medicine. The animals are continuously observed through the school's documented health program.

Blood in volumes of up to one (1) liter are obtained per animal via venipuncture from the coccygeal vein. Collection is made using a 500 mL Double Blood Pack collection system (Fenwal, part number 4R3429, Lake Zurich, Ill.). Bags contain CPD anticoagulant and are equipped with a satellite container and sterile needle/tubing sampling system. The cow's tail is raised and a 16 gauge needle is inserted about one-half inch deep and perpendicular to the tail and the underside, midline and three to six inches from the base of the tail. Blood is collected by into the bag by gravity, until 450-500 mL are obtained. Immediately after collection, the bags are placed on ice and transported to the processing facility.

### Cell Washing

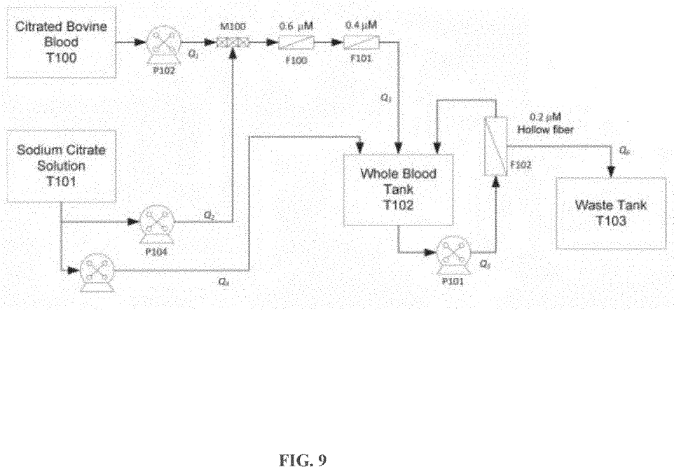

Collected blood is washed according the process shown FIG. 9. Blood, 15-20 L, from multiple collections performed within the previous 24 hours, is transferred to a single 20 L GE Ready Circuit flexible bag (T100) using a peristaltic pump. 200 L Sodium Citrate Solution (7.9 g/L sodium chloride and 6.0 g/L sodium citrate dihydrate with purified water) is prepared in a sterile mixing tank and depyrogenated by passage through a 10,000 Da membrane filter into a 200 L Ultra Low-Density Polyethylene (ULDP) single use bag (T101). Citrated blood is pumped into a static in-line mixer at a flow rate of 500 mL-min-1, simultaneously with Sodium Citrate Solution at a flow rate of 700 mL-min-1. The mixture is directed through sequential 0.6 μM and 0.4 μM depth filtration membranes and into a 50 L ULDP single use bag (T102). When bag T102 contains 10 L of filtered blood, the washing process is initiated by recirculation through a 0.2 μM hollow fiber membrane at a rate of 2 L-min-1. Transmembrane pressure is adjusted to 15 psi, allowing for an average permeate flow rate of 500 mL-min-1. Cell washing, by diafilitration, is initiated by pumping Sodium Citrate Solution into bag T102 at a flow rate of 500 mL-min-1, and continues until the cells are washed with 7 diafiltration volumes. The diafiltration permeate is directed into a 200 L ULDP single use bag (T103). Diafiltration continues until permeate equivalent to 7 blood volumes is collected.

Examples of parts used for cell wash process is given in TABLE 7 below, and examples of parts used for cell wash in-process testing is given in TABLE 8 below.

TABLE 7

| ID | Part | Manufacturer |
|---|---|---|
| T100 | Ready Circuit 20 L | GE Healthcare |
| T101 | Xcellerex XDM 200 L | GE Healthcare |
| T102 | Xcellerex XDM 50 L | GE Healthcare |
| T103 | Xcellerex XDM 200 L | GE Healthcare |
| P100 | Stainless Digital Process Pump | Masterflex |
| P101 | Stainless Digital Process Pump | Masterflex |
| F100 | 0.6 μM depth filter | Sartorius |
| F101 | 0.4 μM depth filter | Sarlorias |
| F102 | 0.2 μM hollow-fiber | Sartorius |
| M100 | Static Mixer | Koflo |

TABLE 8

| Test Material/Parameter | Measurement |
|---|---|
| Citrated Bovine Blood | Total Hemoglobin (Hgb) |
| Sodium Citrate Solution | LAL |
| F102 Permeate | Protein (UV280) |

### Cell Lysis

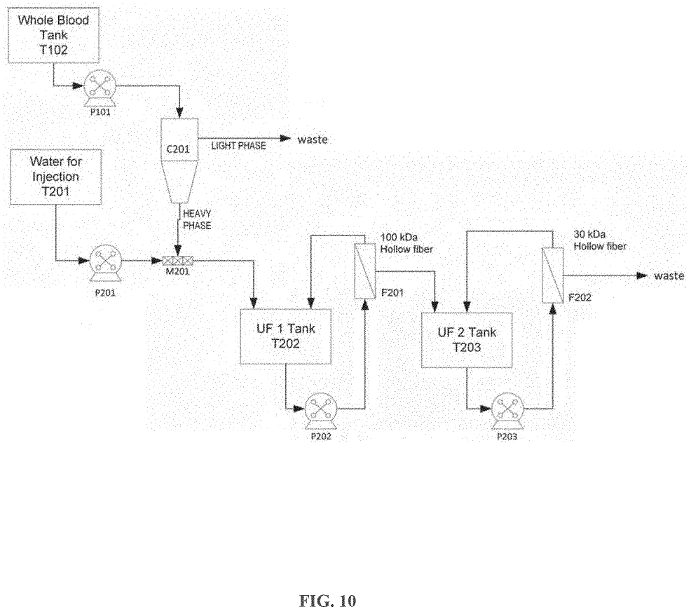

Red blood cells are separated from white blood cells and platelets by centrifugation and the hemoglobin liberated from red blood cells when cells are lysed by a rapid decrease in osmotic pressure as shown in FIG. 10. Washed blood cells are pumped into a tubular bowl centrifuge (C201) operating at 13,500×g. Red blood cells contained in the heavy phase are directed through a static mixer (M201), where they are diluted 2-fold with Water for Injection, and into a 20 L GE Ready Circuit flexible bag (T202). When T202 is filled with at least 10 L of diluted Whole Blood, recirculation is initiated through the 100,000 kDa hollow fiber membrane cartridge (F201) at a flow rate of 1000 mL-min-1. The permeate is directed to a 20 L GE Ready Circuit flexible bag (T203). When 15 L of permeate has accumulated in T203, recirculation through the 30,000 kDa membrane (F202) is initiated at a flow rate of 1000 mL-min-1. The F202 permeate is directed to waste. Diafiltration through the 100,000 Da membrane (F201) continues until the hemoglobin concentration in the permeate is less than 0.2 mg/mL, indicating that most of the liberated hemoglobin has been extracted. This corresponds to approximately 15-20 diafiltration volumes, corresponding to approximately 25-30 L diafiltration volume. Hemoglobin, separated from the cell debris by 100,000 Da filtration, is concentrated by filtration against a 30,000 kDa membrane. The 100,000 Da and 30,000 Da steps are carried out in a continuous process. The 30,000 Da filtration is stopped when the hemoglobin concentration is in the range of 90-1 10 g/L.

Examples of parts used for cell lysis process is given in TABLE 9 below, and examples of parts used for cell lysis in-process testing is given in TABLE 10 below.

TABLE 9

| ID | Part | Manufacturer |
|---|---|---|
| T102 | Xcellerex XDM 50 L | GE Healthcare |
| T201 | Xcellerex XDM 200 L | GE Healthcare |
| T202 | 20 L Ready Circuit | GE Healthcare |
| T203 | 20 L Ready Circuit | GE Healthcare |
| T204 | Xcellerex XDM 200 L | GE Healthcare |
| P101 | Stainless Digital Process Pump | Masterflex |
| P201 | Stainless Digital Process Pump | Masterflex |
| P202 | Stainless Digital Process Pump | Masterflex |
| P203 | Stainless Digital Process Pump | Masterflex |
| F201 | 100 kDa hollow-fiber | Sartorius |
| F202 | 30 kDa hollow-fiber | Sartorius |
| M201 | Static Mixer | Koflo |

TABLE 10

| Test Material/Parameter | Measurement |
|---|---|
| Citrated Bovine Blood | Total Hemoglobin (Hgb) |
| Water for Injection | LAL |
| 100,000 Da(F201) Permeate | Total Hemoglobin (Hgb) |
| 30,000 Da(F201) Retentate | Total Hemoglobin (Hgb) |

### Deoxygenation of Hemoglobin Solution

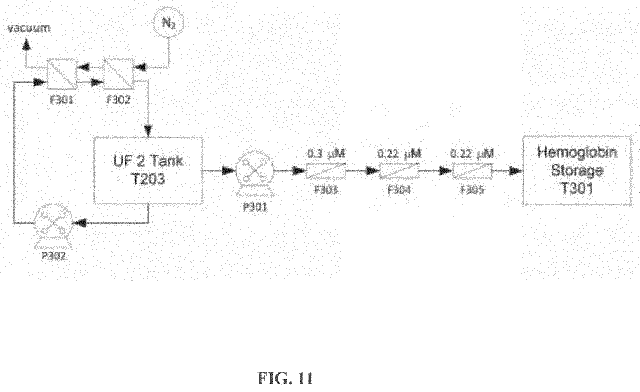

The hemoglobin solution is stabilized by removing oxygen and filtered for storage as an intermediate using a process depicted in FIG. 11. Initially, the hemoglobin solution is pumped through two Liquicell Membranes aligned in series at a flow rate of 500 ml-min-1, with a counter-current flow of nitrogen at 75 psi. Deoxygenation continues until the dissolved oxygen reading is below 0.02 mg/mL. When sufficient deoxygenation is achieved, the hemoglobin solution is filtered by pumping through a 0.3 μM and two 0.22 μM depth filters into a 20 L GE Ready Circuit flexible bag (T301). Filtered hemoglobin can be stored for up to 2 weeks before further processing.

Examples of parts used for hemoglobin filtration-deoxygenation process is given in TABLE 11 below, and examples of parts used for hemoglobin filtration-deoxygenation in-process testing is given in TABLE 12 below.

TABLE 11

| ID | Part | Manufacturer |
|---|---|---|
| T202 | 20 L Ready Circuit | GE Healthcare |
| T301 | 20 L Ready Circuit | GE Healthcare |
| P109 | Stainless Digital Process Pump | Masterflex |
| P110 | Stainless Digital Process Pump | Masterflex |
| F105 | 0.3 μM depth filter | Sartorius |
| F106 | 0.22 μM depth filter | Sartorius |
| F107 | 0.22 μM depth filter | Sartorius |
| F108 | Liquicel gas exchange membrane | 3M |
| F109 | Liquicel gas exchange membrane | 3M |

TABLE 12

| Test Material/Parameter | Measurement |
|---|---|
| Washed hemoglobin (T203) | Dissolved oxygen |
| | Total Hgb |
| | Met-Hgb |
| | Oxy-Hgb |
| Hemoglobin Storage | Dissolved oxygen |
| | Total Hgb |
| | Met-Hgb |
| | Oxy-Hgb |

### Chromatography

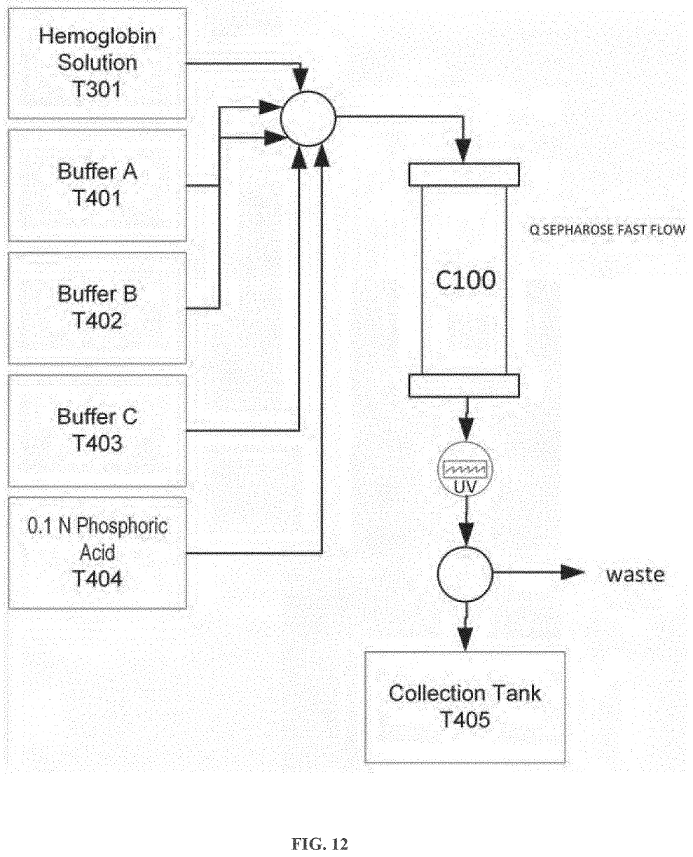

Chromatography is used to further purify the hemoglobin solution and reduce nonspecific blood cell components (process depicted in FIG. 12). This is performed using a GE Akta Biopilot chromatography system equipped with a GE Healthcare XK borosilicate column (5 cm i.d.×100 cm length) packed with Q Sepharose Fast Flow (GE Healthcare) to a bed height of 70±5 cm. Buffers are prepared using Water for Injection and filtered through a 10 kDa membrane to further reduce pyrogen content. Buffers are: (1) Buffer A; 2.42 g-L-1 tris base adjusted to pH 9.0±0.1 with acetic acid, (2) Buffer B; 6.05 g-L-1 Tris base adjusted to pH 7.0±0.1 with acetic acid and β) Buffer C; 2.42 g-L-1 Tris base and 58.38 g-L-1 NaCl adjusted to pH 8.9±0.1 with acetic acid.

Prior to the chromatographic operation, five complete buffer cycles are run through freshly packed Q Sepharose columns. Chromatography is carried out at a flow rate of 125 mL-min-1. Hemoglobin Solution, 1 L containing 130±10 mg-mL-1 hemoglobin, is initially loaded onto the column followed by the creation of a pH gradient formed by adding equal volumes of Buffer A and Buffer B. Protein eluting from the column is measured by UV absorbance at 280 nm. When absorbance of the eluate is falls below 0.05 AU, the column pH is increased by elution with 100% Buffer B. Hemoglobin elutes during this portion of the chromatographic run. The hemoglobin fraction is collected into a 20 L GE Ready Circuit single use bag (T405) when the absorbance reaches 0.43 AU and terminates when the absorbance falls below 0.05 AU. Following elution of hemoglobin, 3 L of Buffer C is pumped through the column to elute tightly bound constituents.

The column is cleaned between each chromatographic run using 0.2 N phosphoric acid followed by two complete buffer cycles. Columns are stored in 0.2 N phosphoric acid if another run is not to be initiated within 24 hours.

Examples of parts used for the chromatography process is given in TABLE 13 below, and examples of parts used for chromatography in-process testing is given in TABLE 14 below.

TABLE 13

| ID | Part | Manufacturer |
|---|---|---|
| T301 | 20 L Ready Circuit | GE Healthcare |
| T401 | 50 L Ready Circuit | GE Healthcare |
| T402 | 50 L Ready Circuit | GE Healthcare |
| T403 | 50 L Ready Circuit | GE Healthcare |
| T404 | 50 L Ready Circuit | GE Healthcare |
| | Q Sepharose Fast Flow resin | GE Healthcare |
| C100 | BioPilot chromatograpy System | GE Healthcare |

TABLE 14

| Test Material/Parameter | Measurement |
|---|---|
| Column eluate | UV280 |
| Chromatography Buffers | LAL |

### Deoxygenation

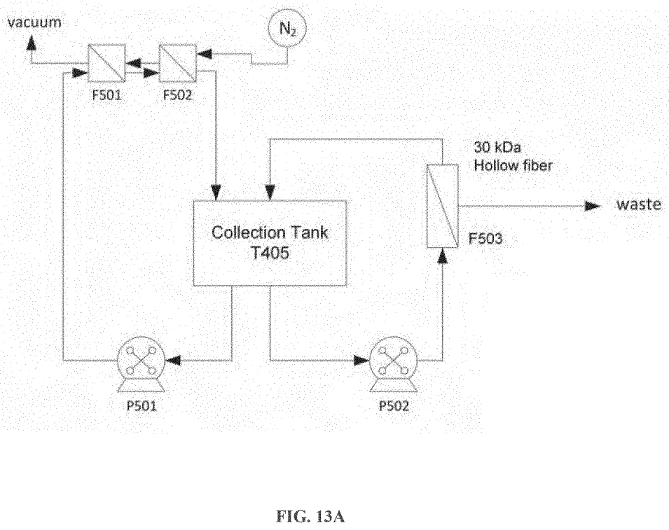

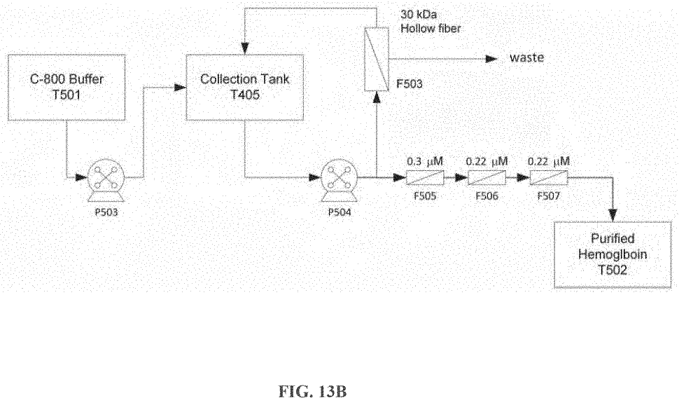

Purified Hemoglobin is deoxygenated to increase stability as shown in FIG. 13A and FIG. 13B. Purified fractions from the anion exchange chromatography step are concentrated to 11.0±1 mg-mL-1 by filtration through a 30,000 Da hollow-fiber membrane (F503). When the desired hemoglobin concentration is reached, the Purified Hemoglobin is deoxygenated by passage through two Liquicell Membranes (F501, F502) aligned in series at a flow rate of 500 ml-min-1, with a counter-current flow of nitrogen at 75 psi. Deoxygenation continues until the dissolved oxygen reading is below 0.02 mg/mL.

The deoxygenated Purified Hemoglobin is subsequently diafiltered against six volumes of storage buffer by pumping through a 30,000 Da hollow-fiber membrane (F1 10). The composition of the storage buffer is 2.63 g-L-1 tribasic sodium phosphate dodecahydrate, 7.0 g-L-ldibasic sodium phosphate heptahydrate and 2.0 g-L\hat{ }acetylcysteine. When the buffer exchange is completed the solution is filtered by pumping through a 0.5 μM and two 0.22 μM depth filters into a 20 L GE Ready Circuit single use bag (T501). The Purified Hemoglobin can be stored in a Nitrogen Glove Box for up to 60 days at room temperature (17-23° C.) before further processing.

Examples of parts used for the deoxygenation process is given in TABLE 15 below, and examples of parts used for deoxygenation in-process testing is given in TABLE 16 below.

TABLE 15

| ID | Part | Manufacturer |
|---|---|---|
| T405 | 20 L Ready Circuit | GE Healthcare |
| T501 | 50 L Ready Circuit | GE Healthcare |
| T502 | 20 L Ready Circuit | GE Healthcare |
| F501 | Liquicel gas exchange membrane | 3M |
| F502 | Liquicel gas exchange membrane | 3M |
| F503 | 30,000 Da hollow fiber | Sartorius |
| F504 | 0.3 uM depth filtration cartridge | Sartorius |
| F505 | 0.22 uM depth filtration cartridge | Sartorius |
| F506 | 0,22 uM depth filtration cartridge | Sartorius |

TABLE 16

| Test Material/Parameter | Measurement |
|---|---|
| Column eluate | UV280 |
| Chromatography Buffers | LAL |

### Polymerization

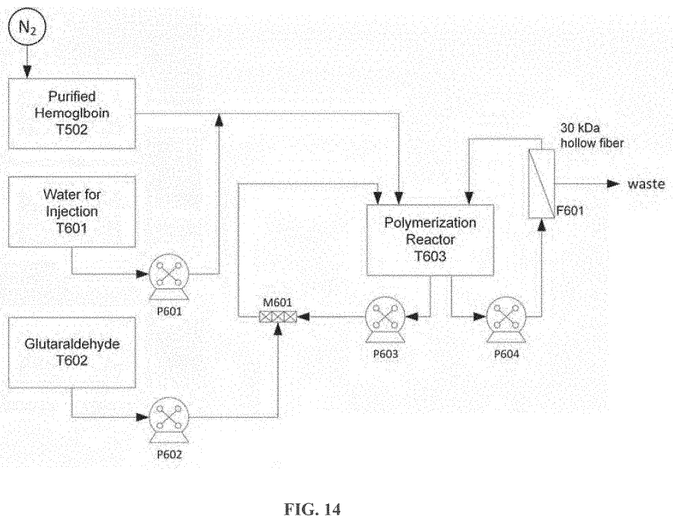

Purified Hemoglobin is polymerized by cross-linking with glutaraldehyde using the process depicted in FIG. 14. Purified Hemoglobin (4-5 L, 110 g/L) is transferred from Storage Tank (T501) by under nitrogen pressure to a 20 L temperature controlled Wave bag (T603). Water for Injection is pumped through the Purified Hemoglobin transfer line into T603 to reduce the hemoglobin concentration to 40 g/L. The temperature of the diluted Hemoglobin solution is then raised to 42±2° C. Glutaraldehyde solution is prepared at a concentration of 6.2 g/L in a temperature controlled Wave bag (T602) and heated to 42±2° C. The glutaraldehyde solution is pumped into T603 at a rate of 10 mL/min until the ratio of glutaraldehyde to hemoglobin is approximately 0.029: 1. The glutaraldehyde is added through a static mixer (M601) in a recirculation loop to ensure rapid and homogeneous mixing with the hemoglobin solution. When the addition of glutaraldehyde is completed, the temperature of the reaction mixture is cooled to 22±2° C. and the solution is concentrated by diafiltration through a 30,000 Da hollow-fiber membrane (F601) to a hemoglobin concentration of 80±5 g/L.

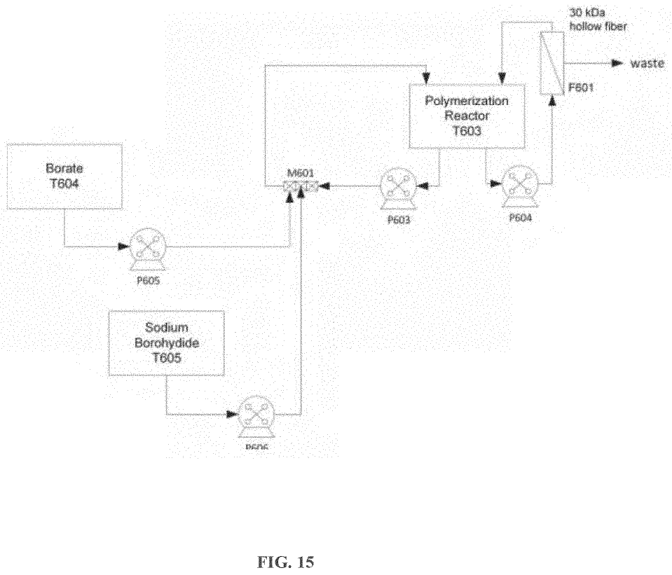

Glutaraldehyde-hemoglobin bonds are stabilized by reduction with sodium borohydride as summarized in FIG. 15. Sodium borohydride decomposes in aqueous solution at neutral pH to form molecular hydrogen and sodium borate. Diafiltration of polymerised hemoglobin with sodium borate buffer is carried out to stabilize sodium borohydride and limit hydrogen gas formation. Borate buffer is composed of 4.58 g/L sodium borate decahydrate and 0.91 g/L sodium hydroxide in Water for Injection. The buffer is filtered through a 10,000 Da membrane to reduce pyrogen content and is stored in a 20 L flexible bag (T605). The borate buffer is pumped into T603, through the recirculation loop, initially at a flow rate of 250 mL/min. Simultaneously, the polymerized hemoglobin solution is diafiltered by pumping through a 30,000 Da hollow fiber membrane at a flow rate of 1,000 mL/min. The borate addition flow rate is adjusted to equal that of the diafiltration permeate rate, approximately 250 mL/min. Diafiltration with borate buffer continues until the volume corresponding to 3 times that of the polymerized hemoglobin solution have been added.

Sodium borohydride solution is comprised of 9.45 g/L sodium borohydride, 4.58 g/L sodium borate decahydrate and 0.91 g/L sodium hydroxide in Water for Injection. The solution is filtered through a 10,000 Da membrane to reduce pyrogen content and stored in a 2 L flexible bag (T606). Sodium Borohydride solution (0.6 L) is pumped into T603, through the recirculation loop, initially at a flow rate of 7 mL/min and the temperature of T603 controlled at 20±2° C. The borohydride reaction continues for 60 minutes after all the solution has been added, with continuous recirculation of the polymerized hemoglobin solution.

The stabilized polymerised hemoglobin solution is concentrated across the 30 kDa ultrafiltration membrane (F601) to a hemoglobin concentration of 100±5 g/L. Boron containing components (sodium borate/sodium borohydride) are removed and the pH reduced to 8.0-8.4 by diafiltration of the polymerised hemoglobin across 30 kD ultrafiltration membrane (F601) with Diafiltration Solution A (6.67 g/L sodium chloride, 0.30 g/L potassium chloride, 0.20 g/L calcium chloride dihydrate, 0.445 g/L sodium hydroxide, 2.02 g/L N-acetyl-L-cysteine, 3.07 g/L sodium lactate, pH=4.9-5.1).

Examples of parts used for the polymerization process is given in TABLE 17 below, and examples of parts used for polymerization in-process testing is given in TABLE 18 below.

TABLE 17

| ID | Part | Manufacturer |
|---|---|---|
| T502 | 20 L Ready Circuit | GE Healthcare |
| T601 | 50 L Ready Circuit | GE Healthcare |
| T602 | 50 L Ready Circuit | GE Healthcare |
| T603 | 50 L Ready Circuit | GE Healthcare |
| T604 | 50 L Ready Circuit | GE Healthcare |
| T605 | 20 L Ready Circuit | GE Healthcare |
| P601 | Stainless Digital Process Pump | Masterflex |
| P602 | Stainless Digital Process Pump | Masterflex |
| P603 | Stainless Digital Process Pump | Masterflex |
| P604 | Stainless Digital Process Pump | Masterflex |
| P605 | Stainless Digital Process Pump | Masterflex |
| P606 | Stainless Digital Process Pump | Masterflex |
| M601 | Static Mixer | Kobi |
| F601 | 30,000 Da Hollow Fiber | Sartorius |

TABLE 18

| Test Material/Parameter | Measurement |
|---|---|
| Column eluate | UV280 |
| Chromatography Buffers | LAL |

### Sterile Filtration

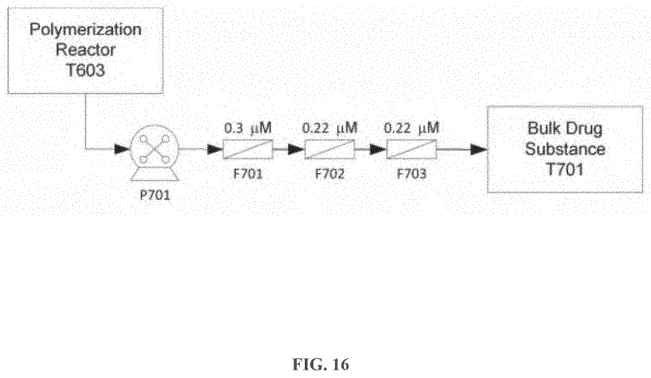

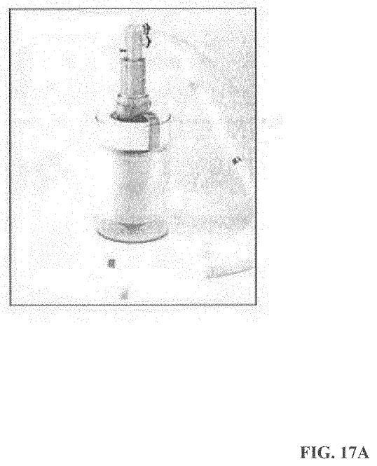

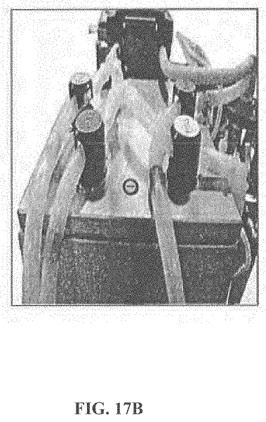

Final polymerised haemoglobin solution is filtered through a 0.5 μm depth filter (F701), a sterilizing grade 0.2 μm membrane filter (F702), and a 2nd sterilizing grade 0.2 μm membrane filter (F703), into a 20 L GE Ready Circuit flexible bag (T701). The bulk holding tank is stored under nitrogen until use. A schematic of the sterile filtration process is depicted in FIG. 16. Examples of parts used for the sterile filtration process is given in TABLE 19 below.

TABLE 19

| ID | Part | Manufacturer |
|---|---|---|
| T603 | 50 L Ready Circuit | GE Healthcare |
| T701 | 20 L Ready Circuit | GE Healthcare |
| P701 | Stainless Digital Process Pump | Masterflex |
| P602 | Stainless Digital Process Pump | Masterflex |
| F701 | 0.3 μM depth filter | Sartorius |
| F702 | 0.22 μM sterilization filter | Sartorius |
| F703 | 0.22 μM sterilization filter | Sartorius |

### Example 3: Devices and Assemblies for Manufacture and Purification Processes

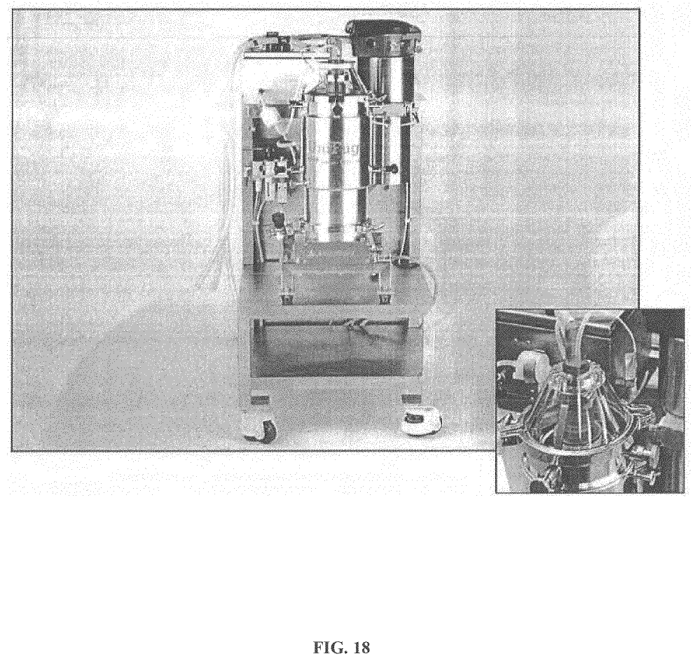

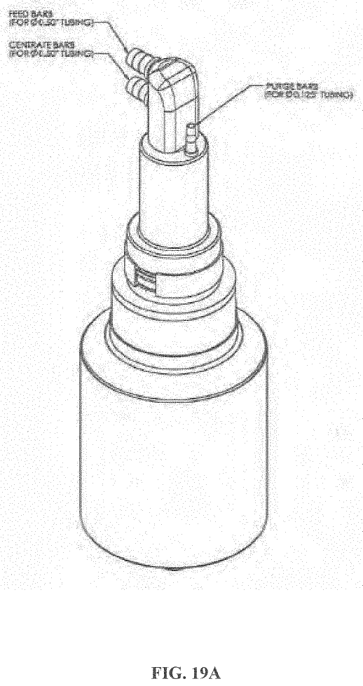

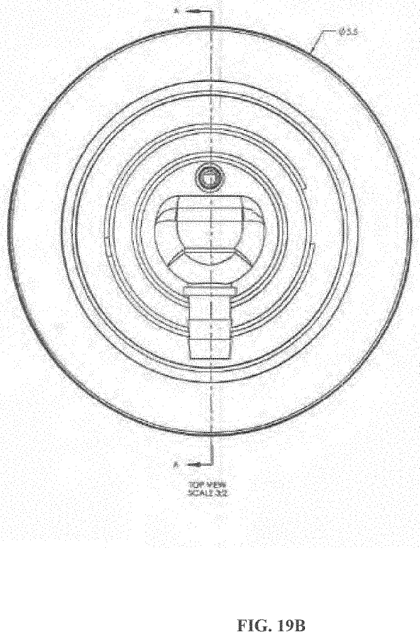

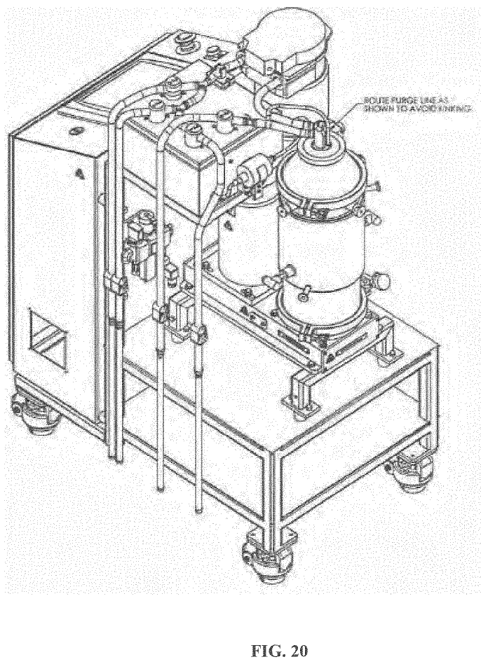

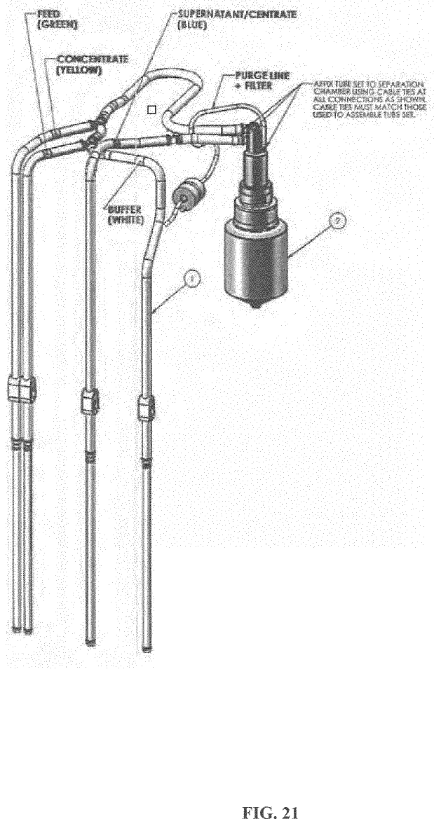

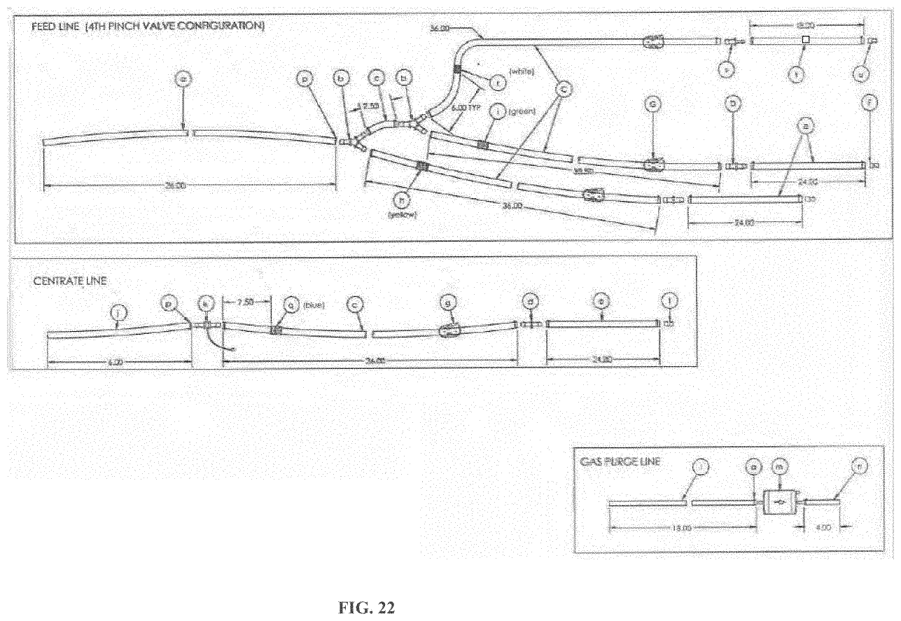

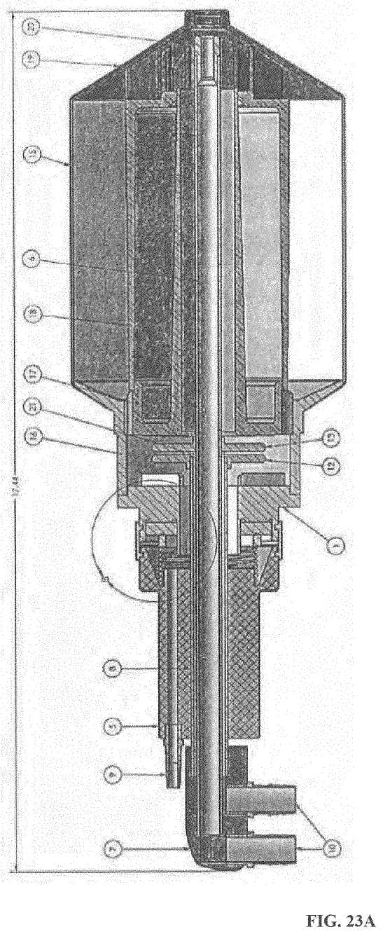

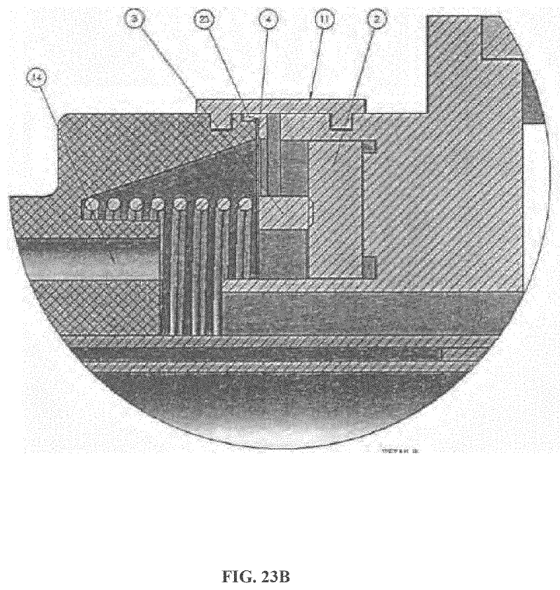

The protein (e.g. hemoglobin) purification process involves use of a separation system (FIG. 18). This separation system includes a separation chamber (FIG. 19A-FIG. 19B) and a tubeset assembly (FIG. 22) which assembles together (FIG. 21) and can be installed into a module system (FIG. 20) for extracting protein (e.g. hemoglobin) from a solution (e.g. blood). An additional device (FIG. 23A-B) can be included in the separation system for protein purification.

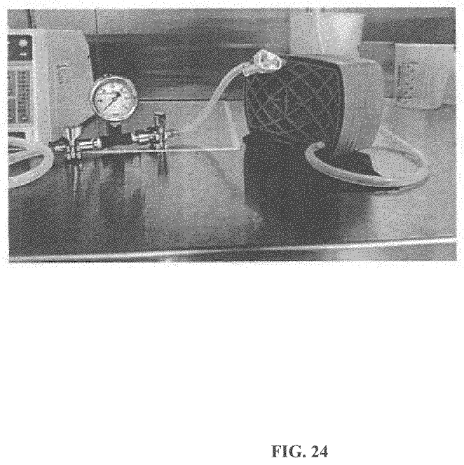

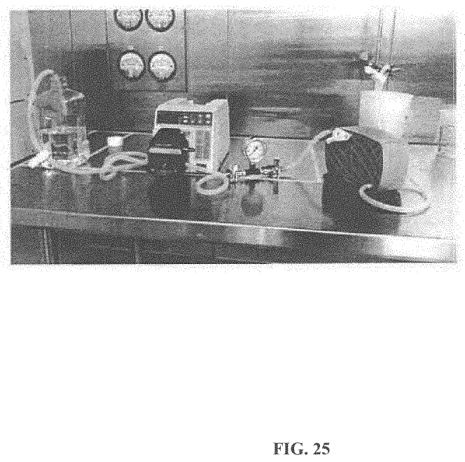

Blood depth filtration can be performed using a Millipore Clarisolve 60HX of like device (FIG. 24). The Millipore Clarisolve 60HX or like device can be connected to an assembly (FIG. 25) for blood depth filtration.

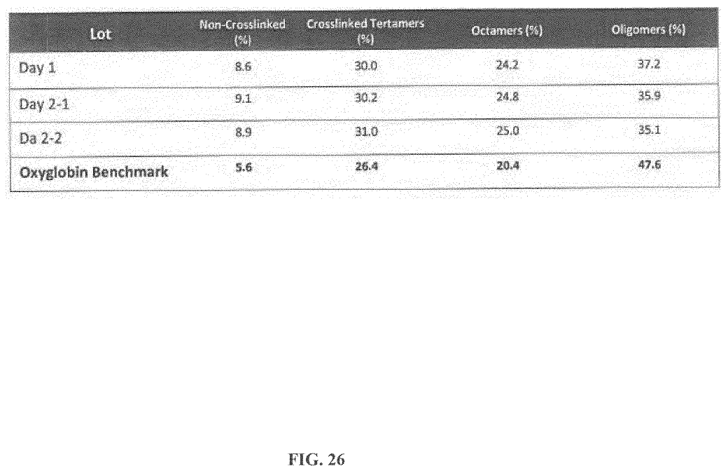

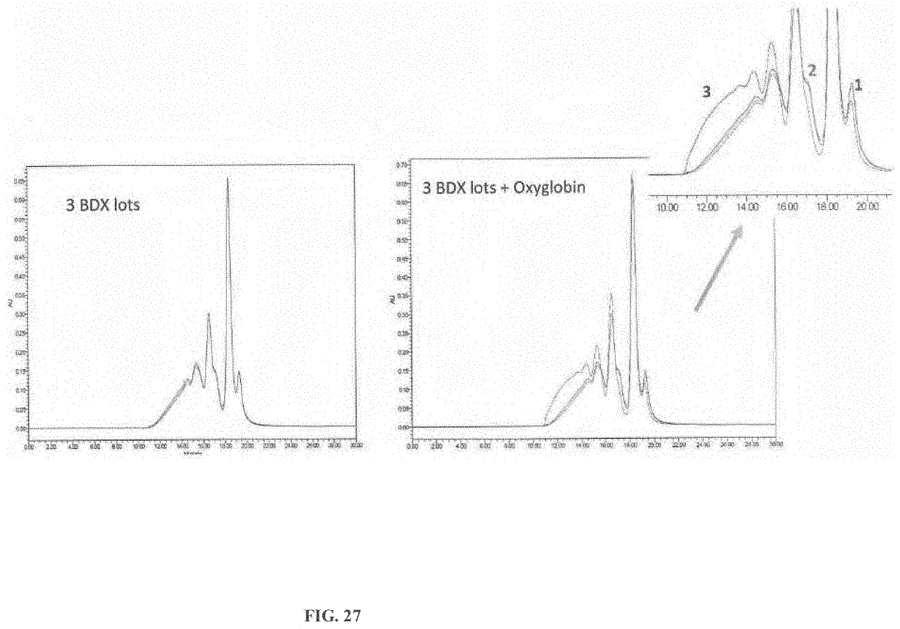

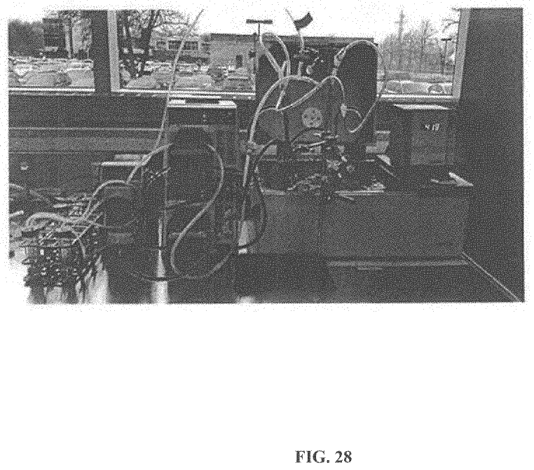

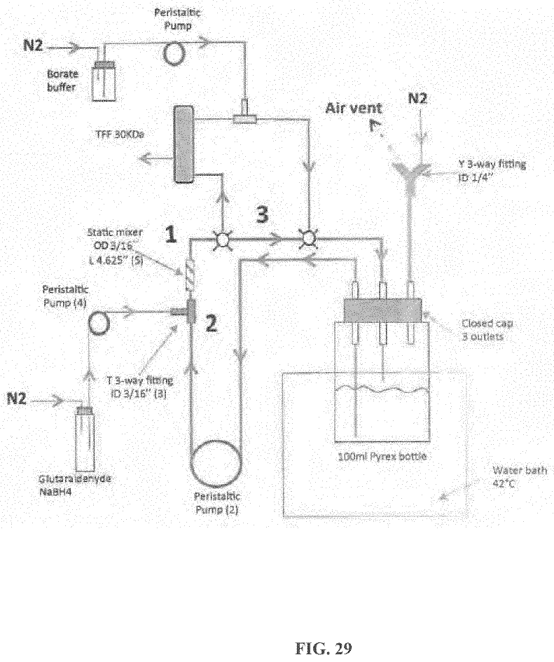

An example of a polymerization assembly is depicted as both a schematic (FIG. 29) and an image (FIG. 28). In this assembly, different glutaraldehyde/bHB proportions and types of manifold were tested. Three polymerization reactions were performed on 2 days to evaluate reproducibility with the optimized manifold. Testing parameters included 1 lot on 04 may and 2 lots on 5 May with 18 g of material per test and 29 mg gluteraldehyde per gram of hemoglobin (bHB). Testing apparatus in FIG. 28 has a static mixer 3/16″ OD×4,625 length, a T-shaped connector instead of Y-shaped to avoid Glut reflux, valves on retentate tubing for closed system conc./diaf, and continuous N2 sparging. Graphs (FIG. 27) and a chart (FIG. 26) containing protein cross-linking distribution data after polymerization processing of protein (hemoglobin) were obtained.

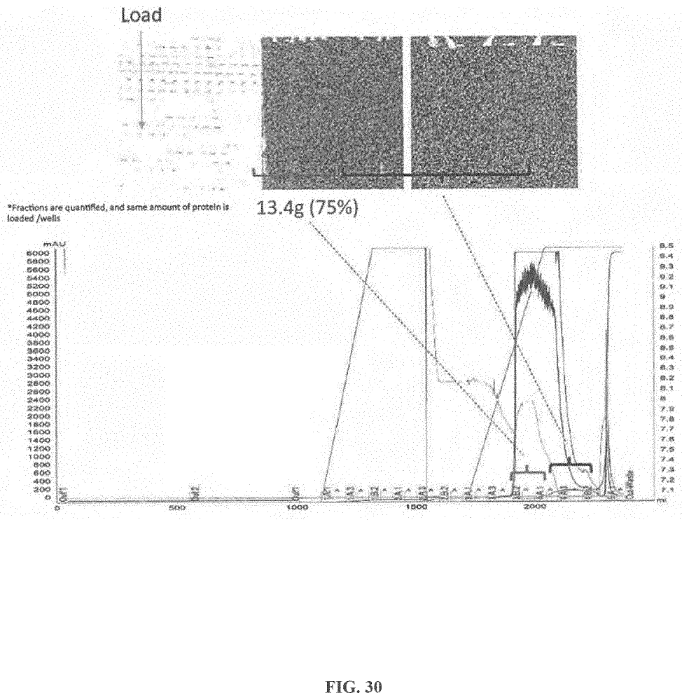

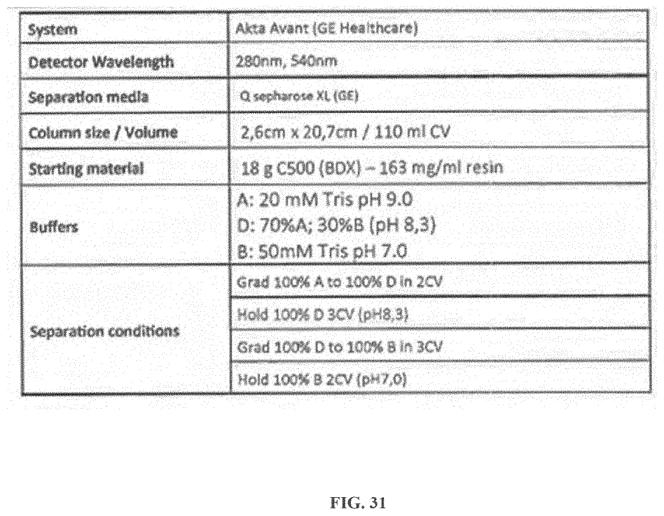

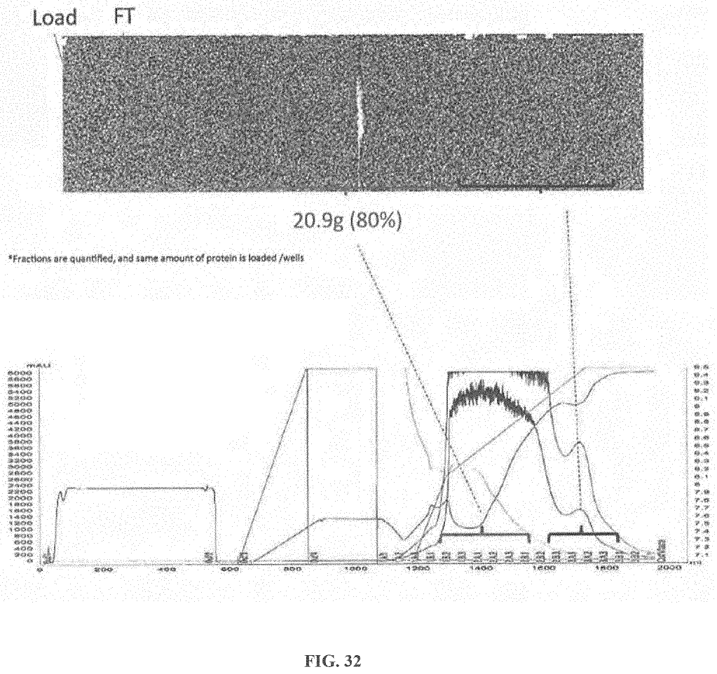

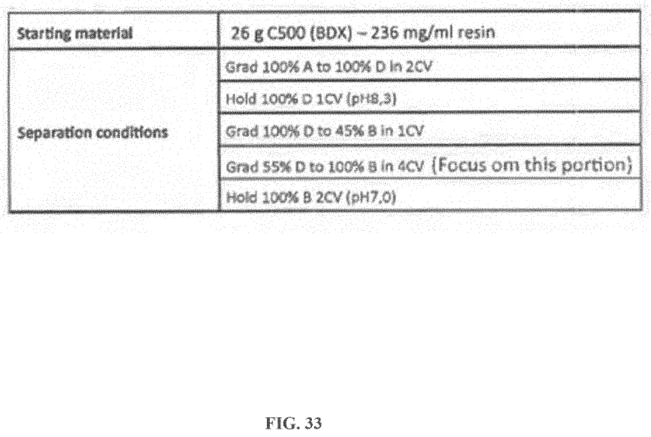

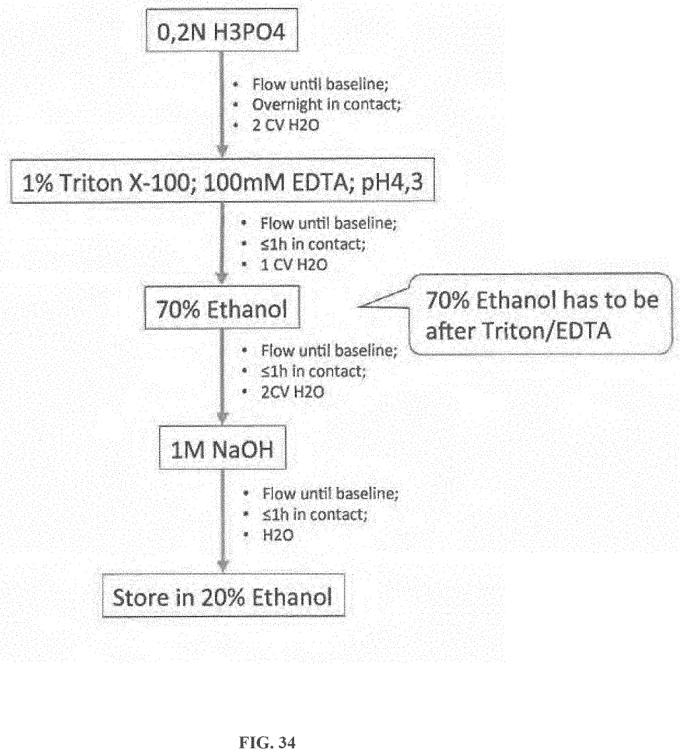

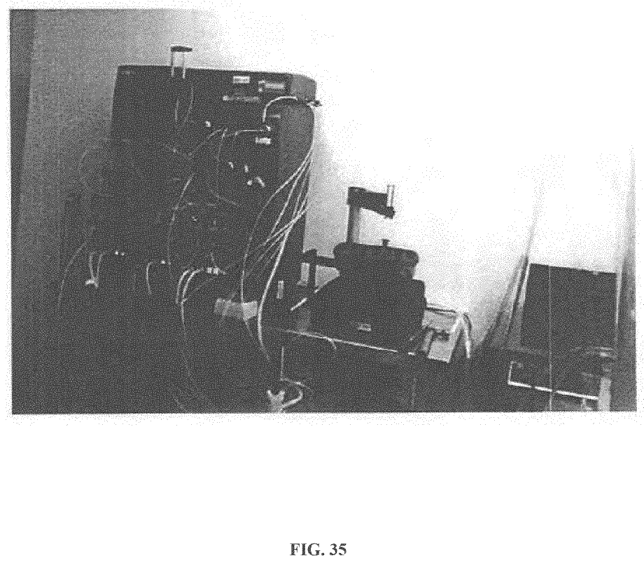

An example of a chromatography system assembly for protein purification is shown in FIG. 35. Two different gradient optimizations were performed for a C800 QEX (or equivalent) chromatography system. Graphs, images, and charts containing chromatography optimization 1 data are depicted in FIG. 30-FIG. 31. Graphs, images, and charts containing chromatography optimization 2 data are depicted in FIG. 32-FIG. 33. A flow chart for optimization of CIP of Q sepharose XL in a C800 QEX (or equivalent) chromatography system is shown in FIG. 34. In some cases of chromatography processing a 412 ml column (5 cm diameter) was loaded with 180-220 mg hemoglobin (bHB)/ml resin. Three runs were completed to process C500 1705 A. A fraction collector was used for first runs and buffers were continuously N2 sparged. The fraction collector is designed to be wrapped in an atmosbag inflated with N2. In some instances, the gradient method was optimized on a 2.6 cm diameter column.

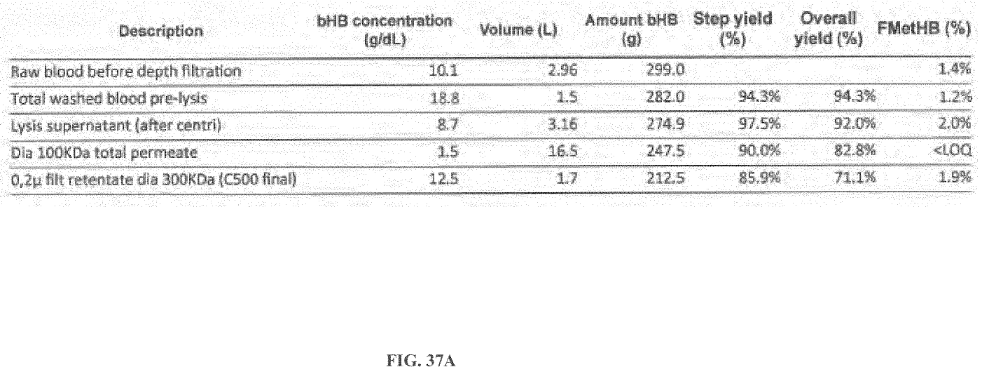

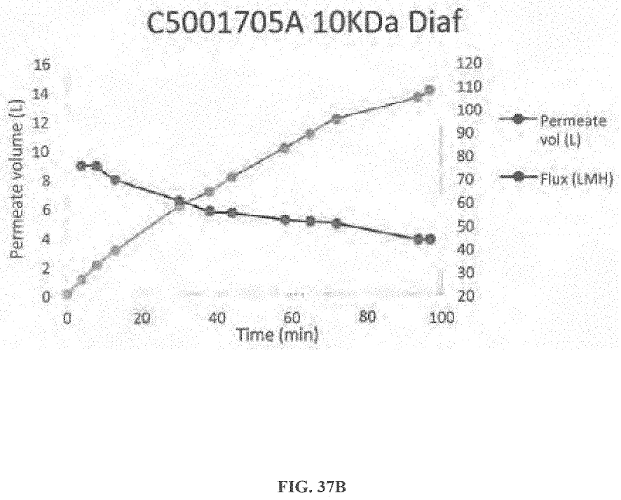

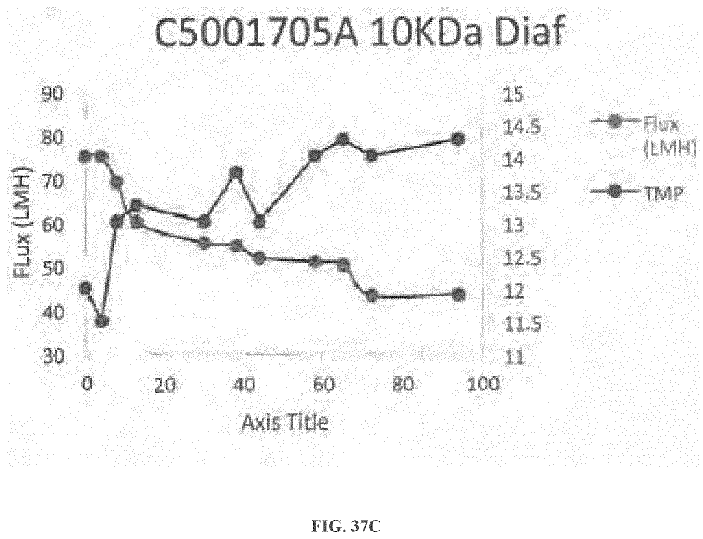

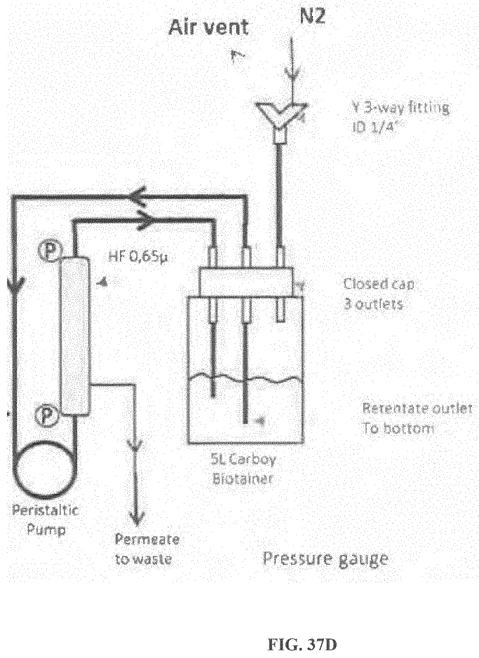

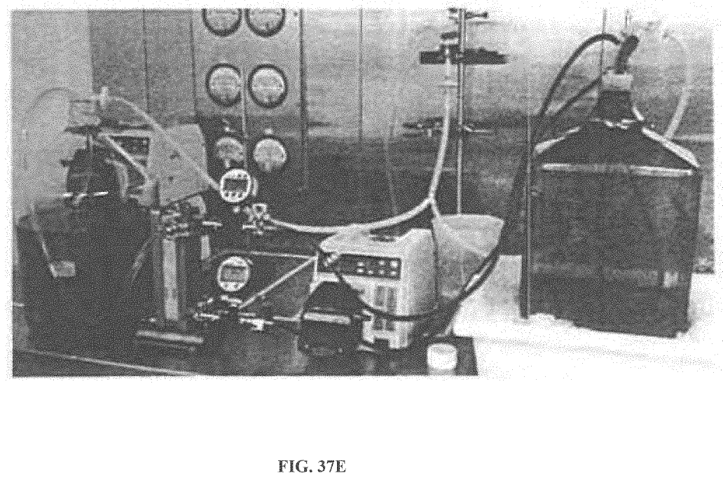

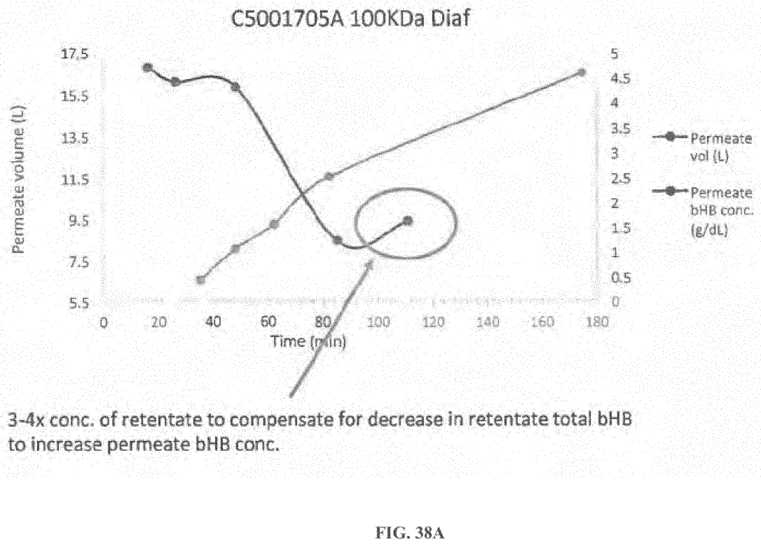

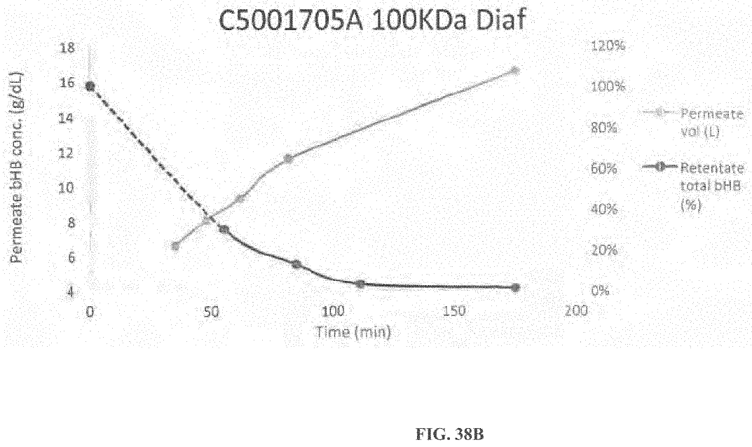

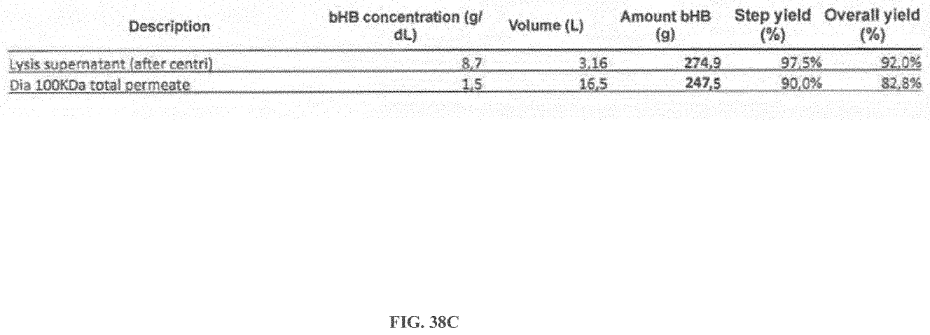

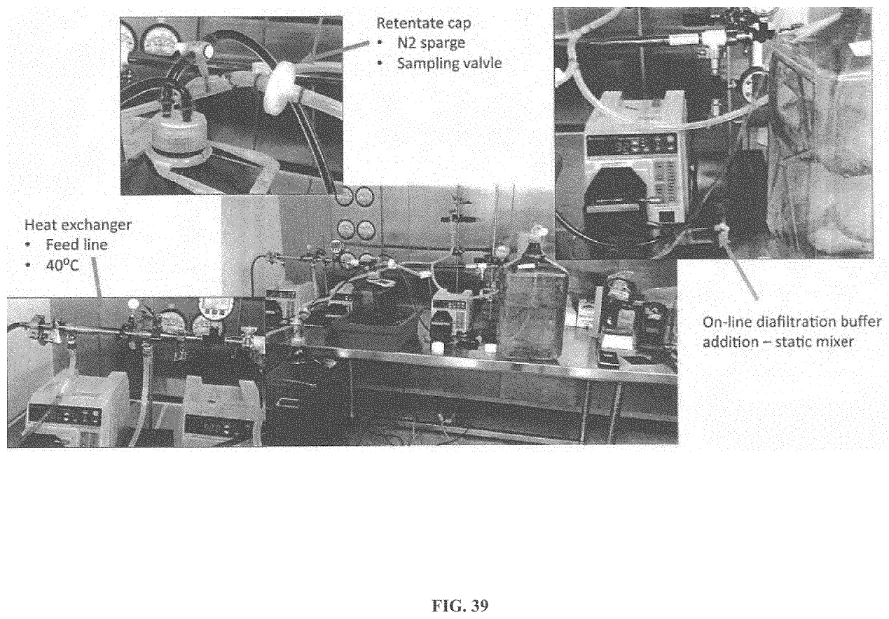

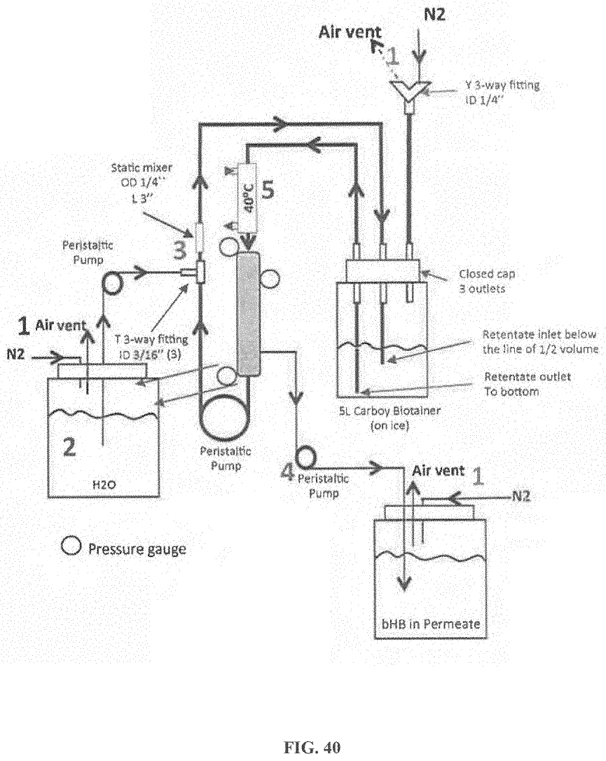

FIG. 37A-FIG. 37E depict charts, graphs, and images of a 10 KDa diafiltration process for protein purification. FIG. 38A-FIG. 38C depict a series of charts and graphs of a 100 KDa diafiltration process for protein purification. An example of an assembly for the 100 KDa diafiltration process as an image (FIG. 39) and a schematic (FIG. 40) are shown. The 100 KDa diafiltration process involves constant N2 sparging of retentate, permeate, and diafiltration buffer (H20); uses diafiltration H2O (MilliQ H₂O) at <0,005 EUml diafiltered with 10 KDa membrane; involves addition of diafiltration buffer through a T fitting with a static mixer directly in the retentate tube to improve the homogeneity of the retentate without using magnetic stirrer; includes permeate flow control with peristaltic pump to prevent formation of gel layer and flux reduction and to bridge with large pilot scale; and includes brief passage of the feed through 40° C. heat exchanger before entering the membrane which promotes increase in the proportion of the transient dimeric bUB form to improve diafiltration efficacy and yield.

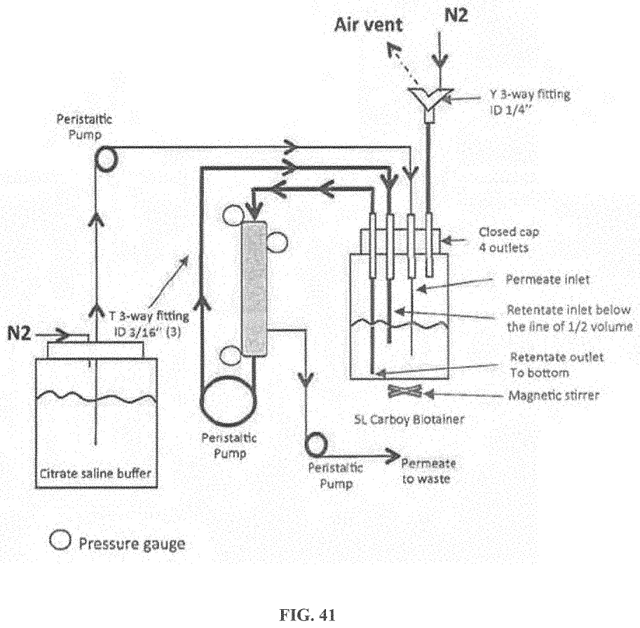

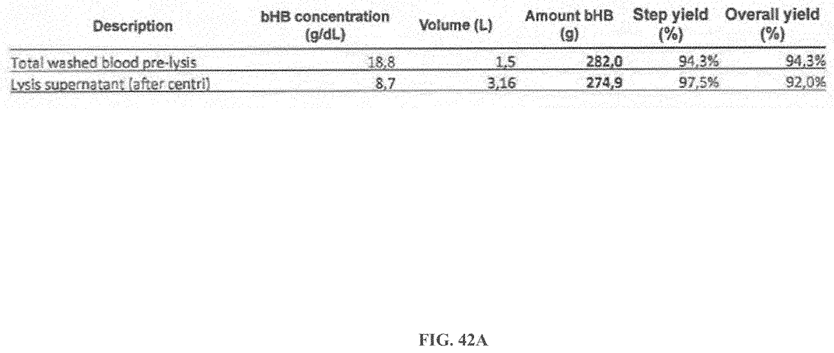

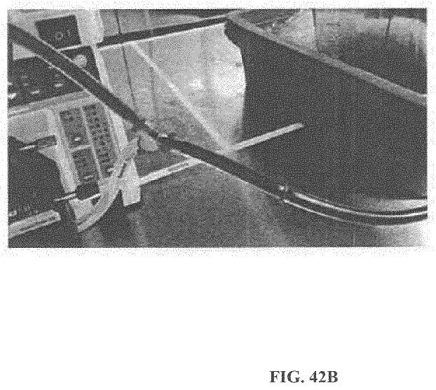

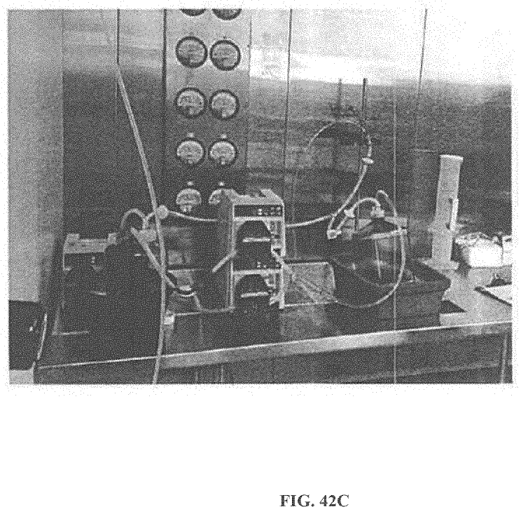

FIG. 41 depicts a schematic of a hollow fiber washing process. This process is employed on the anticoagulated blood cells before lysis. It is performed in many ways to keep the red cell intact and to ensure hemoglobin does not suffer from endotoxin and other lipid exposures. FIG. 42 A-FIG. 42C are a series of images and charts depicting data from blood washing and lysis processes.

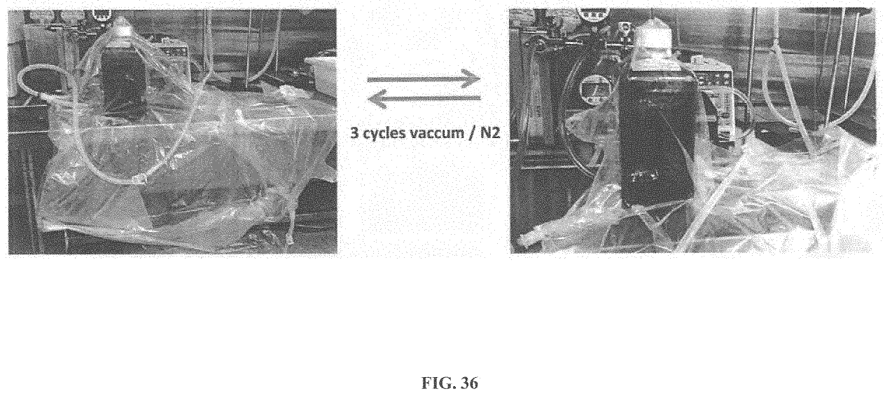

FIG. 36 is an image depicting storage of protein product C500 which can be stored at 4° C. for up to 4 weeks. This product is and intermediate material which is not chemically treated but is deoxygenated to ensure low to no oxidative activity. Sterility filtration is a benefit in the life extension to permit usable material to be drawn from the storehouse of material.

### Example 4: Modified Hemoglobin Protein Based Oxygen Carrier

Several lots of Modified Hemoglobin Protein Based Oxygen Carrier that was produced according to the disclosure were analyzed according to standard test methods. The results of lots are depicted in tables 20-23 below.

TABLE 20
Certificate Test Date: 25 Jun. 2018

| | Approved Test | | | |
|---|---|---|---|---|
| Release Tests | Methods | Unit | Specification | Test Result |
| 1. Potency | | | | |
| Total Hb | Co-oximetry | g/dL | 5.5-7.5 | 5.5 |
| MetHb | Co-oximetry | % | <10 | 2.0 |
| Oxy Hb | Co-oximetry | % | <10 | 2.0 |
| 2. Purity | | | | |
| Sterility | Sterility test | N/A | Pass | Pass |
| Endotoxin Level | Kinetic turbidimetric | EU/mL | <0.05 | <0.04 |
| Glutaraldehyde | HPLC | ug/mL | <0.15 | 0.022 |
| N-acetyl-cysteine | HPLC | % | <0.24 | Not Tested |
| Molecular Weight | | | | |
| Distribution | | | | |
| MW >500,000 | HPLC-SEC | % | <15 | Not Tested |
| MW <32,000 | HPLC-SEC | % | <5 | 3.67 |
| 3. Identity | | | | |
| Appearance | Visual | N/A | Deep Purple | Deep Purple |
| pH | Potentiometry | N/A | 7.6-7.9 @18-22° C. | 7.74 |
| Ion Concentration | | | | |
| Na⁺ | Ion selective electrode | mM | 145-160 | 160 |
| K⁺ | Ion selective electrode | mM | 3.5-5.5 | 3.9 |
| Cl⁻ | Ion selective electrode | mM | 105-120 | Not Tested |
| Ca²⁺ | Ion selective electrode | mM | 0.5-1.5 | 0.74 |

TABLE 21
Certificate Test Date: 2 Jul. 2018

| | Approved Test | | | |
|---|---|---|---|---|
| Release Tests | Methods | Unit | Specification | Test Result |
| 4. Potency | | | | |
| Total Hb | Co-oximetry | g/dL | 5.5-7.5 | 6.3 |
| Met Hb | Co-oximetry | % | <10 | 2.2 |
| Oxy Hb | Co-oximetry | % | <10 | 2.1 |
| 5. Purity | | | | |
| Sterility | Sterility test | N/A | Pass | Pass |
| Endotoxin Level | Kinetic turbidimetric | EU/mL | <0.05 | <0.04 |
| Glutaraldehyde | HPLC | ug/mL | <0.15 | 0.054 |
| N-acetyl-cysteine | HPLC | % | <0.24 | Not Tested |
| Molecular Weight | | | | |
| Distribution | | | | |
| MW >500,000 | HPLC-SEC | % | <15 | Not Tested |
| MW <32,000 | HPLC-SEC | % | <5 | 4.49 |
| 6. Identity | | | | |
| Appearance | Visual | N/A | Deep Purple | Deep Purple |
| pH | Potentiometry | N/A | 7.6-7.9 @18-22° C. | 7.71 |
| Ion Concentration | | | | |
| Na⁺ | Ion selective electrode | mM | 145-160 | 154 |
| K⁺ | Ion selective electrode | mM | 3.5-5.5 | 3.7 |
| Cl⁻ | Ion selective electrode | mM | 105-120 | Not Tested |
| Ca²⁺ | Ion selective electrode | mM | 0.5-1.5 | 0.71 |

TABLE 22
Certificate Test Date: 16 Jul. 2018

| | Approved Test | | | |
|---|---|---|---|---|
| Release Tests | Methods | Unit | Specification | Test Result |
| 7. Potency | | | | |
| Total Hb | Co-oximetry | g/dL | 5.5-7.5 | 6.7 |
| Met Hb | Co-oximetry | % | <10 | 3.1 |
| Oxy Hb | Co-oximetry | % | <10 | 3.0 |
| 8. Purity | | | | |
| Sterility | Sterility test | N/A | Pass | Pass |
| Endotoxin Level | Kinetic turbidimetric | EU/mL | <0.05 | <0.04 |
| Glutaraldehyde | HPLC | ug/mL | <0.15 | 0.044 |
| N-acetyl-cysteine | HPLC | % | <0.24 | Not Tested |
| Molecular Weight | | | | |
| Distribution | | | | |
| MW >500,000 | HPLC-SEC | % | <15 | Not Tested |
| MW <32,000 | HPLC-SEC | % | <5 | 5.95 |
| 9. Identity | | | | |
| Appearance | Visual | N/A | Deep Purple | Deep Purple |
| pH | Potentiometry | N/A | 7.6-7.9 @18-22° C. | 7.67 |
| Ion Concentration | | | | |
| Na⁺ | Ion selective electrode | mM | 145-160 | 154 |
| K⁺ | Ion selective electrode | mM | 3.5-5.5 | 3.8 |
| Cl⁻ | Ion selective electrode | mM | 105-120 | Not Tested |
| Ca²⁺ | Ion selective electrode | mM | 0.5-1.5 | 0.74 |

TABLE 23
Certificate Test Date: 23 Jul. 2018

| | Approved Test | | | |
|---|---|---|---|---|
| Release Tests | Methods | Unit | Specification | Test Result |
| 10. Potency | | | | |
| Total Hb | Co-oximetry | g/dL | 5.5-7.5 | 7.0 |
| Met Hb | Co-oximetry | % | <10 | 1.2 |
| Oxy Hb | Co-oximetry | % | <10 | 1.9 |
| 11. Purity | | | | |
| Sterility | Sterility test | N/A | Pass | Pass |
| Endotoxin Level | Kinetic turbidimetric | EU/mL | <0.05 | <0.04 |
| Glutaraldehyde | HPLC | ug/mL | <0.15 | 0.038 |
| N-acetyl-cysteine | HPLC | % | <0.24 | Not Tested |
| Molecular Weight | | | | |
| Distribution | | | | |
| MW >500,000 | HPLC-SEC | % | <15 | Not Tested |
| MW <32,000 | HPLC-SEC | % | <5 | 5.36 |
| 12. Identity | | | | |
| Appearance | Visual | N/A | Deep Purple | Deep Purple |
| pH | Potentiometry | N/A | 7.6-7.9 @18-22° C. | 7.72 |
| Ion Concentration | | | | |
| Na⁺ | Ion selective electrode | mM | 145-160 | 155 |
| K⁺ | Ion selective electrode | mM | 3.5-5.5 | 3.8 |
| Cl⁻ | Ion selective electrode | mM | 105-120 | Not Tested |
| Ca²⁺ | Ion selective electrode | mM | 0.5-1.5 | 0.71 |

### Example 5: cGMP Manufacture Modified Hemoglobin Protein Based Oxygen Carrier

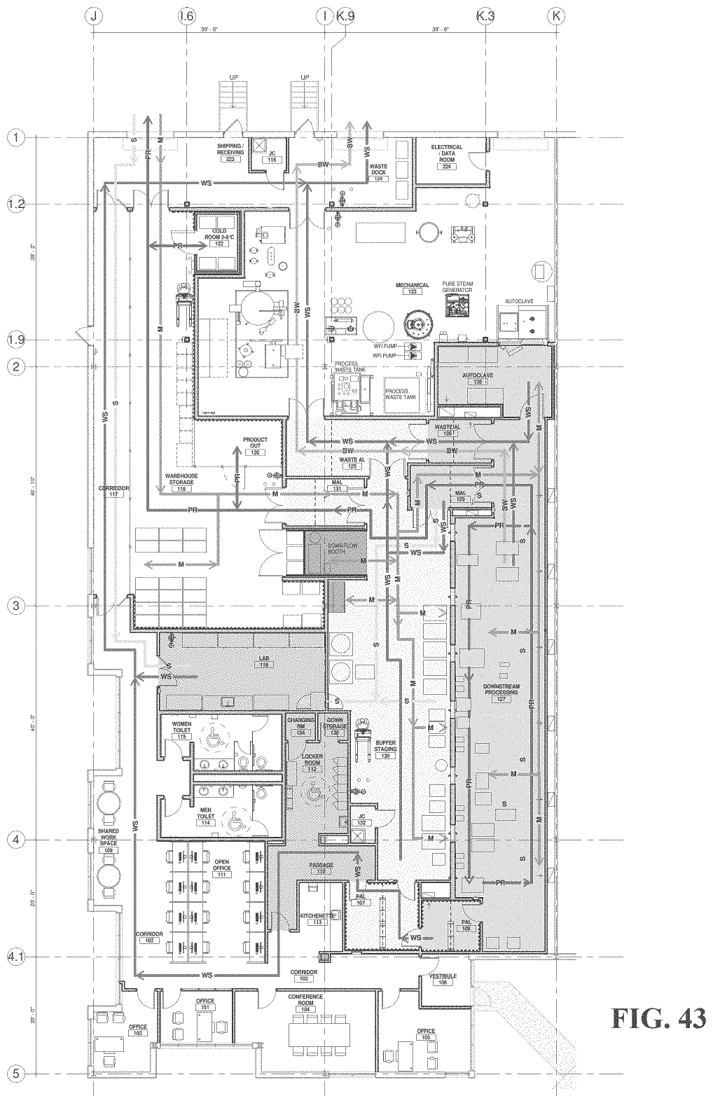

Referring to FIG. 43, a commercial scale manufacturing facility is depicted. The main manufacturing suite room 127 is designed to meet Grade CI IS08 specifications. This room is the main processing room where the hemoglobin solution(s) (i.e. raw material diluted with water) will be further purified by dedicated ion exchange chromatography according to the disclosure. The eluate is collected in an appropriate vessel so as to limit and prevent oxygen and particulate exposure. Handling and connecting are performed via tubing welders and appropriate closed containers thus mitigating all risk of room environmental exposure. Materials are them concentrated across a 30 kD TFF membrane. A bolus of NaCl buffered solution is added to the highly purified hemoglobin solution to allow for deoxygenation across a hydrophobic gas exchange membrane.

The hemoglobin solution is, filtered into the storage buffer containing an oxygen scavenger and concentrated to achieve the target hemoglobin concentration. The hemoglobin solution is then “0.2 micron filtered” into a pre-sterilized bag for storage until further processing (no open system transfers). This room also contains the process equipment for polymerizing the hemoglobin, quenching the reaction and exchanging the buffers using 30 kD membranes. Each vessel in the polymerization system also recirculates through a closed system hydrophobic gas exchange membranes to remove any oxygen introduced to the system by the addition of chemical and buffers to the process. The final polymerized hemoglobin product will be “0.22 micron filtered” into a pre-sterilized vessel. The final product will be stored in the warehouse in a secure area until release whereby it will be shipped to the contract filling facility.

In further reference to FIG. 43, the manufacturing support suite room 130 is designed to meet Grade D/IS09 specifications. This room will support the main processing area by formulating buffers used in the process. The chemicals used in the buffer formulation will be weighed in a containment hood to control particles. The buffers will be supplied to the process with tubing passed through ports in the walls and sealed with iris valves. These ports will also be used to transfer process waste fluids to a waste transfer header with will flow to a waste accumulation tank below grade.

In compliance with pharmaceutical defined SOPs, the room cleaning will be performed each working day with a quaternary ammonium “sanitant” according to the defined SOP. Monthly the rooms will be cleaned with a sporicidal agent or in response to excursions in the environmental monitoring program. The process will be performed through the use of closed pre-sterilized single-use systems. Sampling will be performed on vessels that have been tubing welded onto the system to maintain the closed system status.

As depicted in FIG. 43, the component prep room 128 is designed to meet Grade C specifications. The room will be used to prepare assemblies to use in the process of sterilization. The room includes USP purified water for rinsing materials and WFI for performing final rinse of components as needed. The room will also include an integrity tester for the pre and post-use integrity testing to be performed.

Also as shown in FIG. 43, the utility room 123 contains utilities to support the facility functions. This includes a plant steam boiler, air compressor, nitrogen/argon system, vacuum system, USP water system, pure steam generator with WFI condenser, WFI system, and the wastewater neutralization system. The mechanical side of the autoclave is also accessed from this space. The waste neutralization system will be the batch discharge type to ensure compliance with the pH discharge limits and to provide good flow for accurate measurement.

As depicted in FIG. 43, the warehouse room 1 19 is used to securely store the materials used in the production process which includes an addition secured are for final bulk product storage (room 120) and a cold room (room 122) for storage of the incoming hemoglobin solution. Incoming chemicals will be purchased with representative samples for QC testing.

The quality control lab room 1 18 will be used for the testing sample to support the ongoing operations. The bulk of the testing will be contracted out to a yet to be identified appropriate contract testing lab.

### Raw Material Source

The starting material for the process is bulk bovine hemoglobin which has been collected from a controlled donor herd. The collected red cells are washed either by diafiltration across a tangential flow filtration system or by centrifugation in a single-use disposable centrifuge. The red cells are then lysed by osmotic pressure then the hemoglobin is filtered across a 100 kD TFF membrane. The permeate is collected and concentrated across a 30 kD TFF membrane. Once the hemoglobin is at the target concentration, the hemoglobin solution is “0.22 micron filtered” into bags and stored at 2-8° C.

### Country of Origin

All animals are of US origin. The US is a GBR level II country as defined in the European Union document “Update of the Opinion of the Scientific Steering Committee on the Geographical Risk of Bovine Spongiform Encephalopathy (GBR), Adopted on 11/Jan./2002. GBR level II indicates “it is unlikely that domestic cattle in this country are infected with the BSE-agent, but it cannot be excluded.”

### Procedures for Avoiding the Risk of Cross Contamination

Whole bovine blood for processing is collected in a dedicated collection room that is separate from the remaining processing areas of the collection room or alternatively at an abattoir in controlled space. Animals from approved suppliers enter the blood collection area from the barn. All animals, from which there is any collection, will have complete documentation according to the herd management program including origin and feed status. Following bleeding or exsanguination, the animal is removed from the blood collection room for further processing back to the herd management area or in the abattoir facility.

### Isolation of Animals

Individually identified cattle arriving at the collection station or the abattoir are controlled from managed herds. In the first instance according to a standard herd management program they will be controlled as a lot before entering the dedicated blood collection area. Cattle enter through a chute which channels them directly to the collection area or a stunning platform in the case of the abattoir. The blood collection facility is separate from the primary exsanguination (if an abattoir) or collection facility at the designated facility.

### Blood Collection

Supporting documentation and identification for each animal is verified for accuracy and completeness before each collection, and the animal is inspected for any sign of disease. Blood collection is performed using a closed system. The animal (if exsanguinated) may be immobilized and if one time harvest a non-pneumatic captive bolt method maybe used for stunning. Collection at an abattoir has never used, nor will ever use, the procedure referred to as “pithing”. Immediately after stunning if at an abattoir, chain shackles are placed around a rear hoof and the animal is hoisted to a head-down position. An overhead conveyor system moves the animal carcass along the line to the collection platform. If abattoir donation, an incision in the hide is made from the angle of the jaw to the thoracic inlet; the hide is then retracted from the exposed jugular furrow by an elastic cord wrapped around the back side of the neck.

Blood is collected in a closed manner using a stainless steel trocar inserted into the jugular vein close to the vena cava. Sanitized tubing connects the sanitized trocar to a sanitized stainless steel vessel or plastic bag, which has been prepared with sodium citrate anticoagulant. Approximately 10 to 15 liters of blood is collected in a period of approximately 30-60 seconds. After the blood is collected, the trocar is removed, and the vessel is sealed. The carcass then moves out of the dedicated Oversight Collection Facility and then onto the main abattoir processing floor and cannot be returned. If at the animal management facility where animals are bleed for a controlled volume of 2 to 5 liters, animals will be restrained during donation with the blood being collected in a sterile anticoagulant charged collection bag.

Each collection vessel holds the blood of a single animal. The unique number of each collection vessel is recorded and correlated with the animal number from a unique animal ear tag. The ear tag number is further correlated with a unique abattoir animal number used to trace the cattle through the packing plant. Animals are subsequently inspected by USDA trained inspectors for evidence of disease or contamination. The inspectors are supervised by USDA trained veterinarians. If an animal is retained by the USDA staff for further examination for any reason, the blood from that animal is discarded at the abattoir. The filled collection vessels may leave the facility, and are placed in ice and loaded onto a truck for transport to the Separation Facility. If the managed donor herd, similar cataloguing is performed and bags will be collected and cooled to be transported to initial processing facilities.

### Potential for Other Tissues to Contaminate Collected Blood

The potential for contamination by other tissues is minimal because of the closed method of blood collection and through the use of well-trained operators for the controlled and documented procedure. In the abattoir the trachea and esophagus are avoided by positioning the blade of the trocar toward the blood vessel.

The site on the skull where the animal is stunned is physically distant from the location of trocar insertion (1 meter). Because of the position in which the animal is suspended during blood collection, any fluid or bone chips from the stunning site cannot come into contact with the collection site. The collected blood does not come into contact with brain, spinal cord, eye, ileum, lymph nodes, proximal colon, spleen, tonsil, dura mater, pineal gland, placenta, cerebrospinal fluid, pituitary, adrenal, distal colon, nasal mucosa, peripheral nerves, bone marrow, liver, lung or pancreas. In addition, any potential contaminating tissue would be removed during the blood pooling process at the manufacturing plant, in which the blood is sequentially filtered by an 800 μm screen, 50 μm strainer and a 60 μm depth filter. The 60 μm depth filter has a wide distribution of pore sizes; the largest pore size is 60 μm or microns.

### Water Systems

The water for injection is produced by condensing pure steam into a 2000 L storage tank maintained above 65° C. which is recirculated through a spray ball to flush all interior surfaces during operation. The hot loop does not have any direct use point but supplies a cold loop which recirculates through a heat exchanger to reduce the temperature to 25° C. One use point is at buffer preparation, and the other is in component prep to perform a final rinse before sterilization in the autoclave. The cold loop is hot water sanitized nightly for a defined time period.

The raw materials are stored at controlled room temperature except for the purified hemoglobin solution which is stored at 2 to 8° C. Standard single-use disposable product contact materials such as polypropylene, polycarbonate, silicone tubing, C-flex tubing, and bags with an inert inner layer made of ultra-low density polyethylene or equivalent are used for storage. The systems will be flushed before use to remove particulates and test for leaks before processing. If sanitation is required, the system is flushed with 0.5 M NaOH for a defined time frame then the NaOH is flushed out of the system and ensure the residual is neutralized before processing. The final product is stored at controlled room temperature.

### HVAC and Air Handling

The HVAC system provides HEPA filtered air to the clean rooms that have been cooled to reduce the moisture to less than 60% relative humidity and reheated to the desired temperature for operator comfort. The system is designed with sufficient air change rates appropriate for the classification with a pressure cascade of 0.05″ was between rooms of different classification with the main processing area at the highest pressure. The processing suite is designed with airlocks to allow the transition of people and materials to be performed with minimal impact on the processing areas. The rooms are cleaned with an approved sanitant according to a standard operating procedure. Environmental monitoring for viable and nonviable particulates will be performed on a periodic basis according to the room classification. Surface monitoring will also be performed in defined locations defined by a standard operating procedure.

### Example 6: Method for Manufacturing Concentrated, Deoxygenated Stabilized Hemoglobin Solution

### Blood Unloading, Dilution, Cell Wash, and Centrifugation

Red blood cells are washed and separated in a single use fashion according to a process similar to those displayed in FIG. 2 and the left portion of FIG. 3 or FIG. 9 and the left portion of FIG. 10, which provide exemplary schematics of systems for carrying out the blood unloading and lysing steps. The sourced blood material (defibrinated or citrated) is pumped from bags and diluted with buffer solution through a static mixer. The blood is pumped through a 50 μm blood strainer and a 60 μm depth filter to remove extraneous materials or large aggregates if needed.

The Ultrafilter skid is flushed with Buffer to waste tote prior to use. The filtered blood is further diluted then concentrated to the original loading volume then washed with 7 volumes of buffer solution using the Ultrafilter Skid.

The washed red cell solution is pumped into the centrifuge. The heavy phase containing the red blood cells (RBC) is discharged into a product collection bag tote. The cell solution is pumped from the product collection bag tote. If lysing is required, it is diluted inline with Depyrogenated Water (DPW) through a static mixer while being transferred to the RBC bag tote.

### Ultrafilter Dilution, Ultrafilter Concentration, and Diafiltration

The cell lysate is processed and purified in a single use fashion according to a process similar to those displayed in the right-hand portion of FIG. 3 and FIG. 4 or the right-hand portion of FIG. 10 and FIG. 11, which provide schematics for exemplary systems for carrying out ultrafiltration and filtration. The washed collected cell solution is sampled, tested for hemoglobin, then adjusted to 14.0-18.0 g/dL using DPW.

The 100 kDA and 30 kDA skids are flushed with DPW to waste totes prior to use. The red blood cell solution is diafiltered using a 100 kDa membrane and ˜11 volumes of DPW. This operation eliminates cellular debris larger than 100 kDa. The permeated hemoglobin-containing solution is simultaneously ultrafiltered using a 30 kDa membrane to concentrate the hemoglobin and to remove smaller debris and micro-contaminants. The hemoglobin is analyzed and ultrafiltration is continued until the intermediate is concentrated to approximately 13 g/dl. The hemoglobin, at 64 kDa, is retained (in T106) after these two steps. The concentrated hemoglobin is sampled for in-process testing.

After testing, the hemoglobin is pumped through a 0.5 μm filter and a 0.22 μm clarification filter into a receiving bag tote. The tote contents are sampled then the tote is re-located to a 2-8° C. cold room.

### Chromatography

The hemoglobin solution is chromatographically purified in a single use fashion according to a process similar to those displayed in FIG. 5 and FIG. 13, which show schematics for exemplary systems for purifying hemoglobin via chromatography, but with buffers as described below.

Pre-formulated buffers are delivered in single use bags. Single use tubing is used to supply buffers for use during the purification unit operations.

The crude hemoglobin is removed from refrigerated storage, transferred and delivered to the Purification Suite for chromatographic purification.

The column is equilibrated with Buffer A (2.42 g/L Tris, pH 9) prior to purification. The product is fed onto the column, with a bed height of 30 cm with a linear flow rate of 400 cm/hr. The column is then washed with buffer A buffer followed by a pH gradient elution with buffer A transitioning to buffer B (6.05 g/L Tris, pH 7). This buffer elutes loosely bound non-hemoglobin components which are sent to the waste stream. The product fraction is collected by recognizing a change to OD or absorbance. The column is regenerated with Buffer C (2.42 g/L Tris, 58.38 g/L NaCl pH 8.9), washed with 0.5-1.0 N NaCl and 0.5-1.0 N NaOH and stored in Ethanol:WFI, USP (20% w:v) between uses.

During the process of chromatographic purification, the eluted hemoglobin solution is diluted approximately ten-fold compared to the concentration of the crude hemoglobin solution from approximately 129 g/L to approximately 14.1 g/L. Loss of hemoglobin is low, approximately ˜10%, such that the overall yield for this step is ˜90%.

### Deoxygenation, Concentration, Diafiltration

After chromatographic purification, the hemoglobin solution is deoxygenated, concentrated and filtered in a single use fashion according to a process similar to those displayed in FIG. 6A, FIG. 6B, and FIG. 11, which provide schematics of exemplary systems for carrying out deoxygenation and filtration.

The concentrated solution is transferred to a degassing vessel and the ionic strength is adjusted to 200 mM using buffer C. The solution is then deoxygenated by diafiltration against a degassing membrane with nitrogen flowing across the opposite side of the membrane.

The deoxygenated solution is diafiltered into deoxygenated storage buffer (Phosphate solution with 2 g/L N-acetyl-L-cysteine) using a 30 kDa MWCO membrane filter and 3 diavolumes of the deoxygenated storage buffer.

The deoxygenated hemoglobin intermediate is sampled for in-process testing and filtered into a storage bag using 0.5 μm and 0.22 μm filters. This intermediate is stable for up to 60 days at 17-22° C.

Through this step of the process, the concentration of the hemoglobin solution starts at 14.1 g/L and proceeds to 125 g/L. The step yield is 98%.

### Polymerization

Following deoxygenation, the hemoglobin solution is polymerized in a single use fashion according to a process similar to those displayed in FIG. 7 or FIG. 14, which provide schematics of exemplary systems for carrying out this process. This process begins by charging deoxygenated WFI (˜1/2 intermediate volume), USP into a reactor vessel, T300, with mixing/recirculation and warmed to 42° C. The hemoglobin intermediate is added to the collection vessel T300 from T239.

The hemoglobin intermediate is chased through T239 into T300 with 2.5 volumes of additional deoxygenated WFI, USP.

Once temperature is achieved, the hemoglobin intermediate is transferred to tank T302. 0.62% Glutaraldehyde Activation Solution is added to the hemoglobin solution as it is transferred to T302 to polymerize the hemoglobin. Once the polymerization time is complete, the polymerized hemoglobin solution is cooled to 20° C.

Through this process, the concentration of the hemoglobin solution starts at 125 g/L and proceeds to 26.2 g/L. The step yield is 98%.

### Diafiltration, Concentration, Quench

Following polymerization, the hemoglobin solution is filtered, concentrated and reaction-quenched in a single use fashion according to a process similar to those displayed in FIG. 8 or FIG. 15, which provide schematics of exemplary systems for carrying out this process.

The polymerized hemoglobin solution is concentrated to ˜8 g/dL and diafiltered using a 30 kDa MWCO membrane with 3 diavolumes of Borate buffer (4.58 g/L sodium borate 10-hydrate, 0.91 g/L sodium hydroxide, pH 10.4-10.6) to adjust the pH of the solution. The polymerized hemoglobin is then recirculated across a deoxygenation filter against a cross-flow of nitrogen to remove hydrogen from the process.

The recirculating polymerized hemoglobin solution is then quenched by the addition of Quench Solution (9.00-9.95 g sodium borohydride/kg borate buffer) and slowed to recirculate through a 30 kDa MW filter and a deoxygenabon filter for 1 hour. This step concentrates the hemoglobin to approximately 70-100 g/L

The solution is buffer exchanged by diafiltration with 6 diavolumes of Diafiltration Buffer A (6.67 g/L sodium chloride, 0.30 g/L potassium chloride, 0.20 g/L calcium chloride dihydrate, 0.445 g/L sodium hydroxide. 2.02 g/L N-acetyl-L-cysteine. 3.07 g/L sodium lactate) with the continued use of a deoxygenation filter.

Finally, the material is buffer exchanged with 3 diavolumes of Diafiltration Buffer C (6. 73 g/L sodium chloride, 0.30 g/L potassium chloride, 0.20 g/L calcium chloride dihydrate, 0.512 g/L sodium hydroxide, 2.03 g/L N-acetyl-L-cysteine, 3.08 g/L sodium lactate, pH 7.75±0.15).

Through this process, the concentration of the hemoglobin solution starts at 26.2 g/L and proceeds to 85.8 g/L. The step yield is 98-99%.

### Filtration and Storage

The stabilized hemoglobin solution is filtered and stored in a single use fashion as follows.

The resulting batch of stabilized hemoglobin solution is filtered into deoxygenated Drug Substance containers using a pre-wetted (deoxygenated WFI) 0.22 μm filter and transferred to storage. The bulk stabilized hemoglobin solution is stored at 15-30° C. until later use.

Through this process, the concentration of the hemoglobin solution starts at 85.8 g/L and proceeds to 65.34 g/L. The step yield is 98-99%.

Subsequent to this step or to any other step post-deoxygenation, the stabilized hemoglobin solution is alternatively concentrated, such that the final concentration achieved is between 150-200 g/L. The methods described in this example may be used to obtain highly deoxygenated, highly concentrated, and/or substantially endotoxin-free stabilized hemoglobin solutions.

### OTHER EMBODIMENTS

While the disclosed methods and systems have been described in conjunction with the detailed description thereof, the foregoing description is intended to illustrate and not limit the scope of the disclosed systems and methods. Other aspects, advantages, and modifications are within the scope of the following claims.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. Genbank and NCBI submissions indicated by accession number cited herein are hereby incorporated by reference. All other published references, documents, manuscripts and scientific literature cited herein are hereby incorporated by reference.

While the disclosed systems and methods have been particularly shown and described with references to some embodiments thereof, it will be understood by those skilled in the art that various changes in form and details may be made therein without departing from the scope of the systems and methods encompassed by the appended claims.

1. A method for manufacturing a stabilized hemoglobin composition, comprising:
a) diluting a purified hemoglobin solution to a hemoglobin concentration of less than 30 g/L to produce a dilute hemoglobin solution;
b) deoxygenating the dilute hemoglobin solution, thereby producing a deoxygenated hemoglobin solution; and
c) polymerizing the deoxygenated hemoglobin solution, thereby producing the stabilized hemoglobin composition.

2. The method of claim 1, wherein the stabilized hemoglobin composition is substantially endotoxin-free.

3. The method of claim 1, wherein the stabilized hemoglobin composition comprises fewer than 0.05 endotoxin units (EU) per milliliter (mL) (EU/mL).

4. The method of claim 1, wherein said stabilized hemoglobin comprises less than 0.1, 0.05, 0.04, 0.03, 0.02, or 0.01 mg/mL of dissolved oxygen.

5. The method of claim 1, wherein the hemoglobin solution is derived from a crude hemoglobin solution obtained from red blood cells.

6. The method of claim 5, wherein the red blood cells are isolated or derived from a non-human animal.

7. The method of claim 6, wherein the non-human animal is a bovine.

8. The method of claim 5, wherein the red blood cells are collected using a sterile container.

9. The method of claim 8, wherein the sterile container is a single-use bag.

10. The method of claim 8, wherein the sterile container contains an anticoagulant.

11. The method of claim 10, wherein the anticoagulant is a citrate phosphate dextrose (CPD) anticoagulant.

12. The method of claim 5, wherein the red blood cells are washed.

13. The method of claim 12, wherein washing the red blood cells comprises straining, filtering, and/or washing the red blood cells with buffer solution.

14. The method of claim 5, wherein the red blood cells are lysed, thereby producing the crude hemoglobin solution.

15. The method of claim 14, wherein the lysing of the red blood cells is by a rapid decrease in osmotic pressure resulting in cell lysis.

16. The method of claim 5, wherein the crude hemoglobin solution is purified by diafiltration, ultrafiltration, clarification, and/or chromatography, thereby producing the purified hemoglobin solution.

17. The method of claim 1, wherein the deoxygenation step comprises diafiltration against a degassing membrane with nitrogen flowing across the opposite side of the membrane.

18. The method of claim 17, wherein the diafiltration against the degassing membrane continues until the dissolved oxygen level is below 0.1 mg/mL.

19. The method of claim 17, wherein the diafiltration against the degassing membrane continues until the dissolved oxygen level is below 0.02 mg/mL.

20. The method of claim 1, wherein the deoxygenated hemoglobin solution is concentrated prior to polymerization.

21. The method of claim 1, wherein the deoxygenated hemoglobin solution is further filtered prior to polymerization.

22. The method of claim 1, wherein the deoxygenated hemoglobin solution is polymerized by cross-linking with glutaraldehyde.

23. The method of claim 1, further comprising stopping the polymerizing step by adding sodium borohydride.

24. The method of claim 1, wherein the deoxygenated hemoglobin solution is diafiltered and/or concentrated during the polymerizing step.

25. The method of claim 23, wherein the stabilized hemoglobin composition is diafiltered and/or concentrated after sodium borohydride is added.

26. The method of claim 1, wherein the stabilized hemoglobin composition is concentrated to a concentration of 50-100 g/L after polymerization.

27. The method of claim 1, wherein the stabilized hemoglobin composition is concentrated to a concentration of 100-150 g/L after polymerization.

28. The method of claim 1, wherein the stabilized hemoglobin composition is concentrated to a concentration of 150-200 g/L after polymerization.

29. The method of claim 1, wherein the stabilized hemoglobin composition comprises hemoglobin isolated or derived from a non-human animal.

30. The method of claim 29, wherein the non-human animal is a bovine.

31. The method of claim 1, wherein the stabilized hemoglobin composition is stable at an ambient temperature.

32. The method of claim 1, wherein the stabilized hemoglobin composition is stable above a temperature of at least 4° C.

33. The method of claim 2, wherein endotoxins comprise one or more of a cellular lipid, a cellular lipid layer and a lipopolysaccharide.

34. The method of claim 33, wherein the one or more of a cellular lipid, a cellular lipid layer and a lipopolysaccharide is from a human cell.

35. The method of claim 33, wherein the one or more of a cellular lipid, a cellular lipid layer and a lipopolysaccharide is from a non-human vertebrate cell.

36. The method of claim 33, wherein the one or more of a cellular lipid, a cellular lipid layer and a lipopolysaccharide is isolated from a microbe.

37. The method of claim 33, wherein the one or more of a cellular lipid, a cellular lipid layer and a lipopolysaccharide is isolated from a bacterium.

38. The method of claim 1, wherein the stabilized hemoglobin composition has an average molecular weight of 200 kilodaltons (kDa).

39. The method of claim 1, wherein the stabilized hemoglobin composition is concentrated by filtration into an electrolyte solution.

40. The method of claim 39, wherein the filtration is ultrafiltration.

41. The method of claim 39, wherein the electrolyte solution minimizes formation of Methemoglobin (MetHb).

42. The method of claim 39, wherein the electrolyte solution comprises N-acetyl-L-cysteine.

43. The method of claim 1, wherein the dilute hemoglobin solution comprises a hemoglobin concentration of less than 20 g/L.

44. The method of claim 1, wherein the dilute hemoglobin solution comprises a hemoglobin concentration of 10-20 g/L.

45. The method of claim 1, wherein the stabilized hemoglobin composition comprises:
a) less than 5% MetHb, optionally less than 1% MetHb; and/or
b) less than 10% hemoglobin dimers, optionally less than 5% hemoglobin dimers.

46. The method of claim 1, wherein the stabilized hemoglobin composition comprises at least 20% tetrameric hemoglobin, optionally at least 25% tetrameric hemoglobin, and/or at least 60% greater-than-tetrameric molecular weight hemoglobin oligomers, optionally at least 70% greater-than-tetrameric molecular weight hemoglobin oligomers.

47. The method of claim 1, wherein the stabilized hemoglobin composition comprises at least one of the following: 20-35% of the total hemoglobin being in tetrameric form; 15-20% of the total hemoglobin being in octameric form; 40-55% of the total hemoglobin being in greater-than-octameric form; less than 5% of the total hemoglobin being in dimer form; or any combination thereof.

48. The method of claim 1, wherein the stabilized hemoglobin is stabilized by contacting at least one stabilizing agent selected from the group consisting of: glutaraldehyde, succindialdehyde, activated forms of polyoxyethylene and dextran, α-hydroxy aldehydes, glycolaldehyde, N-maleimido-6-aminocaproyl-(2′-nitro, 4′-sulfonic acid)-phenyl ester, m-maleimidobenzoic acid-N-hydroxysuccinimide ester, succinimidyl 4-(N-maleimidomethyl)cyclohexane-1-carboxylate, sulfosuccinimidyl 4-(N-maleimidomethyl) cyclohexane-1-carboxylate, m-maleimidobenzoyl-N-hydroxysuccinimide ester, m-maleimidobenzoyl-N-hydroxysulfosuccinimide ester, N-succinimidyl(4-iodoacetyl)aminobenzoate, sulfosuccinimidyl(4-iodoacetyl)aminobenzoate, succinimidyl 4-(p-maleimidophenyl) butyrate, sulfosuccinimidyl 4-(p-maleimidophenyl)butyrate, 1-ethyl-3-β-dimethylaminopropyl)carbodiimide hydrochloride, N,N′-phenylene dimaleimide, a bis-imidate compound, an acyl diazide compound, an aryl dihalide compound, and combinations thereof.

49. The method of claim 1, wherein the stabilized hemoglobin has a longer half-life than non-stabilized or oxygenated hemoglobin and minimizes breakdown of tetrameric hemoglobin into dimers that cause renal toxicity.

50. The method of claim 1, wherein the stabilized hemoglobin comprises at least one subunit that is synthesized in vitro.

51. The method of claim 50, wherein the at least one subunit comprises a gamma (γ) subunit.

52. The method of claim 1, wherein the stabilized hemoglobin composition is manufactured in a single use fashion.

53. The method of claim 52, wherein the single use fashion comprises using closed, pre-sterilized, single use systems; single use product contact materials; and/or single use ultra-low density polyethylene bags.

54. The method of claim 52, wherein manufacturing the stabilized hemoglobin composition in a single use fashion limits additional exposure to endotoxins and limits or eliminates the need for NaOH purging of the manufacturing systems.

55. A system for manufacturing a stabilized hemoglobin solution comprising the means to carry out a method according to claim 1.
